SEQUENCE LISTING (135 sequences)

SEQ ID NO: 1 - protein, 9 aa, synthetic construct
SLLMWITQV

SEQ ID NO: 2 - protein, 9 aa, synthetic construct
NLVPMVATV

SEQ ID NO: 3 - protein, 9 aa, synthetic construct
VLEETSVML

SEQ ID NO: 4 - protein, 9 aa, synthetic construct
GLCTLVAML

SEQ ID NO: 5 - protein, 11 aa, Homo sapiens
CAFISFNKFYF

SEQ ID NO: 6 - protein, 16 aa, Homo sapiens
CASSPQTGTGTYGYTF

SEQ ID NO: 7 - protein, 15 aa, Homo sapiens
CASSAQTGAAYGYTF

SEQ ID NO: 8 - protein, 11 aa, Homo sapiens
CARNTGNQFYF

SEQ ID NO: 9 - protein, 15 aa, Homo sapiens
CASSAQTGAAYGYTF

SEQ ID NO: 10 - protein, 19 aa, Homo sapiens
CILRDIPFDRGSTLGRLYF

SEQ ID NO: 11 - protein, 15 aa, Homo sapiens
CASSAQTGAAYGYTF

SEQ ID NO: 12 - protein, 19 aa, Homo sapiens
CILRDIPFDRGSTLGRLYF

SEQ ID NO: 13 - protein, 16 aa, Homo sapiens
CASSPQTGTGTYGYTF

SEQ ID NO: 14 - protein, 12 aa, Homo sapiens
CAVRDLSARLMF

SEQ ID NO: 15 - protein, 11 aa, Homo sapiens
CASSSVNEQYF

SEQ ID NO: 16 - protein, 12 aa, Homo sapiens
CAMWTSYGKLTF

SEQ ID NO: 17 - protein, 13 aa, Homo sapiens
CASSLEVTGELFF

SEQ ID NO: 18 - protein, 14 aa, Homo sapiens
CAERIQTGANNLFF

SEQ ID NO: 19 - protein, 15 aa, Homo sapiens
CASSLGGGGVTEAFF

SEQ ID NO: 20 - protein, 14 aa, Homo sapiens
CATTYGRMNTEAFF

SEQ ID NO: 21 - protein, 13 aa, Homo sapiens
CAVFNTGFQKLVF

SEQ ID NO: 22 - protein, 13 aa, Homo sapiens
CAVFNTGFQKLVF

SEQ ID NO: 23 - protein, 11 aa, Homo sapiens
CALGYGNQFYF

SEQ ID NO: 24 - protein, 15 aa, Homo sapiens
CASSLLATGGNGYTF

SEQ ID NO: 25 - protein, 16 aa, Homo sapiens
CASSPQTGTGTYGYTF

SEQ ID NO: 26 - protein, 11 aa, Homo sapiens
CARNTGNQFYF

SEQ ID NO: 27 - protein, 15 aa, Homo sapiens
CASSRQTGSIYGYTF

SEQ ID NO: 28 - protein, 12 aa, Homo sapiens
CAVRDISARLMF

SEQ ID NO: 29 - protein, 11 aa, Homo sapiens
CASSSVTEAFF

SEQ ID NO: 30 - protein, 17 aa, Homo sapiens
CVVIVMYSGGGADGLTF

SEQ ID NO: 31 - protein, 11 aa, Homo sapiens
CAVAPSNDMRF

SEQ ID NO: 32 - protein, 16 aa, Homo sapiens
CASSLVSGATYNEQFF

SEQ ID NO: 33 - protein, 16 aa, Homo sapiens
CAEIPNYGGSQGNLIF

SEQ ID NO: 34 - protein, 14 aa, Homo sapiens
CASSLVGGRYGYTF

SEQ ID NO: 35 - protein, 11 aa, Homo sapiens
CAANTGNQFYF

SEQ ID NO: 36 - protein, 17 aa, Homo sapiens
CASSLMFTGVPQDTQYF

SEQ ID NO: 37 - protein, 15 aa, Homo sapiens
CASSPTTGTAYGYTF

SEQ ID NO: 38 - protein, 10 aa, Homo sapiens
CAESIGKLIF

SEQ ID NO: 39 - protein, 14 aa, Homo sapiens
CSVGHGGTNEKLFF

SEQ ID NO: 40 - protein, 12 aa, Homo sapiens
CAEYSSASKIIF

SEQ ID NO: 41 - protein, 13 aa, Homo sapiens
CASSQSPGGTQFF

SEQ ID NO: 42 - protein, 15 aa, Homo sapiens
CLVGDGKSDGQKLLF

SEQ ID NO: 43 - protein, 14 aa, Homo sapiens
CSVGQGGTNEKLFF

SEQ ID NO: 44 - protein, 11 aa, Homo sapiens
CAEDNNARLMF

SEQ ID NO: 45 - protein, 13 aa, Homo sapiens
CSARDGTGNGYTF

SEQ ID NO: 46 - protein, 10 aa, Homo sapiens
CAVYGQNFVF

SEQ ID NO: 47 - protein, 13 aa, Homo sapiens
CASSQSPGGTQYF

SEQ ID NO: 48 - protein, 11 aa, Homo sapiens
CAEDSNARLMF

SEQ ID NO: 49 - protein, 15 aa, Homo sapiens
CAVRDTRDSSYKLIF

SEQ ID NO: 50 - protein, 15 aa, Homo sapiens
CAISEDITVAPEQYF

SEQ ID NO: 51 - protein, 15 aa, Homo sapiens
CAVDILTGGGNKLTF

SEQ ID NO: 52 - protein, 14 aa, Homo sapiens
CASGPYEGNQPQHF

SEQ ID NO: 53 - protein, 12 aa, Homo sapiens
CAAGGSQGNLIF

SEQ ID NO: 54 - protein, 14 aa, Homo sapiens
CASSLTGTFRGYTF

SEQ ID NO: 55 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCAFISFNKFYFGSGTKLNVKPN

SEQ ID NO: 56 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPN

SEQ ID NO: 57 - protein, 117 aa, Homo sapiens
DAKTTQPNSMESNEEEPVHLPCNHSTISGTDYIHWYRQLPSQGPEYVIHGLTSNVNNRMASLAIAEDRKSSTLILHRATLRDAAVYYCILRDIPFDRGSTLGRLYFGRGTQLTVWPD

SEQ ID NO: 58 - protein, 111 aa, Homo sapiens; identical to SEQ ID NO: 55

SEQ ID NO: 59 - protein, 111 aa, Homo sapiens; identical to SEQ ID NO: 56

SEQ ID NO: 60 - protein, 117 aa, Homo sapiens; identical to SEQ ID NO: 57

SEQ ID NO: 61 - protein, 109 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAESIGKLIFGQGTTLQVKPD

SEQ ID NO: 62 - protein, 113 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDLSARLMFGDGTQLVVKPN

SEQ ID NO: 63 - protein, 111 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCAMWTSYGKLTFGQGTILTVHPN

SEQ ID NO: 64 - protein, 111 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPN

SEQ ID NO: 65 - protein, 113 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGDGKSDGQKLLFARGTMLKVDLN

SEQ ID NO: 66 - protein, 113 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAERIQTGANNLFFGTGTRLTVIPY

SEQ ID NO: 67 - protein, 113 aa, Homo sapiens; identical to SEQ ID NO: 66

SEQ ID NO: 68 - protein, 113 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFCAVFNTGFQKLVFGTGTRLLVSPN

SEQ ID NO: 69 - protein, 113 aa, Homo sapiens; identical to SEQ ID NO: 68

SEQ ID NO: 70 - protein, 109 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAESIGKLIFGQGTELSVKPN

SEQ ID NO: 71 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCALGYGNQFYFGTGTSLTVIPN

SEQ ID NO: 72 - protein, 110 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEDNNARLMFGDGTQLVVKPN

SEQ ID NO: 73 - protein, 110 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAVYGQNFVFGPGTRLSVLPY

SEQ ID NO: 74 - protein, 111 aa, Homo sapiens; identical to SEQ ID NO: 64

SEQ ID NO: 75 - protein, 111 aa, Homo sapiens; identical to SEQ ID NO: 56

SEQ ID NO: 76 - protein, 111 aa, Homo sapiens; identical to SEQ ID NO: 56

SEQ ID NO: 77 - protein, 113 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDISARLMFGDGTQLVVKPN

SEQ ID NO: 78 - protein, 117 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVIVMYSGGGADGLTFGKGTHLIIQPY

SEQ ID NO: 79 - protein, 112 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVAPSNDMRFGAGTRLTVKPN

SEQ ID NO: 80 - protein, 111 aa, Homo sapiens; identical to SEQ ID NO: 55

SEQ ID NO: 81 - protein, 110 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCADDSNARLMFGDGTQLVVKPN

SEQ ID NO: 82 - protein, 111 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAETLSGYALNFGKGTSLLVTPH

SEQ ID NO: 83 - protein, 110 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAENRYSTLTFGKGTMLLVSPD

SEQ ID NO: 84 - protein, 110 aa, Homo sapiens
GQQLNQSPQSMFIQEGEDVSMNCTSSSIFNTWLWYKQDPGEGPVLLIALYKAGELTSNGRLTAQFGITRKDSFLNISASIPSDVGIYFCAGQPTTPLVFGKGTRLSVIAN

SEQ ID NO: 85 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNVPNDYKLSFGAGTTVTVRAN

SEQ ID NO: 86 - protein, 116 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDTRDSSYKLIFGSGTRLLVRPD

SEQ ID NO: 87 - protein, 118 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPQTGTGTYGYTFGSGTRLTVVEDL

SEQ ID NO: 88 - protein, 117 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSAQTGAAYGYTFGSGTRLTVVEDL

SEQ ID NO: 89 - protein, 117 aa, Homo sapiens; identical to SEQ ID NO: 88

SEQ ID NO: 90 - protein, 117 aa, Homo sapiens; identical to SEQ ID NO: 88

SEQ ID NO: 91 - protein, 118 aa, Homo sapiens; identical to SEQ ID NO: 87

SEQ ID NO: 92 - protein, 118 aa, Homo sapiens; identical to SEQ ID NO: 87

SEQ ID NO: 93 - protein, 118 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGHGGTNEKLFFGSGTQLSVLEDL

SEQ ID NO: 94 - protein, 114 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSVNEQYFGPGTRLTVTEDL

SEQ ID NO: 95 - protein, 116 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLEVTGELFFGEGSRLTVLEDL

SEQ ID NO: 96 - protein, 116 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQSPGGTQFFGPGTRLTVLEDL

SEQ ID NO: 97 - protein, 116 aa, Homo sapiens; identical to SEQ ID NO: 96

SEQ ID NO: 98 - protein, 117 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASSLGGGGVTEAFFGQGTRLTVVEDL

SEQ ID NO: 99 - protein, 116 aa, Homo sapiens
DADVTQTPRNRITKTGKRIMLECSQTKGHDRMYWYRQDPGLGLQLIYYSFDVKDINKGEISDGYSVSRQAQAKFSLSLESAIPNQTALYFCATTYGRMNTEAFFGQGTRLTVVEDL

SEQ ID NO: 100 - protein, 117 aa, Homo sapiens; identical to SEQ ID NO: 98

SEQ ID NO: 101 - protein, 116 aa, Homo sapiens; identical to SEQ ID NO: 99

SEQ ID NO: 102 - protein, 118 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGQGGTNEKLFFGSGTQLSVLEDL

SEQ ID NO: 103 - protein, 117 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLLATGGNGYTFGSGTRLTVVEDL

SEQ ID NO: 104 - protein, 118 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSARDGTGNGYTFGSGTRLTVVEDL

SEQ ID NO: 105 - protein, 118 aa, Homo sapiens; identical to SEQ ID NO: 104

SEQ ID NO: 106 - protein, 116 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQSPGGTQYFGPGTRLTVLEDL

SEQ ID NO: 107 - protein, 118 aa, Homo sapiens; identical to SEQ ID NO: 87

SEQ ID NO: 108 - protein, 117 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSRQTGSIYGYTFGSGTRLTVVEDL

SEQ ID NO: 109 - protein, 114 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSVTEAFFGQGTRLTVVEDL

SEQ ID NO: 110 - protein, 114 aa, Homo sapiens; identical to SEQ ID NO: 109

SEQ ID NO: 111 - protein, 118 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLVSGATYNEQFFGPGTRLTVLEDL

SEQ ID NO: 112 - protein, 117 aa, Homo sapiens; identical to SEQ ID NO: 103

SEQ ID NO: 113 - protein, 118 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSARSEIGNTIYFGEGSWLTVVEDL

SEQ ID NO: 114 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASTPDVNTEAFFGQGTRLTVVEDL

SEQ ID NO: 115 - protein, 115 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVAGGDEQFFGPGTRLTVLEDL

SEQ ID NO: 116 - protein, 114 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSIQGEAFFGQGTRLTVVEDL

SEQ ID NO: 117 - protein, 119 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLAHRAGGDEQYFGPGTRLTVTEDL

SEQ ID NO: 118 - protein, 117 aa, Homo sapiens
DAGITQSPRHKVTETGTPVTLRCHQTENHRYMYWYRQDPGHGLRLIHYSYGVKDTDKGEVSDGYSVSRSKTEDFLLTLESATSSQTSVYFCAISEDITVAPEQYFGPGTRLTVTEDL

SEQ ID NO: 119 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVDLNDYKLSFGAGTTVTVRAN

SEQ ID NO: 120 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVTSGGSYIPTFGRGTSLIVHPY

SEQ ID NO: 121 - protein, 117 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDTPSGSARQLTFGSGTQLTVLPD

SEQ ID NO: 122 - protein, 115 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSSHGSTGELFFGEGSRLTVLED

SEQ ID NO: 123 - protein, 118 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSDPFRSGTDSPLHFGNGTRLTVTED

SEQ ID NO: 124 - protein, 120 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLTSSGGGPYNEQFFGPGTRLTVLED

SEQ ID NO: 125 - protein, 9 aa, Homo sapiens
ILLWQPIPV

SEQ ID NO: 126 - protein, 11 aa, Homo sapiens
LLLARAASLSL

SEQ ID NO: 127 - protein, 9 aa, Homo sapiens
TLMSAMTNL

SEQ ID NO: 128 - protein, 12 aa, Homo sapiens
LLFFWLDRSVLA

SEQ ID NO: 129 - protein, 9 aa, Homo sapiens
MQDEGQESL

SEQ ID NO: 130 - protein, 9 aa, Homo sapiens
SLDGTTTKA

SEQ ID NO: 131 - protein, 11 aa, Homo sapiens
SGMFDYDFEYV

SEQ ID NO: 132 - protein, 10 aa, Homo sapiens
HLFDSVFRFL

SEQ ID NO: 133 - protein, 9 aa, Homo sapiens
STMYYLWML

SEQ ID NO: 134 - protein, 9 aa, Homo sapiens
SLHSLKNRV

SEQ ID NO: 135 - protein, 10 aa, Homo sapiens
GMFDYDFEYV

(19) United States
(12) Patent Application Publication
(10) Pub. No.: US 2026/0027153 A1
(43) Pub. Date: Jan. 29, 2026

(54) THERAPEUTIC POLYNUCLEOTIDES ENCODING T CELL RECEPTOR (TCR) ALPHA CHAIN POLYPEPTIDES AND/OR TCR BETA CHAIN POLYPEPTIDES

(71) Applicant: The Regents of the University of California, Oakland, CA (US)

(72) Inventors: Dino Di Carlo, Los Angeles, CA (US); Owen N. Witte, Sherman Oaks, CA (US); Doyeon Koo, Los Angeles, CA (US); Zhiyuan Mao, Los Angeles, CA (US)

(73) Assignee: The Regents of the University of California, Oakland, CA (US)

(21) Appl. No.: 19/144,090

(22) PCT Filed: Jan. 16, 2024
(86) PCT No.: PCT/US2024/011697

§ 371 Date: Jun. 27, 2025

Related U.S. Application Data
(60) Provisional application No. 63/480,171, filed on Jan. 17, 2023.
(60) Provisional application No. 63/592,093, filed on Oct. 20, 2023.

(51) Int. Cl. A61K 35/17; A61K 40/11; A61K 40/32; A61K 40/42; A61K 40/46; C07K 14/725; C07K 16/08; C07K 16/30; C12N 15/85

(52) U.S. Cl. CPC A61K 35/17; A61K 40/11; A61K 40/32; A61K 40/4242; A61K 40/46; C07K 14/7051; C07K 16/088; C07K 16/30; C12N 15/85; C07K 2317/34; C07K 2317/52

(57) ABSTRACT

The invention provides methods and materials for the preparation of immune cells, including T cells engineered to express exogenous T cell receptors that target polypeptides associated with human leukocyte antigens. Embodiments of the invention include polynucleotides encoding T cell receptors that target human MEAF6 and SCAMP3 polypeptides, and engineered T cells transduced with these polynucleotides. Embodiments of the invention include polynucleotides encoding T cell receptors that target cytomegalovirus and Epstein Barr virus polypeptides, and engineered T cells transduced with these polynucleotides. Embodiments of the invention also include methods of making and using such polynucleotides and engineered T cells.

### RELATED APPLICATIONS

This application claims priority to U.S. Provisional Patent Application No. 63/480,171 filed on Jan. 17, 2023 and U.S. Provisional Patent Application No. 63/592,093 filed on Oct. 20, 2023 which are hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with government support under CA256084 and CA092131 awarded by the National Institutes of Health. The government has certain rights in the invention

### TECHNICAL FIELD

The technical field generally relates to methods of screening for T cell receptor (TCR) repertoires using shaped particles or nanovials using flow cytometers or fluorescence activated cell sorters (FACS). The technical field also relates to the sequencing of TCRs of sorted cells from the shaped particles or nanovials. An expanded repertoire of functional TCRs that target viral antigens or cancer-specific antigens (e.g., prostate cancer) were discovered using the platform.

### REFERENCE TO AN ELECTRONIC SEQUENCE LISTING

The contents of the electronic sequence listing (2024-042-2_SeqListing.xml; Size: 122 kilobytes; and Date of Creation: Jan. 16, 2024) is herein incorporated by reference in its entirety.

### BACKGROUND

Engineered cell therapies are increasingly the focus of research and development activities. It is believed that engineered cell therapies will become a pillar of medicine along with molecular and genetic interventions. There have been encouraging successes in the use of engineered T cell-based therapies, including T cell receptor (TCR) immunotherapy in treating cancer. These approaches use endogenous signaling activity in T cells and rely on the recognition of cancer-associated antigens that are presented as peptides associated with major histocompatibility complex (MHC) on the surface of tumor cells. Engineered TCRs have demonstrated efficacy in treating multiple types of tumors including melanoma, sarcoma, and leukemia.

One technical hurdle for developing effective TCR immunotherapy is to identify reactive TCRs that can recognize targets with sufficient affinity and potency. T cells have one of the most diverse sequence repertoires (10⁸-10²⁰) to respond to a wide variety of pathogens. Current tools for enriching and screening cognate T cell populations rely mostly on TCR affinity or function, as defined by surface or intracellular markers of lymphocyte activation. Peptide-MHC (pMHC) multimer (e.g., tetramer) staining is the conventional method to specifically label T cells with cognate TCRs benefiting from the avidity effect when four pMHC monomers are linked through a tetrameric streptavidin backbone. However, pMHC multimer staining does not take into account the functional stages of the T cells, and TCR affinity is not always correlated with activation or cytotoxicity.

An alternative way to isolate reactive T cells is through both extracellular and intracellular activation markers. The isolation of T cells following stimulation with antigen-presenting cells based on activation biomarkers can be achieved without the knowledge of specific epitopes and the readouts of these markers are based largely on functional activation of the T cell. Despite improvement in these activation-based selection techniques and better choices of markers, some of the T cells isolated by surface markers have been reported to be “bystander T cells”, meaning they were not able to respond to antigens in a reconstructed experiment. Techniques based on intracellular markers, on the other hand, require cell fixation and permeabilization leading to less RNA recovery at lower quality and still suffer from high false positivity, as non-cytotoxic cells can secrete interferons (IFNs) and tumor necrosis factors (TNFs). Secreted granzyme B is thought to be a specific marker for epitope-induced cytotoxic cells, but traditional methods like FACS and ELISPOT are not compatible with sorting of live single cells. An ideal technology would combine antigen-specific enrichment and secretion-based screening to achieve both highly specific identification of functional TCR sequences along with the knowledge of their cognate target epitopes.

### SUMMARY

According to one embodiment, a particle-based system is used to confine living cells and specifically T cells in small nanoliter-volume cavities located within hydrogel microparticles (also referred to “nanovials” herein) and capture secreted molecules on the nanovial surface within the cavity. The nanovial technology was adapted to achieve combined antigen-specific capture and functional activation-based high-throughput analysis and sorting of live single T cells based on secreted molecules. Each nanovial acts as both an artificial antigen-presenting cell that presents pMHC molecules at high valency within the cavity to capture with high avidity and activate cells with cognate TCRs, and as a capture site for secreted molecules, allowing accurate measurement of secreted effector molecules, such as cytokines or granzyme B. The secreted molecules that are captured in the nanovial may be labeled with fluorescent detection antibodies while captured cells may be labeled with a viability dye and fluorescent antibodies targeting additional cytotoxic T cell-specific surface markers (e.g., CD3, CD8).

To recover TCR sequences in an epitope-specific manner, live cells located on the nanovials (and within the cavity) may be sorted based on CD3 and CD8 expression and secretion (i.e., IFN-γ, granzyme B), followed by single-cell sequencing to construct a single-cell TCR library with matching αβ-chains. Corresponding antigen-specific information and secretion amount are linked to each TCR sequence using oligonucleotide feature barcodes encoding the specific pMHC molecules on the nanovial and, in some embodiments, an antibody targeting the secretion detection antibody. The secretion detection antibody can also be directly labeled with the oligonucleotide feature barcode. This platform enabled the discovery of an expanded number of viral-epitope-specific cognate TCRs compared to tetramers. Moreover, the platform enabled the identification and recovery of rare prostate cancer-specific functional TCRs that emerged as promising candidates when linking single-cell secretion to each TCR sequence. The system and platform also enables ranking TCRs based on expected function based on the amount of oligonucleotide feature barcodes associated with the secretion detection antibody. The system and platform has the ability to analyze and sort thousands of rare antigen-reactive T cells and construct a TCR library with significantly expanded breadth, TCR functional annotation, and epitope-specific annotation compared to a library constructed from cells enriched using current affinity or activation-based approaches that were tested in parallel.

In one embodiment, nanovials coated with peptide MHC molecules are used to isolate T cells based on TCR binding and measure their secretion of secreted molecules (e.g., cytokines or granzyme B). Secreting T cells located on/in the nanovials can be enriched by flow cytometry and sequenced downstream using single-cell sequencing approaches, such as microfluidic droplet-based single-cell RNA-seq. The TCR repertoire can be linked to the peptide MHC molecules on the nanovials by barcoding the nanovials with oligonucleotides that correspond to the specific peptide MHC. The MHC-nanovial platform, coupled with oligo-tagged antibodies, allows the encoding and quantification of secreted molecules, such as cytokines or granzyme B on the single-cell level and linking this valuable secretion quantification data with the TCR sequence information. In one particular application, a new set of TCRs specific to cytomegalovirus (CMV). Epstein-Barr virus (EBV), and prostate cancer-specific antigens was identified with combined measurement of affinity and function to elicit cytokine secretion, which has therapeutic potential. Using secreted granzyme B as a specific marker for cytotoxic T cells, a set of TCRs targeting prostate cancer-enhanced splicing-derived epitopes were also recovered and verified by cytokine release assays upon exposure to antigen-presenting cells. Specifically, epitopes derived from MEAF6 splicing variants were previously reported to be enriched in both small cell lung cancer (SCLC) and neuroendocrine prostate cancer (NEPC). Those epitopes are also predicted to have high binding affinities to all common HLA-A alleles. The TCR isolated against the MEAF6 splicing variant can be introduced into cells to serve as a therapeutic targeting multiple cancer types and HLA-alleles all at once.

In one embodiment, a shaped particle system includes a plurality of three-dimensional shaped particles, each three-dimensional shaped particle having a cavity formed therein that comprises an opening to an external environment of the three-dimensional shaped particle. Each three-dimensional shaped particle includes the following: (1) a plurality of peptide-major histocompatibility complex (pMHC) monomers of a particular type disposed on the three-dimensional shaped particle in the cavity. (2) secretion capture antibodies disposed on the three-dimensional shaped particle in the cavity, and (3) an oligonucleotide barcode that is specific to the particular type of pMHC disposed on the three-dimensional shaped particle in the cavity.

In another embodiment, a method of using the shaped particle system for TCR analysis includes loading T cells into the plurality of three-dimensional shaped particles and allowing the T cells to bind to the pMHC monomers. The T cells then produce secretions that are captured with the secretion capture antibodies. The three-dimensional shaped particles loaded with secreting T cells are exposed to one or more fluorescent reporter(s) or primary antibodies specific to the secretions bound by the secretion capture antibodies. Additional fluorescent reporter(s) or antibodies specific to TCRs or cell surface markers (or dyes that are indicative of cell viability) may also be exposed to the plurality of three-dimensional shaped particles. The three-dimensional shaped particles are then sorted in a fluorescence activated cell sorter (FACS) based on the presence or abundance of the fluorescent reporter(s) or fluorescent antibodies in the three-dimensional shaped particles. The sorting of three-dimensional shaped particles/T cells may be performed by gating on a threshold of multiple fluorescent signal(s) of the fluorescent reporter(s) or antibodies specific to one or more of the secretions, a T cell receptor or a T cell surface marker. In a related embodiment, the fluorescent reporter(s) or primary antibodies may comprise an oligo-nucleotide barcode in addition or instead of the fluorescent reporter(s).

In another embodiment, the plurality of three-dimensional shaped particles loaded with secreting T cells are exposed to a barcoded secondary antibody that is specific to the one or more fluorescent reporter(s) or fluorescent antibodies specific to the secretions. The barcoded secondary antibody may include an oligo-nucleotide barcoded antibody. The oligo-nucleotide barcoded antibody allows for the quantification of the secretions secreted by the respective T cells contained in the three-dimensional shaped particles.

In another embodiment, single-cell RNA sequencing is performed on the T cells contained within the three-dimensional shaped particles to generate a single-cell matched alpha and beta chain TCR sequence associated for each T cell within the three-dimensional shaped particles. The respective TCR sequences are associated with particular T cells using the oligonucleotide barcode that is specific to the particular type of pMHC disposed on the three-dimensional shaped particle in the cavity.

In another embodiment, an analysis of the secretion levels of the T cells within individual three-dimensional shaped particles is performed based on RNA sequencing of the barcoded primary or secondary antibody (e.g., oligo-nucleotide barcoded antibody). The matched alpha and beta chain TCR sequences may then be linked to a secretion level in respective T cells. For example, the TCR sequences may be ranked for potential therapeutic use based on secretion levels.

### BRIEF DESCRIPTION OF THE DRAWINGS

FIG. 1A schematically illustrates a single three-dimensional particle having a cavity that opens to the external environment via an opening (e.g., a single opening in one preferred embodiment). The inner surface of the cavity includes an oligonucleotide barcode, pMHC molecules, and secretion capture antibodies.

FIG. 1B is a magnified view of the inner surface of the cavity of a three-dimensional particle that is used for performing single antigen-specific T cell secretion assays. A T cell is shown bound to the pMHC molecules and releases secretions that are captured by secretion capture antibodies located on the inner surface of the cavity of the three-dimensional particle. Fluorescent reporters or detection antibodies may be used to detect or label secretions. T cell receptors or T cell surface markers. A barcoded secondary antibody may also be used to quantify the amount of secretion secreted by the T cells.

FIGS. 2A-2H illustrates an overview of high-throughput analysis and isolation of antigen-specific T cells followed by recovery of a single-cell TCR library. FIG. 2A illustrates the optional pre-expansion of antigen-reactive T cells by exposure of PBMCs with target peptides for 7 days. FIG. 2B shows the functionalization of nanovials with secretion capture antibodies, pMHC monomers and oligonucleotide barcodes via streptavidin-biotin chemistry.

FIG. 2C shows the loading of cognate T cells into the cavities of nanovials in a well plate and removal of unbound cells using a cell strainer. FIG. 2D shows the activation of T cells for 3 hours and secretion capture in the cavity of nanovials. FIG. 2E illustrates labeling of captured cytokines and cell surface markers with fluorescent detection antibodies, followed by oligonucleotide barcoded antibodies against secreted markers. FIG. 2F illustrates sorting of T cells on nanovials based on viability. CD3/CD8 expression and secretion signal. FIG. 2G shows the compartmentalization of sorted population into droplets with a cell barcode bead in the 10× Chromium system for the construction of matched V(D)J and feature barcode libraries (nanovial-epitope and secretion barcodes). FIG. 2H illustrates the annotation of TCR clonotypes with corresponding secretion levels and epitopes by matching feature barcodes with the conserved 10× cell barcode for single cells. Scale bars represent 50 μm.

FIGS. 3A-3D schematically illustrate the detection of antigen-specific T cells on HLA-A*02:01 restricted NY-ESO-1 pMHC labeled nanovials. FIG. 3A shows PBMCs transduced with 1G4 TCRs are captured onto NY-ESO-1 pMHC labeled nanovials with ˜94% of bound cells staining positive for anti-NGFR (NGFR is part of the engineered 1G4 TCR gene construct). Scale bar represents 50 μm. FIG. 3B illustrates the fraction of nanovials containing live cells plotted as a function of pMHC concentration for 1G4-transduced (dark dots) or untransduced PBMCs (light dots). FIG. 3C illustrates flow cytometry plots of IFN-γ secretion induced by nanovials at 3 hours for 1G4-transduced PBMCs loaded onto anti-CD45 labeled nanovials (dark dots) or NY-ESO-1 pMHC labeled nanovials (light dots). Secreting cells on pMHC-labeled nanovials sorted from the gated area are shown. Scale bar represents 50 μm. FIG. 3D illustrates the purity of recovered cognate T cells with various affinities to HLA-A*02:01 restricted NY-ESO-1 pMHC. Measurements are based on binding to nanovials (circles), nanovials with IFN-γ secretion (squares) or labeling with dual-color tetramers (diamonds) as a function of TCR.

FIGS. 4A-4H illustrate the sorting of antigen-specific cells and recovery of single-cell TCR clonotypes using nanovial, tetramer, or CD137 approaches. FIG. 4A illustrates the TCR discovery workflow and matching epitope deconvolution using nanovials along with comparison techniques. For nanovials, each TCR was matched with a corresponding oligonucleotide barcode sequence reflecting pMHC information. FIG. 4B shows a summary of single-cell TCR αβ sequencing results. Sorted cells refers to the number of cells gated and sorted as positive (see FIGS. 12A-12B for detailed gates). FIG. 4C illustrates a representative Venn diagram of recovered αβ-paired TCR sequences from three approaches with a frequency≥5 (left) or ≥2 (right). FIG. 4D shows the percent of GFP/CD8/murineTCRβ⁺ cells from NFAT-GFP reporter Jurkat cells transduced with respective TCRs and exposed to antigen-presenting cells (APCs) with exogenously added peptides. CMV1-reactive TCRs recovered by both nanovial and tetramer approaches and only nanovials or EBV-reactive TCRs recovered by nanovials are plotted. Connecting lines and an asterisk represent TCRs recovered from the same cell clonotype. Venn diagram showing 4 overlapping and 6 additional TCRs recovered by nanovials compared to CMV1 pMHC tetramers. FIG. 4E illustrates IFN-γ secretion measured by ELISA plotted following exposure of PBMCs transduced with the same 19 reactive TCRs to APCs with added peptide. No secretion was observed from the negative control group. PBMCs transduced with the same vector but without TCRs. FIG. 4F shows the workflow representing direct ex vivo analysis without a pre-activation expansion and enrichment process. FIG. 4G shows flow cytometry plots of freshly thawed PBMCs showing gates for CD3+CD8+ cells and IFN-γ secretion signal using nanovials coated with CMV1 specific pMHC. FIG. 4H illustrates flow cytometry plots of the same PBMC population using tetramer staining. Gates for CD3+CD8-cells and tetramer positivity are shown.

FIGS. 5A-5I illustrate the discovery of rare functional TCRs using granzyme B secretion-based nanovial assay. FIG. 5A shows flow cytometry plots of granzyme B secretion specifically induced by pMHC-labeled nanovials at 3 hours for TCR156 transduced PBMCs loaded onto anti-CD45 labeled nanovials or PAP22 pMHC labeled nanovials. Secreting cells on pMHC-labeled nanovials sorted from the gated area are shown. Scale bars represent 50 μm. FIG. 5B shows flow cytometry plots of human donor PBMCs loaded onto nanovials and secreting granzyme B. CD3+CD8+ cells on nanovials were sorted as granzyme B secreting (Granzyme B+) or non-secreting cells (Granzyme B−). FIG. 5C is a summary of single-cell TCR αβ sequencing results. Sorted cells refers to the number of cells gated and sorted as positive or negative. FIG. 5D is anti-granzyme B barcode levels for Granzyme B+ and Granzyme B-populations as gated in FIG. 5B. Dashed lines represent the secretion barcode level cut-off: top Granzyme B^{High} (barcode>2000), middle Granzyme B^{Medium} (2000>barcode≥500). Granzyme B^{Low} (barcode<500). FIG. 5E shows the top 10 differentially expressed genes among Granzyme B+ and Granzyme B− populations. FIG. 5F shows the secretion phenotype distribution of Granzyme B+ clonotypes classified as Granzyme B^{High}, Granzyme B^{Medium} and Granzyme B^{Low}. FIG. 5G shows the discovery of three functional TCRs validated upon re-expression in human PBMCs and measurement of IFN-γ secretion following exposure to APCs with added peptides. IFN-γ secretion measurement of all 25 TCRs tested are represented in FIG. 14B. Two TCRs from the Granzyme B^{High} (right two) and one TCR from Granzyme B^{Medium} (leftmost bar) populations were found to be functional. FIG. 5H illustrates that nanovials accurately recovered the epitope information encoding specific pMHC molecules for the three functional TCRs in FIG. 5G. FIG. 5I illustrates the highest recovery rate of functional TCRs were observed from TCRs recovered based on the highest granzyme B secretion barcode signal.

FIGS. 6A-6E illustrate the multiplexed secretion-based profiling of prostate tissue antigen-specific T cells. FIG. 6A illustrates FACS analysis and sorting gates for identifying functional antigen-specific T cells transduced with TCR128 loaded on HLA-A*02:01 restricted PAP21 pMHC labeled nanovials. IFN-γ and TNF-α secretion signals were analyzed from the CD3⁺/CD8⁺/NGFR⁺ cells. Images of T cells on nanovials that were sorted reflecting each of the four quadrant gates including an IFN-γ and TNF-α polyfunctional population (Q2). Scale bars represent 50 μm. FIG. 6B illustrates the population distribution based on secretion phenotype is shown as a pie chart for TCR 128, 218, 156 transduced cells. CD3+CD8+ cells with secretion signal below the background threshold were considered as non-secretors. FIG. 6C is a schematic overview of multiplexed profiling of untransduced human primary T cells based on cytokine secretion and cell phenotype. T cells loaded on nanovials labeled with two cytokine capture antibodies (anti-IFN-γ and anti-TNF-α, or anti-IFN-γ and anti-IL-2) and anti-CD45 were activated under PMA/ionomycin stimulation. Secreted cytokines and cell surface markers (CD4. CD8) were stained with fluorescent detection antibodies, followed by analysis and sorting with a cell sorter. FIG. 6D shows the distribution of secretion phenotype for CD4+ and CD8+ human primary T cells based on IFN-γ and TNF-α secretion. FIG. 6E shows the distribution of secretion phenotype for CD4+ and CD8+ human primary T cells based on IFN-γ and IL-2 secretion.

FIGS. 7A-7C illustrate nanovial fabrication and functionalization. FIG. 7A shows an aqueous phase comprised of 4-arm-PEG Acrylate and photoinitiator is co-flowed with a gelatin solution in a microfluidic droplet generator. After droplet formation. PEG and gelatin undergo phase separation and are exposed to UV light to cross-link. After collection, nanovials are incubated with sulfo-NHS-biotin to be biotinylated. Localized fluorescence of AlexaFluor488-labeled streptavidin is observed in nanovial cavities. Scale bar represents 50 μm. FIG. 7B shows flow cytometry fluorescence histograms of nanovials following a dose-dependent cytokine capture assay with different capture antibody combinations. Nanovials were functionalized with anti-CD45 and with one or two cytokine capture antibodies (anti-IFN-γ, anti-TNF-α) and incubated with 0, 10, 100, or 1000 ng/ml of recombinant TNF-α or IFN-γ. The ability of nanovials to detect each individual cytokine was not significantly affected by the presence of other cytokine capture antibodies. FIG. 7C shows that fluorescence intensity is based on the concentration of pMHC incubated with nanovials. Nanovials were incubated with 0-100 μg/mL of pMHC monomers and stained with fluorescent anti-HLA-A2 antibodies followed by measurement of fluorescence intensity via SONY SH800 sorter. Signal of loaded pMHC increases linearly up to a concentration of ˜80 μg/mL of pMHC monomers. Scale bars represent 50 μm.

FIGS. 8A-8C show the optimization of human primary T cell loading. FIG. 8A shows the loading of human primary T cells into anti-CD45 and anti-IFN-γ labeled nanovials at different cell number to nanovial ratios. The highest fraction of single-cell loaded nanovials was achieved when cells were seeded at 1.6 cells per nanovial. Increased cell seeding density resulted in a larger fraction of nanovials with two or more T cells. Loading of cells into nanovial cavities followed Poisson statistics. Scale bars represent 50 μm. FIG. 8B illustrates the dependence of cell binding on surface marker target and antibody concentration. Left: Nanovials conjugated with anti-CD45 showed the highest loading efficiency when primary T cells were loaded after initial CD3/CD28 activation in culture. Right: Increased anti-CD45 concentration on nanovials increased cell binding by nearly 6-fold. FIG. 8C shows the effect of cytokine capture antibodies on cell loading. Flow cytometry histograms of cells (calcein AM positive) loaded on nanovials in the presence of anti-IFN-γ or anti-TNF-α antibodies.

FIGS. 9A-9C illustrate the selection of viable T cells based on cytokine secretion. FIG. 9A shows the gates for isolating cells on nanovials with secretion signal. Using a SONY SH800S, the nanovial population was identified in FSC/SSC and further gated for high calcein AM signal. Secretion signal was quantified for this sub-population using the fluorescence peak area vs. peak height (A/H). Single cells were sorted as high, medium, and low secretors based on IFN-γ and TNF-α secretion level (see FIG. 9B). FIG. 9B further shows fluorescence microscopy images of sorted high, medium, and low secretors. Dotted lines in insets outline the nanovial boundaries. Scale bars represent 50 μm. FIG. 9C illustrates crosstalk between nanovials. Two nanovial types were introduced together to evaluate crosstalk. Fluorescently labeled nanovials (AlexaFluor 488) without cells were mixed with T cell-loaded nanovials activated with PMA/ionomycin at a ratio of 1:2. Secretion signal was labeled and analyzed on both nanovial types by first gating on the green fluorescence signal on nanovials (Alexa 488 Area). Scatter plots of the cell-loaded nanovial population shows a larger population with high IFN-γ secreting cells compared to nanovials that were not loaded with cells. The percent of cells in the identical gated regions are shown. Scale bars represent 100 μm.

FIGS. 10A-10D shows the optimization of antigen-specific T cell loading, secretion on nanovials and expansion of cells post-isolation. FIG. 10A illustrates the analysis of antigen-specific T cell loading as a function of pMHC concentrations on nanovials. Top flow cytometry histograms show the fraction of untransduced cell loaded nanovials based on calcein AM signal. Bottom shows the fraction of 1G4-transduced cell nanovials and corresponding NGFR positivity of those bound cells (dashed line). FIG. 10B shows flow cytometry fluorescence histograms of nanovials following loading of IG4-transduced PBMCs on pMHC-labeled nanovials. Histograms for IFN-γ secretion are shown as a function of time (0, 3, 6, 12 hours). FIG. 10C shows images showing expansion of 1G4-transduced T cells following sorting and detachment with collagenase D treatment. Microscopy images show cells expanded in culture over 5 days. FIG. 10D shows flow cytometry fluorescence histograms of viability and NGFR levels for the expanded population expressing the 1G4 TCR on Day 5. Scale bars represent 50 μm.

FIGS. 11A-11B show the recovery of cognate T cells with various affinities to HLA-A*02:01 restricted NY-ESO-1 pMHC are shown. FIG. 11A shows the flow cytometry plots of isolating 1G4-transduced PBMCs or untransduced cells loaded onto NY-ESO-1 pMHC labeled nanovials. Enriched cells were defined based on gates on viability (calcein AM), CD3 and CD8, and IFN-γ secretion. The NGFR fraction above the background was also determined using a gate on PE-Cy7 area. FIG. 11B shows flow cytometry plots of isolating 1G4-transduced PBMCs or untransduced cells based on dual-color MHC tetramer signal. Raw numbers of recovered cognate T cells with various affinities using pMHC-labeled nanovials or dual-color tetramers are shown in Table 1.

FIGS. 12A-12C illustrate FACS analysis and gating strategy for isolation of cognate T cells using nanovials, tetramers, or CD137 activation markers. FIG. 12A shows flow scatter plots showing gates for selecting viable CD8+ T cells binding to nanovials and secreting IFN-γ. CD137+CD8+ T cells, and tetramer+ CD8+ T cells. Microscopy images show representative sorted cells on nanovials. Scale bar represents 50 μm. FIG. 12C shows scatter plots show calcein AM fluorescence Peak Area vs. Peak Width gates used for isolating single cells on nanovials. Images of sorted events show that the population with a similar peak area but with a shorter peak width contains >90% single cells loaded on nanovials. Data on single-cell occupancy is shown in a bar graph. Scale bars represent 50 μm. FIG. 12C shows the distribution of cell clonotypes recovered by nanovials with matching target pMHC information: 32 CMV1 (Clonotypes-0) through Clonotype-148), 1 CMV2 (Clonotype_177), 12 EBV specific clonotypes (Clonotypes_3 through Clonotype_213) in FIG. 12C.

FIGS. 13A-13D show the determination of the dominant epitope for each clonotype from the number of barcodes detected. For clonotypes matched with more than one barcode (clonotype0, 1, 2, 3, 4, 6), the final epitope was determined based on the barcode with the highest fraction, representing >90% of cells represented.

FIGS. 14A-14B show linking cell secretion to recovered TCRs and their functional validation. FIG. 14A illustrates how clonotypes were ranked based on granzyme B secretion barcode signal (right, HS-P1 to HS-P6) or frequency of recovered TCR sequence (left, black, HF-P1 to HF-P15). Each clonotype was classified as Granzyme B^{High} (identified dots). Granzyme B^{Medium} (light dots) or Granzyme B^{Low} (dark dots) based on their secretion level (FIG. 5D). FIG. 14B illustrates re-expression of each candidate TCR sequence in human PBMCs and measurement of IFN-γ secretion following exposure to antigen presenting cells with exogenously added cognate peptides: peptide pool (terminated with square on bars) or single-peptide obtained from nanovial barcode encoding the specific pMHC molecule (terminated with triangle on bars). As a negative control, each TCR-transduced PBMCs were co-cultured with antigen presenting cells with DMSO (terminated with circle on bars).

FIGS. 15A-15F shows detailed FACS analysis and gating strategy for multiplexed secretion-based profiling using nanovials. FIG. 15A illustrates flow cytometry dot plots with gating defining populations positive for IFN-γ and TNF-α secretion on nanovials with single cytokine capture antibodies. FIG. 15B illustrates flow cytometry plots for identifying functional antigen-specific T cells transduced with TCR218 and TCR156 loaded on HLA-A*02:01 restricted PAP21 and PAP22 pMHC labeled nanovials, respectively. IFN-γ and TNF-α secretion signals were analyzed from the calcein AM stained, CD3 and CD8 double positive population with NGFR signal above background. FIG. 15 illustrates control flow cytometry plots for analyzing secretions from TCR156 transduced PBMCs loaded on non-cognate PAP14 pMHC labeled nanovials or from untransduced PBMCs loaded on PAP21 and PAP22 pMHC labeled nanovials (FIG. 15D. FIGS. 15E and 15F illustrates multiplexed secretion profiling of untransduced human primary T cells activated by PMA/Ionomycin. Experimental dot plots showing IFN-γ and TNF-α or IFN-γ (FIG. 15E) and IL-2 (FIG. 15F) secretion from activated CD4+ and CD8-T cells loaded on nanovials. Single cells were sorted based on CD4+ or CD8+ gates as well as the four quadrant gates. Scale bars represent 50 μm.

FIGS. 16A-16D illustrates detailed list of clonotypes recovered from nanovials (frequency >=2) with corresponding V(D)J αβ genes. CDR3 αβ sequences, frequency of clonotype and epitope information.

FIGS. 17A-17D illustrate how fluorescence peak area and height were used to measure single-cell secretions on nanovials. FIG. 17A is an overview schematic of the nanovial fluorescence peak shape obtained when a nanovial transits a laser spot in a flow cytometer. FIG. 17B illustrates fluorescence microscopy images of pre-sort nanovials with cells showing two distinct fluorescence patterns, with dotted lines in insets outlining the nanovial boundaries: fluorescence spread across the nanovial cavity from secreted cytokines or fluorescence associated with cells on nanovials without signal across the cavity area. The fluorescence intensity profile was computed across the cavity of each image and the maximum intensity (height, H), area under the intensity curve (area, A), and the ratio between the area and height were calculated. Scale bars represent 100 μm. FIG. 17C shows isolation of T cells on nanovials with secretion signal. Three different gates were used to differentiate spatially-extended IFN-γ secretion signals on nanovials (low secretion and high secretion gates) from signal solely from non-specific cell surface binding (label binding to cell gate). Sorted cells on nanovials have different distribution of fluorescence signal, as shown in the images. Scale bars represent 50 μm. FIG. 17D illustrates nanovials with each cytokine (TNF-α or IL-2) secretion signal were sorted using area vs. height metrics. The ability to isolate on-nanovial cytokine staining was consistent across different cytokines as shown in fluorescence microscopy images. Scale bars represent 50 μm.

FIG. 18 illustrates the amino acid sequence of TRAV (alpha) and TRBV (beta) amino acids in the different TCRs listed therein.

FIG. 19 illustrates the amino acid sequence of TRAV (alpha) and TRBV (beta) amino acids in the different TCRs listed therein. These three TCRs were identified as functional TCRs that are prostate cancer specific.

### DETAILED DESCRIPTION OF ILLUSTRATED EMBODIMENTS

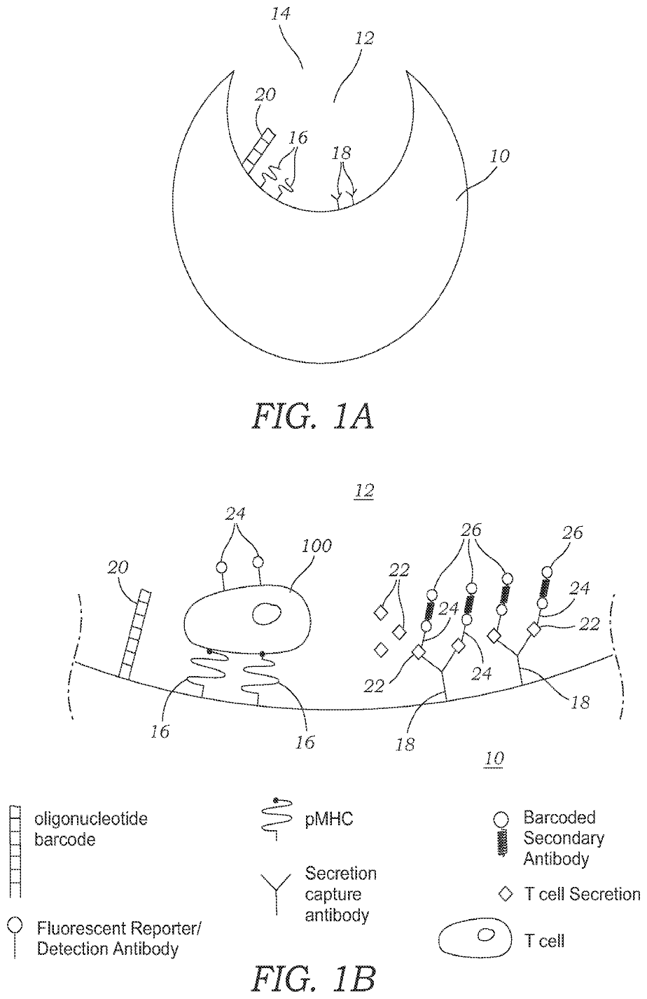

With reference to FIGS. 1A and 1B, the three-dimensional shaped particles 10 (also referred to herein as nanovials) are typically micrometer sized particles. Generally, the three-dimensional shaped particles 10 have a longest dimensional length of around 100 μm or less. For applications that require the loading of cells 100 (FIG. 1B) into/onto the three-dimensional shaped particles 10, the three-dimensional shaped particles 10 typically have a minimum dimensional length of at least 10 μm. In embodiments in which flow cytometers or fluorescence activated cell sorters are used to analyze or sort the three-dimensional shaped particles 10 which contain the cells 100, the three-dimensional shaped particles 10 are preferably between ˜30 μm and ˜60 μm in a maximum dimension (here the three-dimensional shaped particles 10 with an average outer diameter of 35 μm were formed). The three-dimensional shaped particles 10 may be formed from biocompatible materials or polymers. The materials or polymers are typically transparent to visible light. In one embodiment, the three-dimensional shaped particles 10 are formed from polyethylene glycol (PEG).

The three-dimensional shaped particles 10 include a cavity 12 as best seen in FIG. 1A. The cavity 12 may have an opening 14 that opens to the external environment of the three-dimensional shaped particle 10 as illustrated in FIG. 1A. The opening of the cavity 12 is dimensioned to allow cells 100 (and in one preferred embodiment T cells) to enter the cavity 12. The three-dimensional shaped particles 10 may have a cavity 12 sized to fit a single cell 100. For example, the three-dimensional shaped particles 10 may have a cavity 12 with a longest dimension of 10 μm-30 μm. In a preferred embodiment, the cavity 12 is dimensioned to hold a sub-nanoliter volume of fluid. The fluid may include an aqueous-based fluid. As explained herein, in some preferred embodiments, the three-dimensional shaped particles 10 preferably are designed to carry or hold cell(s) 100 and in particular T cells within the cavity 12. The cell(s) 100 may be located within the volume of fluid within the void or cavity. As explained herein, the T cell(s) 100 may adhere or become adherent to a surface of the three-dimensional shaped particle 10 within the cavity 12 by adhering to peptide-major histocompatibility complex (pMHC) monomers 16 that are disposed on a surface of the three-dimensional shaped particle 10 within the cavity 12. Each three-dimensional shaped particle 10 or nanovial may have tens to hundreds of millions of pMHC molecules 16 within the cavity 12 to capture T cells 100 with high avidity and activate cells with cognate TCRs. The number of pMHC molecules 16 disposed on the three-dimensional shaped particle 10 surface may be tuned to lower or higher levels to allow binding of higher or lower affinity TCRs. For example, the number of pMHC monomers/molecules 16 could be dosed to 100,000-500,000 to have less avidity effects and only capture T cells 100 with higher affinity TCRs, or TCRs with reduced kinetic off rates.

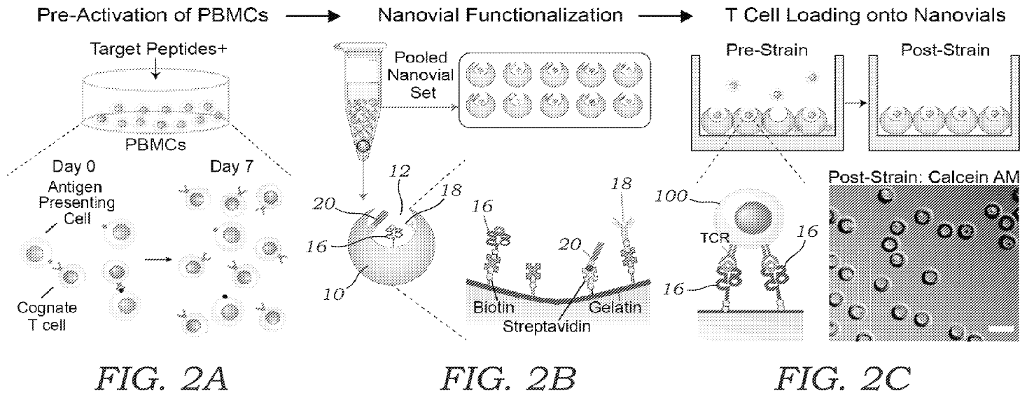

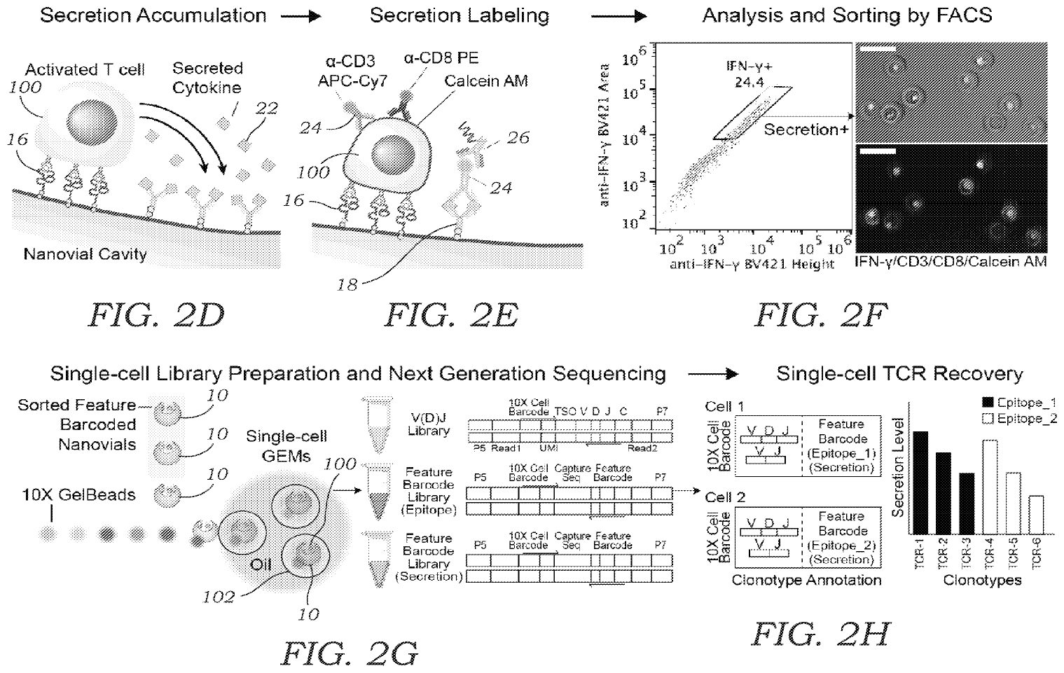

The three-dimensional shaped particles 10 or nanovials preferably have the inner cavity 12 functionalized with biotin during fabrication to enable linkage of multiple biotinylated antibodies or peptide-MHC (pMHC) 16 monomers with epitopes of interest through streptavidin-biotin noncovalent interactions. The three-dimensional shaped particles 10 have secretion capture antibodies 18 bound or linked to the surface of the cavity 12. The secretion capture antibodies 18 capture biomolecules secreted from the T cells 100 that are bound to the pMHC molecules 16 (FIG. 2D). In one particular embodiment for the capture and secretion analysis of primary human T cells 100, irrespective of antigen targeting, nanovials 10 may be decorated with secretion capture antibodies 18 that include biotinylated anti-CD45 and cytokine capture antibodies against interferon-γ, tumor necrosis factor-α, and interleukin-2 (anti-IFN-γ, anti-TNF-α, anti-IL 2). Other cytokines, growth factors, or secreted products may also be captured using appropriate secretion capture antibodies 18. For example, regulatory T cells can be identified by secretion of IL-10, IL-35, and/or TGF-β.

FIG. 1A illustrates a three-dimensional shaped particle 10 or nanovial with pMHC molecules 16 on the inner surface of the cavity 12. The cavity 12 is also populated with secretion capture antibodies 18 and an oligonucleotide barcode 20 linked thereto via streptavidin-biotin chemistry. FIG. 1A illustrated the state of the three-dimensional shaped particle 10 prior to loading of a T cell 100 into the cavity 12.

By having the cavity 12 of the three-dimensional shaped particle 10 or nanovial populated with pMHC monomers 16 single antigen-specific T cell secretion assays can be performed. FIG. 1B illustrates how such T cell secretion assays may be performed using the three-dimensional shaped particles 10. As seen in FIG. 1B, the T cells 100 that are located in the cavity 12 of the three-dimensional shaped particle 10 bind to the pMHC molecules 16 and secrete or release biomolecules as secretions 22 and are captured with secretion capture antibodies 18. The secretions 22 may be further labeled, e.g., with fluorescent reporters 24 or detection antibody to characterize the amount, affinity, specificity, or other properties of the secretions 22 (FIGS. 1B and 2E). In one embodiment, and with reference to FIGS. 1B and 2E a barcoded secondary antibody 26 is provided to bind to the secretions 22 to allow encoding of secretion levels (e.g., cytokine secretion) into the single-cell sequencing data set and ranking of TCR sequences based on the amount of secretions 22 secreted. In a related embodiment, the primary secretion detection antibody or fluorescent reporter 24 is directly labeled with a barcode. The barcoded fluorescent reporter or labelled antibody may thus bind directly to the secretion 22 or indirectly through a primary fluorescent reporter/detection antibody 24. As noted herein, barcoding of the amount of secretion improves the ability to detect rare TCRs with high confidence. Thus, the secretions 22 (e.g., cytokines) can be labeled with oligonucleotide-labeled antibodies 26 to enable workflows in which TCR sequences are linked to the amount of secretions 22 using single-cell RNA-sequencing instruments and reagents as described herein. The captured T cells 100 may also be labeled, for example, with fluorescent reporters 24 or detection antibodies, or labeled with dyes. Fluorescent reporter(s) 24 specific to the secretion capture antibodies 18, a T cell receptor, or a surface marker may be used. Fluorescent reporter(s) 24 may also include viability dyes. FIG. 2E illustrates a T cell 100 labeled with calcein AM, Anti-CD3 APC Cy7, and anti-CD8 PE. Other surface markers can be used to detect specific cell types (e.g., CD4, CD25) or activation state of the T cells 100, such as CD69 or CD137 to further refine the analysis and/or recovery of TCRs to specific cell sub-populations.

In one embodiment, the three-dimensional shaped particles 10 or nanovials have unique pMHC monomers 16 loaded within the cavity 12 (FIG. 2B). That is to say, in this particular embodiment, each three-dimensional shaped particle 10 or nanovial has only one type of pMHC monomer 16 populating the surface cavity 12 to capture T cells 100. A plurality of different three-dimensional shaped particles 10 may then comprise unique pMHC monomers 16, comprising separate displayed peptides and/or separate HLA-types. In this embodiment, the different three-dimensional shaped particles 10 comprising unique pMHC monomers 16, also comprise a unique oligonucleotide barcode 20 specific to the unique pMHC monomer 16. Of course, in other embodiments, different three-dimensional shaped particles 10 have the same pMHC monomers 16 loaded therein. Secretion capture antibodies 18 are also present within the cavity 12 to capture secretions 22 from T cells 100. In addition, the shaped three-dimensional particles 10 or nanovials are labelled within unique oligonucleotide-barcodes 20 so that one can link a particular TCR sequence to cognate pMHC 16 with 100% accuracy.

In one embodiment, the TCRs of sorted T cells 100 on nanovials are sequenced, recovering paired TCR αβ-chains using microfluidic emulsion-based single-cell sequencing. In one embodiment. TCRs are sequenced using the commercially available 10× Chromium Next GEM Chip K as described herein. This allows the recovery and characterization of clonotypes recovered from the three-dimensional shaped particles 10 or nanovials with corresponding V(D)J αβ genes. CDR3 αβ sequences, frequency of clonotype and epitope information from the linked unique oligonucleotide barcode 20. In addition, the TCR workflow includes the ability to link secretion levels within the nanovials with the barcoded secondary antibody 26.

Recovered TCR sequences specific to a target antigen can then be engineered into new (engineered) T cells from a patient for therapeutic uses, e.g., using a pMSGV retroviral plasmid, lentivirus. CRISPR-Cas9 gene insertion, or related viral introduction or genome editing technologies. The engineered T cells may include autologous T cells or allogeneic T cells. Engineered T cells can be expanded ex vivo or in vivo to treat a specific disease, such as viral infection, cancer, or other conditions which can benefit from selective cell killing. Specifically, cytomegalovirus (CMV) and Epstein-Barr virus (EBV)-reactive TCRs disclosed herein can be applied to treat CMV infection after stem cell transplantation or EBV-caused head and neck carcinoma. The engineered T cells may be used therapeutically to treat prostate cancer using rare TCRs with activity against prostate cancer-specific antigens. Alternatively, regulatory T (Treg) cells with engineered TCRs specific to cells of a tissue being attacked by the immune system can be used to protect from autoimmunity or transplant rejection.

As noted above, the nanovial technology disclosed herein has been utilized to isolate T cell receptors that recognize a number of different cellular and viral polypeptides. In this context, embodiments of the invention include compositions of matter comprising, for example, one or more vectors comprising the TCR polynucleotides disclosed herein and methods for making and using such compositions. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to. Sendai viral vectors, adenoviral vectors, adeno-associated virus vectors, retroviral vectors, lentiviral vectors, and the like.

Typically, the vector is an expression vector. The term “expression” as used herein is defined as the transcription and/or translation of a particular nucleotide sequence driven by its promoter. In this context, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

Typically, a composition of the invention comprises one or more Vα/Vβ polynucleotides, for example a polynucleotide encoding a TCR Vα polypeptide in combination with a polynucleotide encoding a TCR Vβ polypeptide such that a Vα/Vβ TCR can be expressed on the surface of a mammalian cell (e.g., a CD8⁺ T cell) transduced with the vector(s), wherein the Vα/Vβ TCR recognizes a peptide antigen (e.g., a MEAF6, SCAMP3, CMV or EBV peptide antigen) associated with a HLA. The term “transduced” or “transfected” or “transformed” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As discussed below, in one aspect, the invention includes a method for generating a modified T cell comprising introducing one or more exogenous nucleic acids (e.g., nucleic acids disposed within a lentiviral vector) encoding a TCR disclosed herein into a T cell (e.g., a CD8⁺ T cell obtained from an individual diagnosed with a cancer that expresses a polypeptide epitope recognized by the TCR). The present invention also includes modified T cells with downregulated or knocked out gene expression (e.g., a modified T cell having a knocked out endogenous T cell receptor and an exogenous/introduced T cell receptor that recognizes a peptide associated with a HLA). The term “knockdown” as used herein refers to a decrease in gene expression of one or more genes. The term “knockout” as used herein refers to the ablation of gene expression of one or more genes.

The modified T cells described herein may be included in a composition for use in a therapeutic regimen. The composition may include a pharmaceutical composition and further include a pharmaceutically acceptable carrier. A therapeutically effective amount of the pharmaceutical composition comprising the modified T cells may be administered. Pharmaceutical compositions of the present invention may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Adoptive immunotherapy with T cells harboring antigen-specific TCRs have therapeutic potential in the treatment of cancers and other diseases. Gene-engineering of CD8⁺ T cells with a specific TCR has the advantage of redirecting the T cell to a selected antigen such as a polypeptide epitope recognized by a TCR. In this context, in one aspect, the invention includes methods for stimulating a T cell-mediated immune response to a target cell or tissue in a subject comprising administering to a subject an effective amount of a modified CD8⁺ T cell. In this embodiment, the CD8⁺ T cell is modified as described elsewhere herein. Embodiments of the invention also include administering multiple modified CD8⁺ T cells that target multiple polypeptide epitopes. For example, embodiments of the invention include administering at least two different modified CD8⁺ T cells, for example a first modified CD8⁺ T cell that targets a MEAF6 or SCAMP3 peptide associated with a first human leukocyte antigen in combination with a second CD8⁺ T cells that targets a MEAF6 or SCAMP3 peptide associated with second human leukocyte antigen.

Illustrative embodiments of the invention include compositions of matter comprising a polynucleotide encoding a T cell receptor (TCR) alpha chain polypeptide and/or a TCR beta chain polypeptide; wherein the polynucleotide is disposed in a vector, and when the vector is transduced into a CD8⁺ T cell, the alpha chain polypeptide and/or the TCR beta chain polypeptide encoded by the polynucleotide form a T cell receptor that recognizes a polypeptide epitope present in a human MYST/Esa1 associated factor 6 (MEAF6) splicing variant polypeptide (MEAF6 is NCBI Reference Sequence: NM_001270875.3; see also Lee et al., Proc Natl Acad Sci USA 100 (5), 2651-2656 (2003)).

In some embodiments of the invention, the T cell receptor recognizes a polypeptide epitope present in a human MYST/Esa1 associated factor 6 (MEAF6) splicing variant polypeptide in combination with a human leukocyte antigen HLA-A. In certain embodiments of the invention, the T cell receptor recognizes a polypeptide epitope present in: SGMFDYDFEYV (SEQ ID NO: 131) or GMFDYDFEYV (SEQ ID NO: 135). In some embodiments of the invention, the polynucleotide encodes a segment of at least 10, 25 or 50 amino acids having an at least 98% sequence identity to a segment of amino acids in the alpha and/or the beta chain of NVTCR21 (SEQ ID NO: 121 and/or SEQ ID NO: 124). In certain embodiments of the invention, the polynucleotide encodes amino acids of a TCR variable region, and the vector comprises vector polynucleotides encoding a TCR constant region fused in frame with the TCR variable region.

In certain embodiments of the invention, polynucleotide encoding a TCR disclosed herein is disposed within a cell (e.g., a human leukocyte cell). For example, in an illustrative embodiment of the invention, the cell is a CD8⁺ T cell obtained from an individual diagnosed with a cancer that expresses a human MEAF6 splicing variant; and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a MEAF6 splicing variant peptide associated with a human leukocyte antigen expressed on the surface of cells of the cancer.

Embodiments of the invention also include methods of inhibiting growth of a cancer cell (e.g., a prostate cancer cell or lung cancer cell), the methods comprising combining the cancer cell with a CD8⁺ T cell transduced with a polynucleotide encoding a T cell receptor (TCR) alpha chain polypeptide and a TCR beta chain polypeptide; wherein when transduced into and expressed in the CD8⁺ T cell, the alpha chain polypeptide and the TCR beta chain polypeptide can form a T cell receptor that recognizes a polypeptide epitope present in a human MYST/Esa1 associated factor 6 (MEAF6) splicing variant expressed on the cancer cell, thereby inhibiting growth of the cancer cell.

Embodiments of the invention also include compositions comprising a polynucleotide encoding a T cell receptor (TCR) alpha chain polypeptide and/or a TCR beta chain polypeptide; wherein the polynucleotide is disposed in a vector, and when the vector is transduced into a CD8⁺ T cell, the TCR alpha chain polypeptide and/or the TCR beta chain polypeptide encoded by the polynucleotide form a T cell receptor on the CD8⁺ T cell that recognizes a polypeptide epitope present in a human secretory carrier-associated membrane protein 3 (SCAMP3) polypeptide (SCAMP3 is Gene ID: 10067; see also Thomas P. et al. Biochem Biophys Res Commun. 2016 Sep. 23. PMID 27507217).

In some embodiments of the invention, the T cell receptor recognizes a polypeptide epitope present in STMYYLWML (SEQ ID NO: 133). In certain embodiments of the invention, the T cell receptor recognizes a polypeptide epitope of SCAMP3 in combination with a human leukocyte antigen HLA-A*02:01. In some embodiments of the invention, the polynucleotide encodes amino acids of a TCR variable region, and the vector comprises vector polynucleotides encoding a TCR constant region fused in frame with the TCR variable region. In certain embodiments of the invention, the polynucleotide encodes a segment of at least 10, 25 or 50 amino acids of a TCR variable region having an at least 98% sequence identity to a segment of amino acids the alpha and/or the beta chain of NVTCR11 (SEQ ID NO: 119 and/or SEQ ID NO: 122); or NVTCR19 (SEQ ID NO: 120 and/or SEQ ID NO: 123).

In certain embodiments of the invention, polynucleotide encoding a TCR disclosed herein is disposed within a cell (e.g., a human leukocyte cell). For example, in some embodiments of the invention, the cell is a CD8⁺ T cell obtained from an individual diagnosed with a cancer that expresses a human SCAMP3 polypeptide; and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a SCAMP3 polypeptide associated with a human leukocyte antigen expressed on the surface of cells of the cancer.

Embodiments of the invention also include methods of inhibiting growth of a cancer cell (e.g., a breast cancer cell, a glioma cell or a hepatocarcinoma cell) comprising combining the breast cancer cell, the glioma cell or the hepatocarcinoma cell with a CD8⁺ T cell transduced with a polynucleotide encoding a T cell receptor (TCR) alpha chain polypeptide and a TCR beta chain polypeptide; wherein when transduced into and expressed in the CD8⁺ T cell, the alpha chain polypeptide and the TCR beta chain polypeptide can form a T cell receptor that recognizes a polypeptide epitope present in a SCAMP3 polypeptide expressed on the cancer cell.

Embodiments of the invention also include compositions of matter comprising a polynucleotide encoding a T cell receptor alpha chain polypeptide and/or a TCR beta chain polypeptide; wherein the polynucleotide is disposed in a vector, and when the vector is transduced into a CD8⁺ T cell, the TCR alpha chain polypeptide and/or the TCR beta chain polypeptide encoded by the polynucleotide form a T cell receptor on the CD8⁺ T cell that recognizes a polypeptide epitope present in a cytomegalovirus (CMV) polypeptide. In some embodiments of the invention, the T cell receptor recognizes a polypeptide epitope present in NLVPMVATV (SEQ ID NO 2) or VLEETSVML (SEQ ID NO 3). In some embodiments of the invention, the T cell receptor recognizes a polypeptide epitope of CMV in combination with a human leukocyte antigen HLA-A*02:01. In some embodiments of the invention, the polynucleotide encodes amino acids of a TCR variable region, and the vector comprises vector polynucleotides encoding a TCR constant region fused in frame with the TCR variable region. In certain embodiments of the invention, the polynucleotide encodes a segment of at least 10, 25 or 50 amino acids amino acids of a TCR variable region having an at least 98% sequence identity to a segment of amino acids the alpha and/or the beta chain of TCR1 (SEQ ID NO: 55 and/or SEQ ID NO: 87), TCR2 (SEQ ID NO: 56 and/or SEQ ID NO: 88), TCR3 (SEQ ID NO: 57 and/or SEQ ID NO: 89), TCR4 (SEQ ID NO: 58 and/or SEQ ID NO: 90), TCR5 (SEQ ID NO: 59 and/or SEQ ID NO: 91), TCR6 (SEQ ID NO: 60 and/or SEQ ID NO: 92), TCR8 (SEQ ID NO: 62 and/or SEQ ID NO: 94), TCR9 (SEQ ID NO: 63 and/or SEQ ID NO: 95), TCR12 (SEQ ID NO: 66 and/or SEQ ID NO: 98), TCR13 (SEQ ID NO: 67 and/or SEQ ID NO: 99), TCR14 (SEQ ID NO: 68 and/or SEQ ID NO: 100), TCR15 (SEQ ID NO: 69 and/or SEQ ID NO: 101), TCR17 (SEQ ID NO: 71 and/or SEQ ID NO: 103), TCR21 (SEQ ID NO: 75 and/or SEQ ID NO: 107), TCR22 (SEQ ID NO: 76 and/or SEQ ID NO: 108), TCR23 (SEQ ID NO: 77 and/or SEQ ID NO: 109), TCR24 (SEQ ID NO: 78 and/or SEQ ID NO: 110), or TCR25 (SEQ ID NO: 79 and/or SEQ ID NO: 111).

In certain embodiments of the invention, the polynucleotide is disposed in a cell, for example a CD8⁺ T cell obtained from an individual having undergone a stem cell transplantation and diagnosed with a CMV infection; and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a CMV polypeptide associated with a human leukocyte antigen expressed on the surface of cells infected with CMV.

Embodiments of the invention also include methods of inhibiting cytomegalovirus (CMV) growth comprising combining a human cell infected with CMV with a CD8⁺ T cell transduced with a polynucleotide encoding a T cell receptor alpha chain polypeptide and a TCR beta chain polypeptide; wherein when transduced into and expressed in the CD8⁺ T cell, the alpha chain polypeptide and the TCR beta chain polypeptide can form a T cell receptor that recognizes a polypeptide epitope present in a cytomegalovirus, thereby inhibiting growth of the cytomegalovirus.

Embodiments of the invention also include compositions of matter comprising a polynucleotide encoding a T cell receptor alpha chain polypeptide and/or a TCR beta chain polypeptide; wherein the polynucleotide is disposed in a vector, and when the vector is transduced into a CD8⁺ T cell, the TCR alpha chain polypeptide and/or the TCR beta chain polypeptide encoded by the polynucleotide form a T cell receptor on the CD8⁺ T cell that recognizes a polypeptide epitope present in a Epstein Barr virus (EBV) polypeptide. In some embodiments of the invention, the T cell receptor recognizes a polypeptide epitope present in GLCTLVAML (SEQ ID NO 4). In some embodiments of the invention, the T cell receptor recognizes a polypeptide epitope of EBV in combination with a human leukocyte antigen HLA-A*02:01. In some embodiments of the invention, the polynucleotide encodes amino acids of a TCR variable region, and the vector comprises vector polynucleotides encoding a TCR constant region fused in frame with the TCR variable region. In certain embodiments of the invention, the polynucleotide encodes a segment of at least 10, 25 or 50 amino acids amino acids of a TCR variable region having an at least 98% sequence identity to a segment of amino acids the alpha and/or the beta chain of TCR7 (SEQ ID NO: 61 and/or SEQ ID NO: 93). TCR10 (SEQ ID NO: 64 and/or SEQ ID NO: 96), TCR11 (SEQ ID NO: 65 and/or SEQ ID NO: 97), TCR16 (SEQ ID NO: 60 and/or SEQ ID NO: 70), TCR18 (SEQ ID NO: 60 and/or SEQ ID NO: 72), TCR19 (SEQ ID NO: 73 and/or SEQ ID NO: 105), TCR20 (SEQ ID NO: 74 and/or SEQ ID NO: 106), or TCR32 (SEQ ID NO: 86 and/or SEQ ID NO: 118).

In certain embodiments of the invention, the polynucleotide is disposed in a cell, for example a CD8⁺ T cell obtained from an individual diagnosed with a head carcinoma or a neck carcinoma; and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a EBV polypeptide associated with a human leukocyte antigen expressed on the surface of cells infected with EBV.

Embodiments of the invention also include methods of inhibiting Epstein Barr virus (EBV) growth comprising combining a human cell infected with EBV with a CD8⁺ T cell transduced with a polynucleotide encoding a T cell receptor alpha chain polypeptide and a TCR beta chain polypeptide; wherein when transduced into and expressed in the CD8⁺ T cell, the alpha chain polypeptide and the TCR beta chain polypeptide can form a T cell receptor that recognizes a polypeptide epitope present in a Epstein Barr virus, thereby inhibiting growth of the Epstein Barr virus.

As noted above, in certain embodiments of these compositions, the polynucleotide encodes amino acids of a TCR variable region and the vector comprises vector polynucleotides encoding a TCR constant region fused in frame with the TCR variable region (see, e.g. U.S. Patent Publication Nos. 20220354889, 20200138865, 20210363245 and 20210155941; and Coren et al., Biotechniques. 2015 Mar. 1; 58(3):135-9 (which describes aspects of the MSGV Hu Acceptor vector sold by Addgene™). Typically, in these composition the polynucleotide is disposed in a cell (e.g., a human CD8⁺ T cell). Optionally, for example, the polynucleotide is disposed in a CD8⁺ T cell is obtained from an individual diagnosed with a cancer that expresses a MEAF6 or SCAMP3 peptide antigen (e.g., a prostate cancer); and the CD8⁺ T cell is transduced with a vector comprising a polynucleotide encoding a TCR Vα polypeptide in combination with a polynucleotide encoding a TCR Vβ polypeptide such that a heterologous TCR is expressed on a surface of the CD8⁺ T cell, wherein the heterologous TCR recognizes a MEAF6 or SCAMP3 peptide associated with a human leukocyte antigen expressed on the surface of cells of the cancer.

In certain compositions of the invention, the polynucleotide encodes a segment of at least 5, 10, 25, 50 or 100 amino acids of a TCR polypeptide embodiment of the invention disclosed herein (e.g., at least 5 or 10 amino acids present in an Alpha CDR1 polypeptide sequence, an Alpha CDR2 polypeptide sequence, an Alpha CDR3 polypeptide sequence, a Beta CDR1 polypeptide sequence, a Beta CDR2 polypeptide sequence or a Beta CDR3 polypeptide sequence). In certain compositions of the invention, the polynucleotide encodes a segment of at least 5, 10, 25, 50 or 100 amino acids having an at least 98% sequence identity to a segment of amino acids the alpha and/or the beta chain of NVTCR21 (SEQ ID NO: 121 and/or SEQ ID NO: 124), NVTCR11 (SEQ ID NO: 119 and/or SEQ ID NO: 122); or NVTCR19 (SEQ ID NO: 120 and/or SEQ ID NO: 123); TCR1 (SEQ ID NO: 55 and/or SEQ ID NO: 87), TCR2 (SEQ ID NO: 56 and/or SEQ ID NO: 88), TCR3 (SEQ ID NO: 57 and/or SEQ ID NO: 89), TCR4 (SEQ ID NO: 58 and/or SEQ ID NO: 90), TCR5 (SEQ ID NO: 59 and/or SEQ ID NO: 91), TCR6 (SEQ ID NO: 60 and/or SEQ ID NO: 92), TCR8 (SEQ ID NO: 62 and/or SEQ ID NO: 94), TCR9 (SEQ ID NO: 63 and/or SEQ ID NO: 95), TCR12 (SEQ ID NO: 66 and/or SEQ ID NO: 98), TCR13 (SEQ ID NO: 67 and/or SEQ ID NO: 99), TCR14 (SEQ ID NO: 68 and/or SEQ ID NO: 100), TCR15 (SEQ ID NO: 69 and/or SEQ ID NO: 101), TCR17 (SEQ ID NO: 71 and/or SEQ ID NO: 103), TCR21 (SEQ ID NO: 75 and/or SEQ ID NO: 107). TCR22 (SEQ ID NO: 76 and/or SEQ ID NO: 108), TCR23 (SEQ ID NO: 77 and/or SEQ ID NO: 109), TCR24 (SEQ ID NO: 78 and/or SEQ ID NO: 110), TCR25 (SEQ ID NO: 79 and/or SEQ ID NO: 111); TCR7 (SEQ ID NO: 61 and/or SEQ ID NO: 93), TCR10 (SEQ ID NO: 64 and/or SEQ ID NO: 96), TCR11 (SEQ ID NO: 65 and/or SEQ ID NO: 97), TCR16 (SEQ ID NO: 60 and/or SEQ ID NO: 70), TCR18 (SEQ ID NO: 60 and/or SEQ ID NO: 72), TCR19 (SEQ ID NO: 73 and/or SEQ ID NO: 105), TCR20 (SEQ ID NO: 74 and/or SEQ ID NO: 106), or TCR32 (SEQ ID NO: 86 and/or SEQ ID NO: 118) (as is known in the art, sequence identity is the ratio of the number of identical amino acids between the 2 aligned sequences/segments over the aligned length, expressed as a percentage). In some embodiments of the invention, the T cell receptor alpha chain polypeptide and/or the TCR beta chain polypeptide encoded by the polynucleotide comprises an amino acid substitution mutation of the wild type TCR amino acid sequence, such as one selected to optimize its interaction with its cognate ligand (see, e.g. Sibener et al., Cell 174, 672-687, Jul. 26, 2018; and Zhao et al., Science 376, 155 (2022), the contents of which are incorporated herein by reference).

In another aspect, the invention includes use of a polynucleotide or a modified CD8⁺ T cell described herein in the manufacture of a medicament for the treatment of a disease or condition characterized by the expression of MEAF6, SCAMP3, CMV or EBV, in a subject in need thereof. In illustrative embodiments of the invention, the medicament comprises a polynucleotide disclosed herein (e.g., one comprising a TCR disclosed herein). In certain embodiments of the invention, the disease is a cancer expressing a MEAF6 or SCAMP3 polypeptide disclosed herein.

Embodiments of the invention include methods of assessing a patient immune response to a cancer or cancer vaccination (e.g. a prostate cancer or prostate cancer vaccination). Typically, these methods comprise observing the induction or activation of T cells obtained from a patient having a prostate cancer or prostate cancer vaccination, wherein the induction or activation of T cells is observed in response to the T cell's exposure to a polypeptide epitope present on a human MEAF6 or SCAMP3 polypeptide; and an observed induction or activation of T cells provides evidence of patient immune response to cancer or cancer vaccination.

Embodiments of the invention encompass methods of treating a disease or condition characterized by the expression of MEAF6 splice variants or SCAMP3 and/or infection with CMV or EBV. The treatment methodology comprises comprising administering an effective amount of a pharmaceutical composition comprising the modified T cell described herein to a subject in need thereof. The term “subject” is intended to include living organisms in which an immune response can be elicited (e.g., mammals). A “subject” or “patient”, as used therein, may be a human or non-human mammal. Non-human mammals include, for example, livestock and pets, such as ovine, bovine, porcine, canine, feline and murine mammals. Preferably, the subject is human. In typical embodiments of the invention, the human has a cancer expressing a polypeptide epitope recognized by a TCR disclosed herein. In some embodiments of the invention, the cells of the cancer form solid tumors. In illustrative embodiments of the invention, the cancer cells are lung or prostate cancer cells.

A related embodiment of the invention includes a method for prophylaxis and/or therapy of an individual diagnosed with, suspected of having or at risk for developing or recurrence of a cancer, wherein the cancer comprises cancer cells which express polypeptide having an epitope recognized by a TCR. This approach comprises administering to the individual modified human T cells comprising a recombinant polynucleotide encoding a TCR disclosed herein, wherein the T cells are capable of direct recognition of the cancer cells expressing a polypeptide having an epitope recognized by a TCR, and wherein the direct recognition of the cancer cells comprises HLA class I-restricted binding of the TCR to the epitope recognized by the TCR.

With respect to use of the engineered CD8⁺ T cells of the present invention, the method generally comprises administering an effective amount (e.g. by intravenous or intraperitoneal injections) of a composition comprising the CD8⁺ T cells to an individual in need thereof. An appropriate pharmaceutical composition may be adapted for administration by any appropriate route, such as parenteral (including subcutaneous, intramuscular, or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

The technology in this area is fairly developed and a number of methods and materials know in this art can be adapted for use with the invention disclosed herein. Such methods and materials are disclosed, for example in U.S. Patent Publication Nos. 20190247432, 20190119350, 20190002523, 20190002522, 20180371050, 20180057560, 20170029483, 20160024174, and 20150141347, the contents of which are incorporated by reference.

### Experimental

### Results

### Fabrication and Functionalization of Nanovials for T Cells

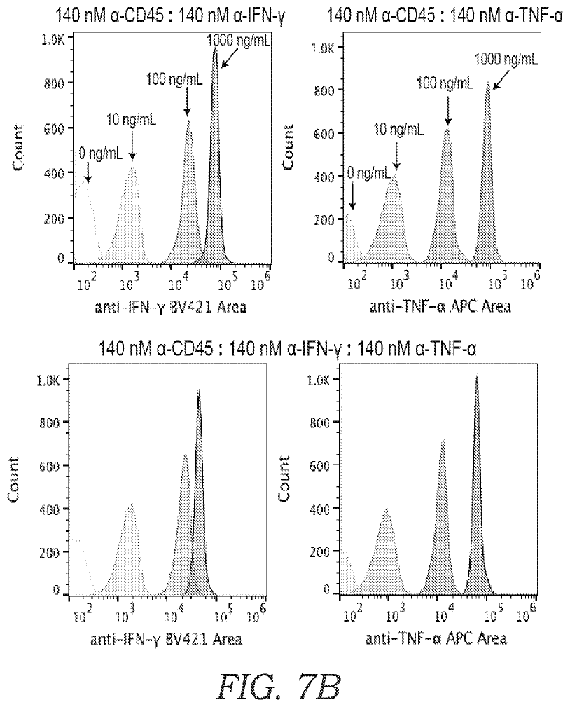

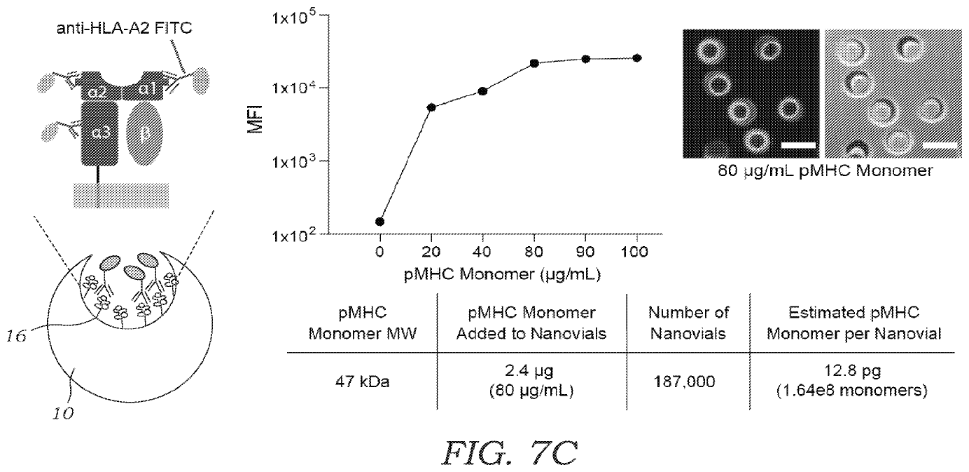

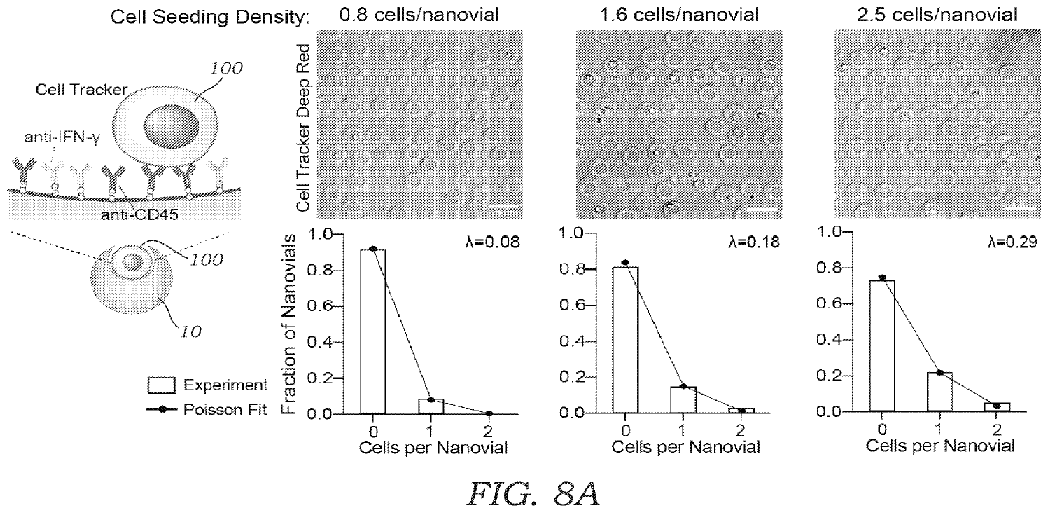

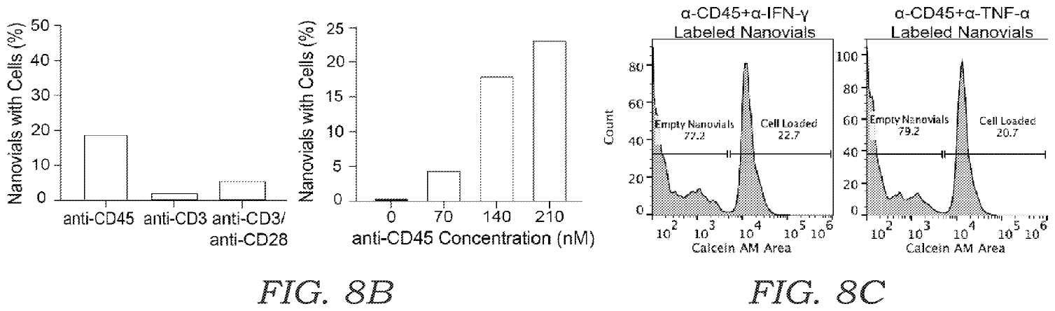

A first aim was to functionalize nanovials 10 to capture T cells 100 and cytokine secretions 22. A microfluidic device, which is illustrated in FIG. 7A, was used that generates uniform water-in-oil emulsions to create millions of monodisperse polyethylene glycol (PEG)-based nanovials 10 with an inner cavity 12 selectively coated with biotinylated gelatin. To accommodate human T cells 100 with diameters of ˜10 μm uniform nanovials 10 were fabricated with an average outer diameter of 35 μm (CV=5.1%) and an average cavity diameter of 21.2 μm (CV=7.2%). By functionalizing the inner cavity 12 with biotin during fabrication one is able to flexibly link multiple biotinylated antibodies (e.g., secretion capture antibodies 18) or peptide-MHC (pMHC) monomers 16 with epitopes of interest through streptavidin-biotin noncovalent interactions (FIGS. 1B and B). For capture and secretion analysis of primary human T cells 100, irrespective of antigen targeting, nanovials 10 were decorated with biotinylated anti-CD45 and cytokine capture antibodies 18 against the following secretions 22: interferon-γ, tumor necrosis factor-α, and interleukin-2 (anti-IFN-γ, anti-TNF-α, anti-IL 2). At least a two order of magnitude dynamic range in detection of recombinant cytokines was observed when anti-CD45 capture antibodies and one, 1:1 (140) nM each) or two cytokine secretion capture antibodies. 1:1:1 (140 nM each) were used during functionalization (FIG. 7B). All of these conditions allowed for T cell loading (FIGS. 8A-8B) as well as signal capture from recombinant cytokines down to 10 ng/ml (FIG. 7B). Cell loading followed Poisson loading statistics with an optimum observed when 1.6 cells per nanovial 10 were seeded (FIG. 8A) with anti-CD45 used to capture cells 100 (FIG. 8B), and was largely independent of the presence of additional cytokine capture antibodies 18 (FIG. 8C). To capture antigen-specific T cells 100, nanovials 10 were decorated with pMHC monomer 16 along with cytokine capture antibodies 18. A linear increase was seen in loaded pMHC monomer signal up to concentrations of 80 μg/mL, resulting in nanovials 10 that could act as artificial antigen presenting cells with high valency of pMHC monomers 16 (FIG. 7C). In order to maintain sites for section capture antibodies 18 targeting secreted cytokines the pMHC concentration was limited to 20 μg/mL for future experiments unless otherwise stated.

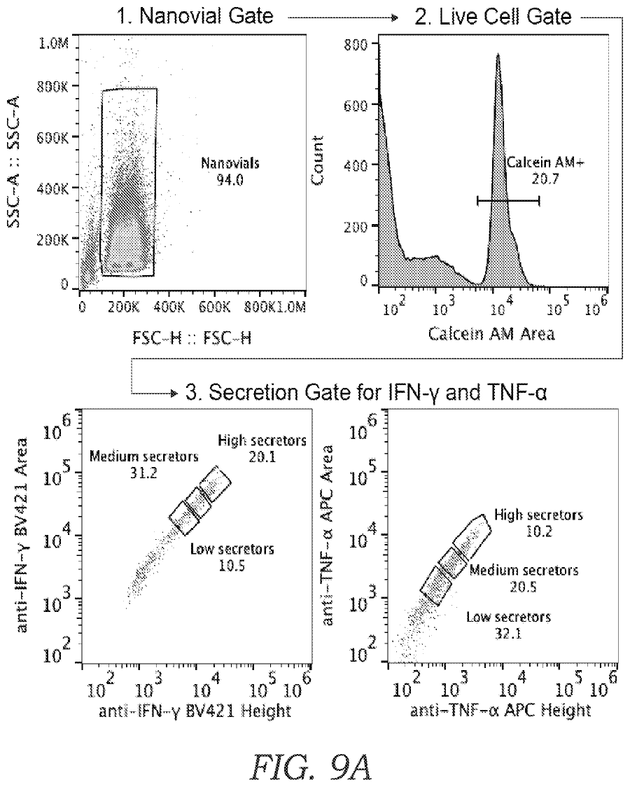

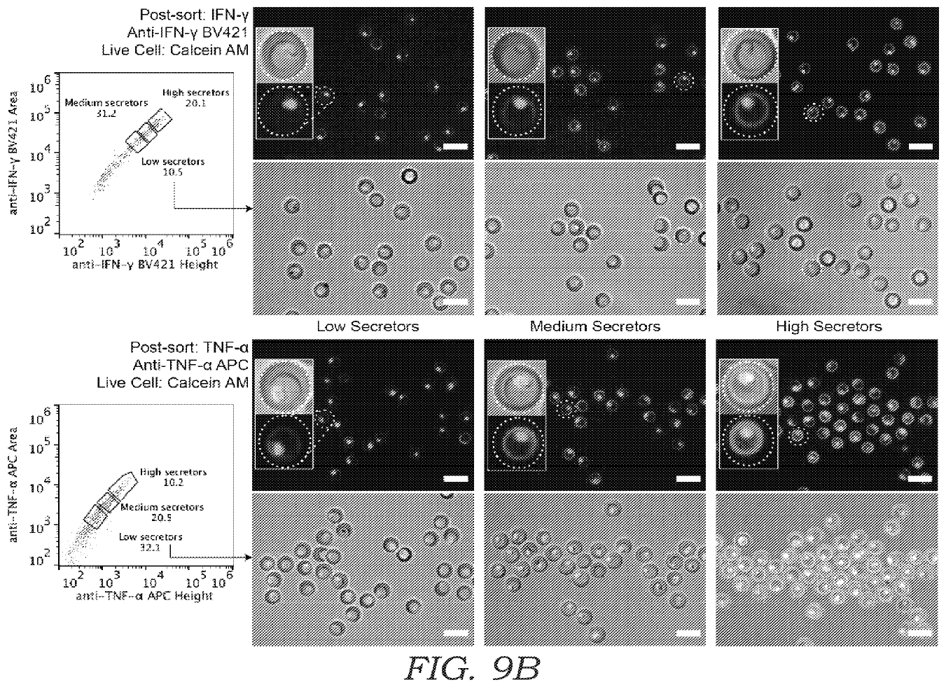

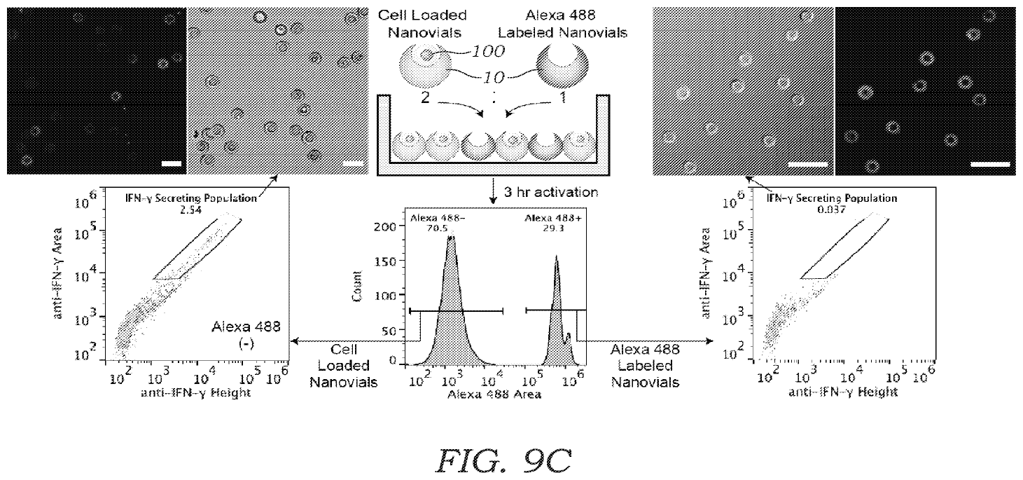

Having validated recombinant cytokine assays and T cell loading on nanovials 10, the processes to accumulate and detect secretions 22 from single T cells 100 bound to nanovials 10 was tested using flow cytometric analysis. Assays were developed for three secreted cytokines 22 (IFN-γ, TNF-α, IL-2) using nanovials 10 coated with the respective individual cytokine capture antibody 18 and anti-CD45. After loading primary human T cells 10 onto nanovials 10. T cells 100 were activated non-specifically with phorbol 12-myristate 13-acetate (PMA) and ionomycin for 3 hours. Following fluorescent staining of captured cytokines 22, a standard cell sorter, the SONY SH800S, was used to sort nanovials 10 with each cytokine secretion signal based on fluorescence peak area and height values. By also gating on a cell viability dye, such as calcein AM, the platform allows for the simultaneous measurement of secretions 22 and viability of individual T cells 100 on nanovials 10, improving the selective sorting of functional T cells 100 (FIG. 9A). Populations of cell-containing nanovials 10 were gated and sorted into low, medium, or high secretors based on the area vs. height plot, recovering viable T cells 100 with different levels of TNF-α and IFN-γ secretion as reflected in fluorescence microscopy images (FIG. 9B). Crosstalk between nanovials 10 was also evaluated by co-culturing cell-loaded nanovials 10 and fluorescently labeled (AlexaFluor 488) nanovials 10 without T cells 100 and found that less than 0.04% of test nanovials 10 without T cells 100 appeared in the positive secretion gate during the 3-hour activation period (FIG. 9C). Presumably, secreted cytokines 22 accumulate at higher concentrations locally but when cytokines diffuse or are advected to neighboring cavities, the concentration is diluted substantially leading to a reduced signal.

Capture of Antigen-Reactive T Cells with pMHC-Labeled Nanovials

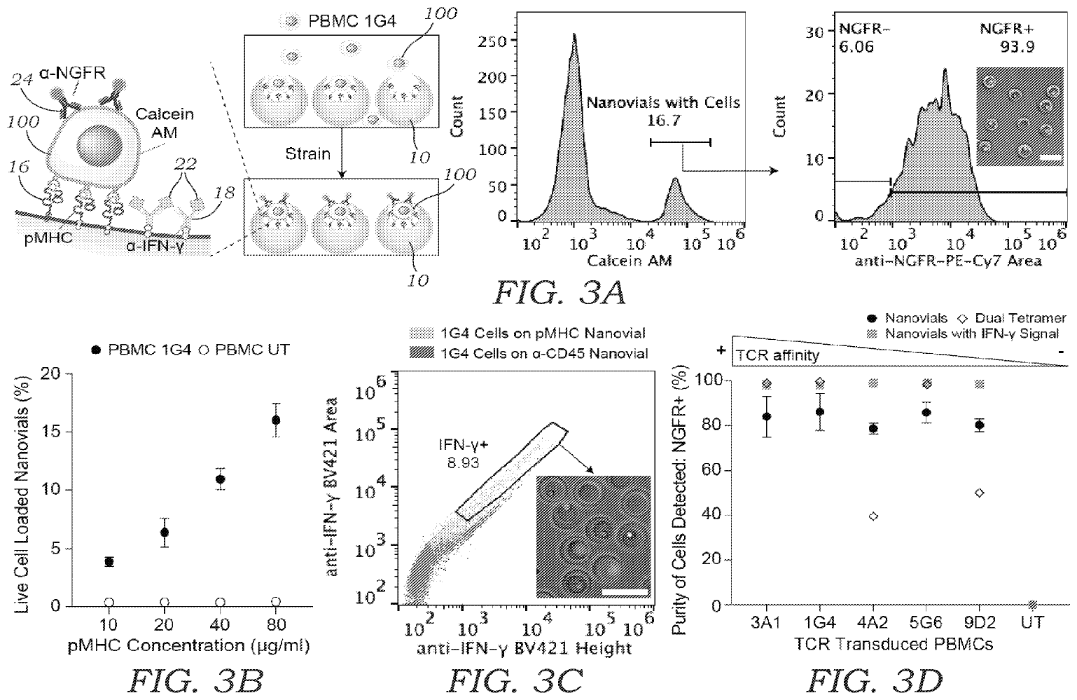

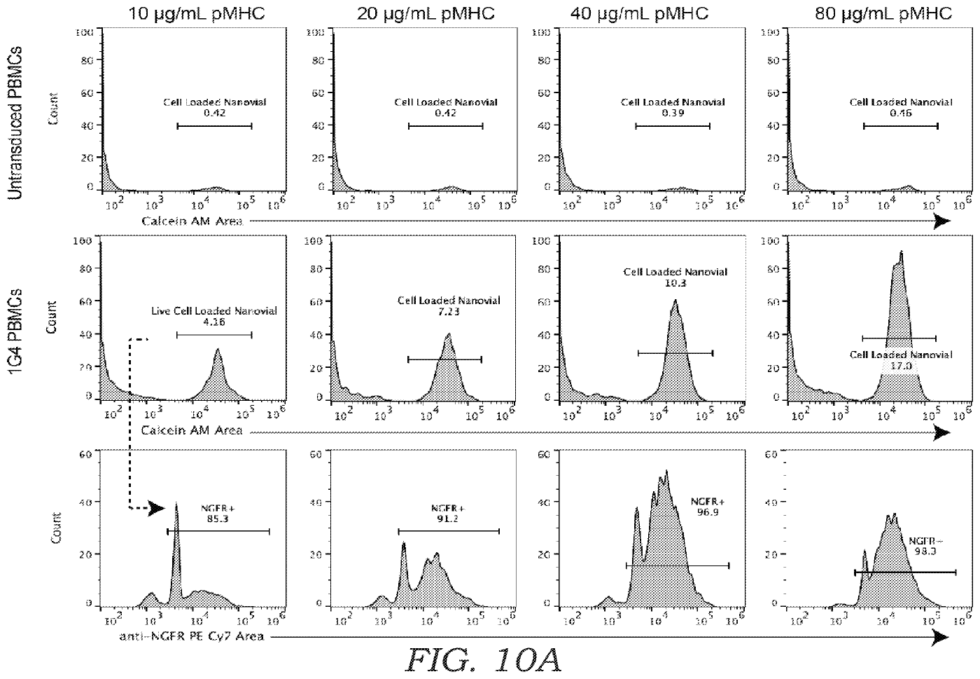

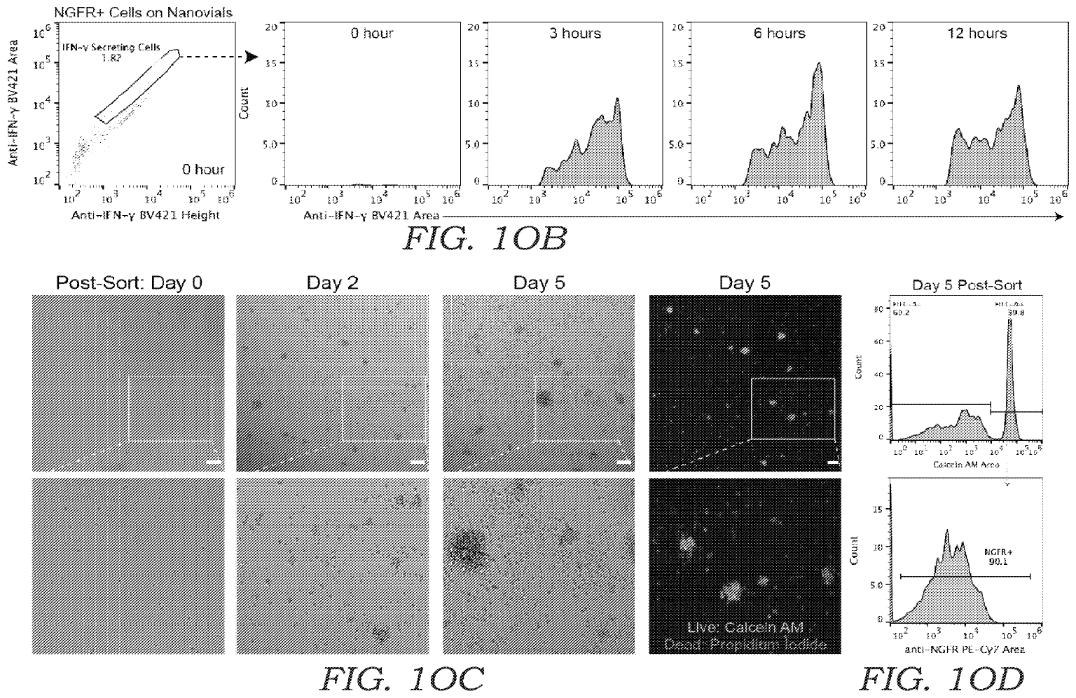

Nanovials 10 coated with pMHC 16 and cytokine capture antibodies 18 could be used for antigen-specific capture. TCR-specific activation, and detection of secreting cytokines. Transitioning from using anti-CD45, pMHC-functionalized nanovials 10 were applied for the selection of antigen-reactive T cells 100. The specificity of nanovials 10 in selectively binding antigen-specific T cells 10 was analyzed using human peripheral blood mononuclear cells (PBMCs) transduced with 1G4 TCR targeting NY-ESO-1, a clinically studied cancer-specific antigen (FIG. 3A). Truncated nerve growth factor receptor (NGFR) was used as the co-transduction marker for the presence of 1G4 TCR. PBMCs transduced with and expressing 1G4 bound specifically to nanovials 10 labeled with pMHC monomer 16 containing HLA-A*02:01 restricted NY-ESO-1 C9V peptide SEQ ID NO: 1: (SLLMWITQV) (20 μg/ml) (FIG. 2A). Live cells occupied ˜17% of nanovials, and 93.9% had NGFR expression. To further clarify if the interaction was specific, the NY-ESO-1 pMHC concentration was increased used to functionalize nanovials 10. A corresponding increase in the amount of antigen-specific T cells 100 loaded was observed in a dose-dependent manner, while the binding of untransduced PBMCs was low and independent of pMHC concentration (FIG. 3B. FIG. 10A).

To investigate whether pMHCs 16 on nanovials 10 can specifically trigger activation and secretion from engaged antigen-specific T cells 100. 1G4-transduced cells were each loaded onto nanovials 10 labeled with pMHC monomers 16 or anti-CD45 antibodies. T Cells 100 loaded on control anti-CD45 labeled nanovials 10 (red dots) had low levels of IFN-γ signal, which was mostly associated with non-specific staining of cells, while cells on NY-ESO-1 pMHC-labeled nanovials (cyan dots) clearly secreted IFN-γ as early as 3 hours after loading (FIG. 10B), yielding 1-2 orders of magnitude higher fluorescence intensity observed at larger area:height ratios (FIG. 3C). Surprisingly, the induction of T Cell 100 secretion of cytokines 22 was achieved on nanovials 10 without the addition of signal 2 (such as CD28 binding to CD80 on nanovials 10) which is present in a natural immune synapse.

For some therapeutic workflows enrichment and regrowth of rare antigen-reactive populations are required. To assess proliferation of antigen-specific T cells 100 after isolation, 1G4-transduced PBMCs enriched on NY-ESO-1 pMHC-coated nanovials 10 were first sorted and detached using collagenase D. T Cells 100 expanded in culture over 5 days, with 90% of the expanded population expressing the 1G4 TCR, showing enrichment and continued growth of the antigen-specific T cell population (FIG. 10C).

T Cells with Low Affinity TCRs are Isolated Effectively by pMHC-Coated Nanovials

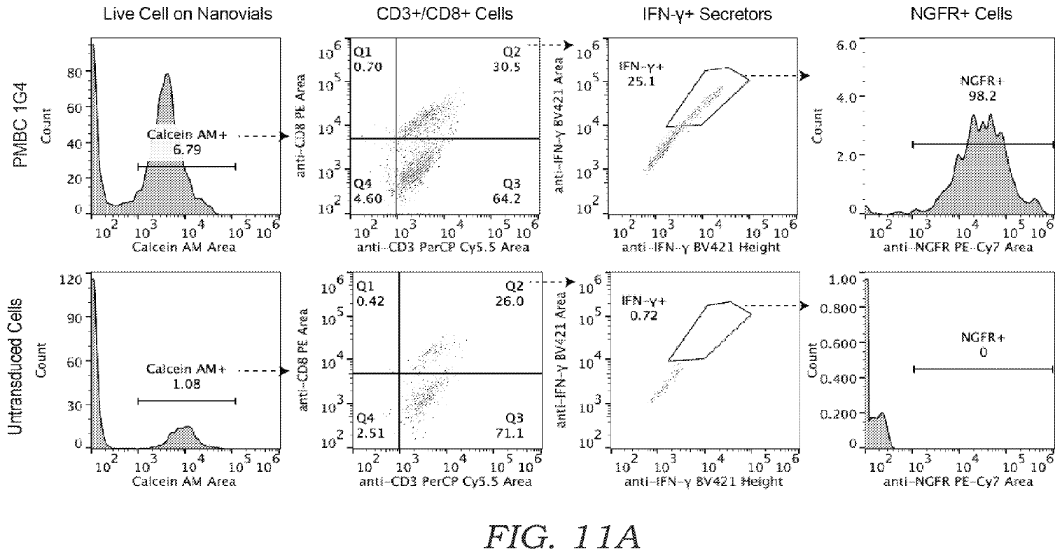

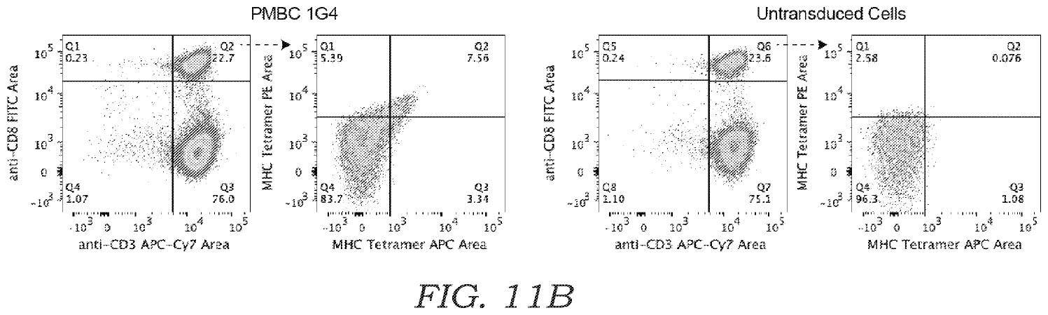

Since the 1G4 TCR has high affinity to NY-ESO-1 pMHC, it was questioned whether increased avidity of pMHCs 16 coating the nanovial cavity 12 would prove advantageous in recovering TCRs with various affinities. Human PBMCs were transduced with five previously identified TCRs (3A1, 1G4, 4A2, 5G6, 9D2) targeting the same HLA-A*02:01 restricted NY-ESO-1 C9V peptide. The relative affinities of these TCRs were assessed using fluorescent MHC dextramer binding. The purity of antigen-specific cells was compared by nanovial capture, nanovial capture gated on IFN-γ secretion, and dual-color tetramer staining (FIG. 3D). The purity of detected cells 100 was defined as the fraction of NGFR+ cells from CD3+/CD8+ cells on nanovials 10 with or without IFN-γ secretion signal, or fraction of NGFR+ cells 100 from dual-tetramer⁺ cells 100 (FIGS. 11A-11B). The purity of isolated cells 100 on nanovials 10 approaches 100% for NGFR+ cells 100 when also considering IFN-γ signal (FIG. 3D). pMHC-labeled nanovials 10 also recovered more antigen-specific T cells 100 than dual-color tetramer, especially when cells 100 possessed low-affinity TCRs (4A2, 5G6, 9D2) as represented by the total NGFR+ cells in Table 1.

TABLE 1

| Tetramers | | Nanovials | |
|---|---|---|---|
| TCR | | | CD3+CD8+ |
| Transduced | | | IFN-γ+ |
| PBMCs | CD3+CD8+Tetramer+NGFR+ | CD3+CD8+NGFR+ | NGFR+ |
| 3A1 | 4017 | 1874 | 653 |
| 1G4 | 1797 | 1775 | 571 |
| 4A2 | 15 | 1996 | 447 |
| 5G6 | 98 | 2222 | 888 |
| 9D2 | 9 | 953 | 362 |
| UT | 0 | 0 | 0 |

### Recovery of Functional Viral-Epitope-Specific TCRs Using Nanovials

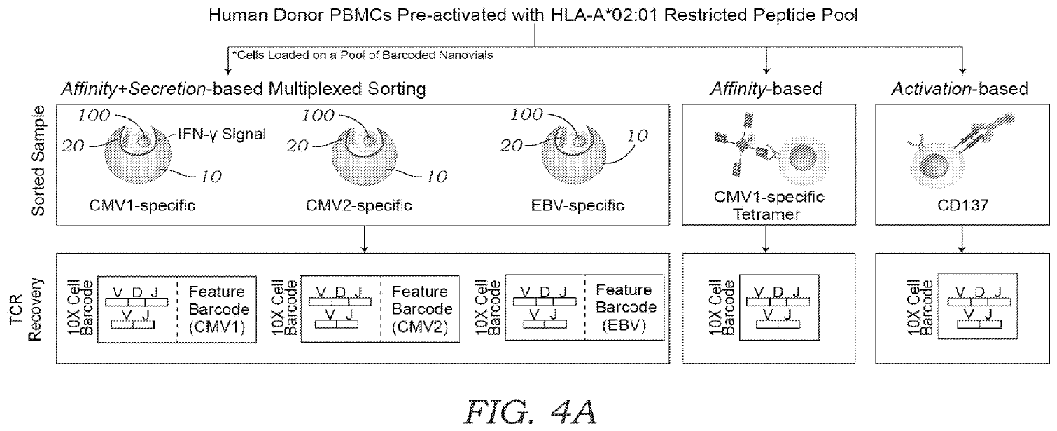

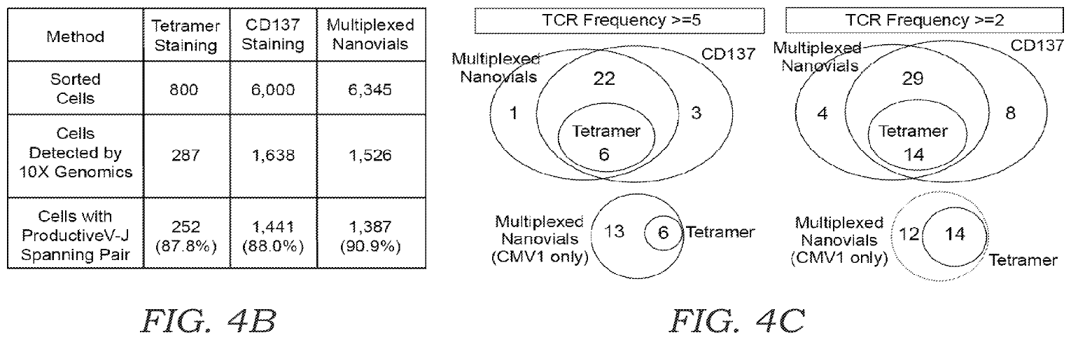

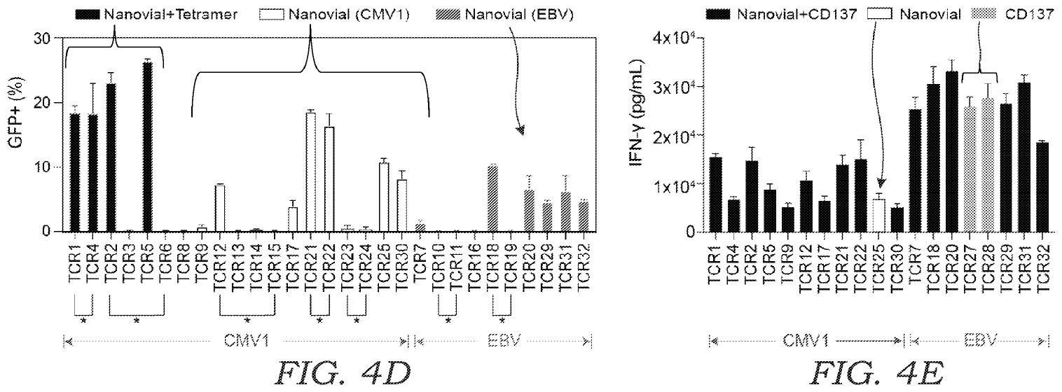

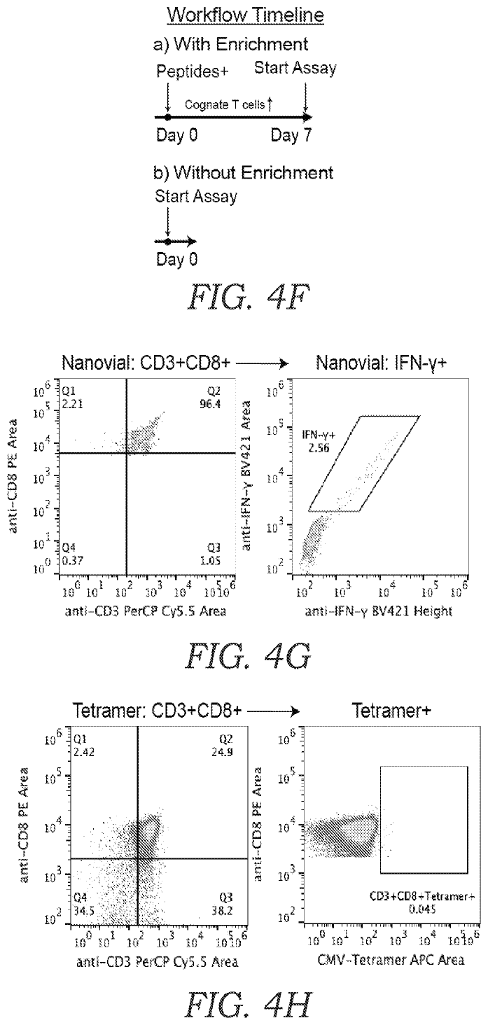

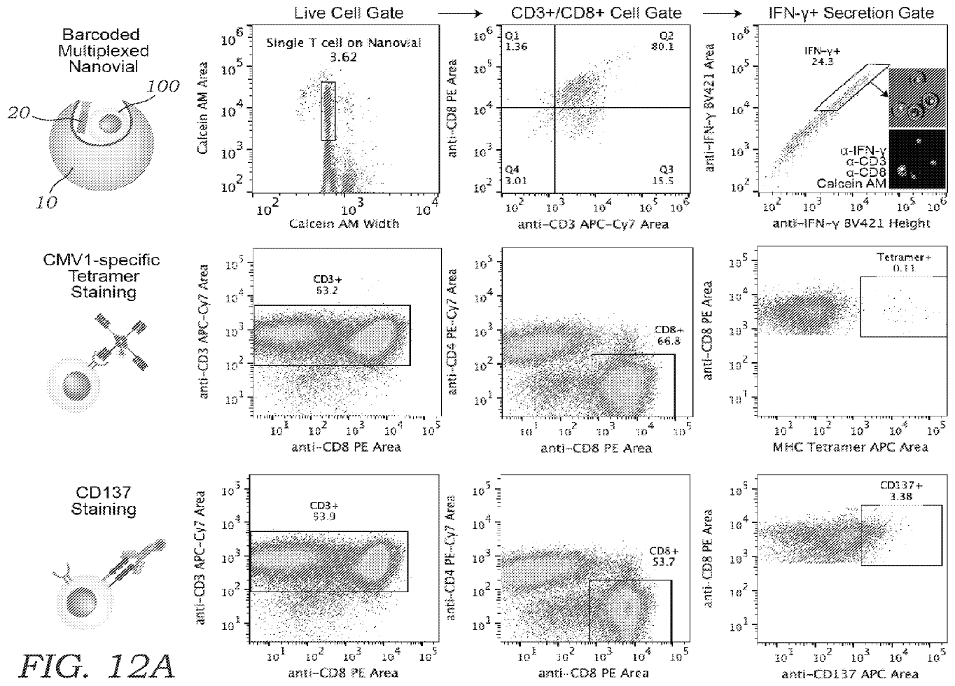

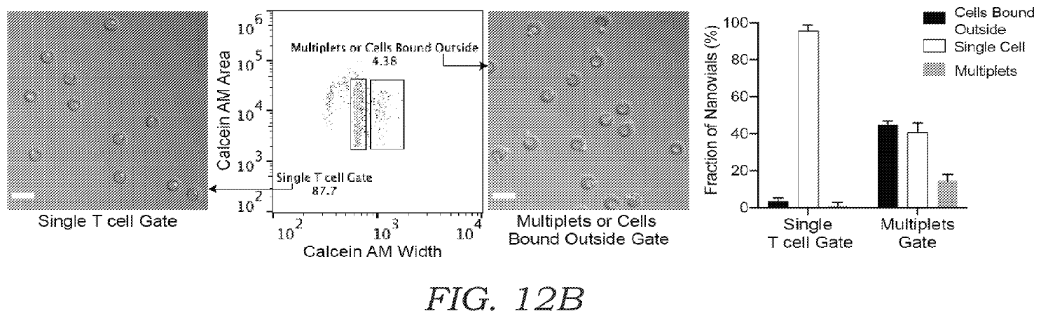

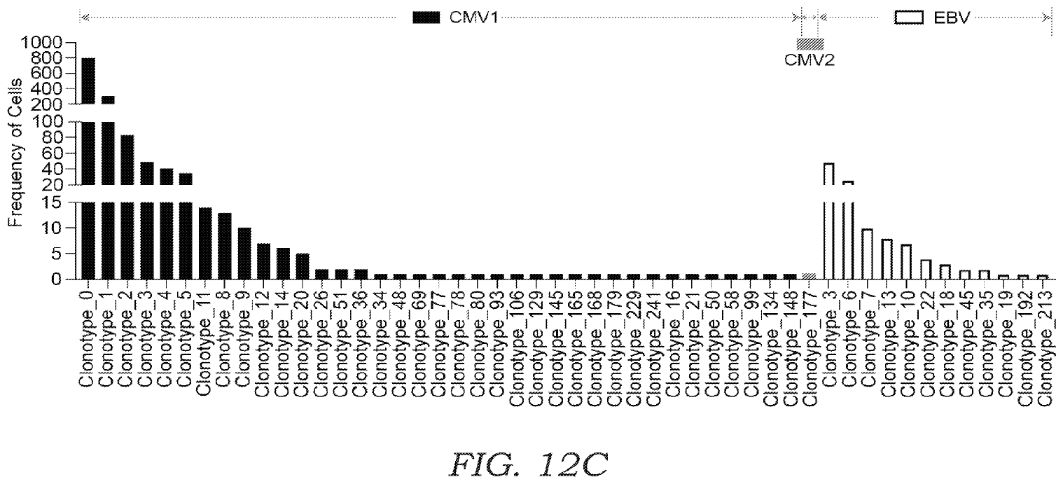

Following successful isolation of rare antigen-specific T cells 100 with TCRs of varying affinities in model systems it was hypothesized that sorting based on a combination of binding and cytokine secretion using nanovials 10 would increase the functional hit rate of a diverse repertoire of TCRs specific to common viral epitopes (or other epitopes). Healthy donor PBMCs pre-activated with a pool of previously reported HLA-A*02:01 restricted peptides from cytomegalovirus (CMV) and Epstein Barr virus (EBV) targeting CMV pp65 (CMV1) SEQ ID NO 2: (NLVPMVATV). CMV IE-1 (CMV2) SEQ ID NO 3: (VLEETSVML), and EBV BMLF1 (EBV) SEQ ID NO 4: (GLCTLVAML) were isolated using three different methods: secretion based sorting using nanovials 10, sorting using a CMV pp65-specific tetramer, or activation-based sorting using CD137 as the surface marker (FIG. 4A). The detection of antigen-specific T cells 100 was multiplexed by loading cells 100 onto a pool of barcoded nanovials 10 labeled with three HLA-A*02:01 restricted pMHCs 16 targeting each antigen (CMV1, CMV2, EBV) and a corresponding oligonucleotide barcode 20. Approximately 6000 CD3⁺CD8⁺ cells 100 on nanovials 10 associated with IFN-γ secretion signal and 800 CMV1-specific pMHC tetramer+ cells were identified and sorted from the entire sample (FIGS. 12A-12C. FIG. 4B). To have an equivalent starting cell number for single-cell sequencing. 6000 cells were sorted based on gating for above background levels of the CD137 activation marker (FIGS. 12A-12C).

TCRs were recovered from sorted cells using the 10× Genomics Chromium platform (FIG. 4B). Notably, the cells 100 on nanovials 10 were introduced directly into the system to maintain the connection between a nanovial 10 with a feature oligonucleotide barcode 20 tag and the attached T cell 100. Nanovials 10 did not interfere with the gene sequence recovery resulting in the highest fraction of T cells 100 with a productive V-J spanning pair (90.9%) compared to tetramer (87.8%) and CD137 samples (88%) (FIG. 3B). A list of high-frequency TCR clonotypes (frequency≥5) detected by the three methods was compiled. Since a few clonotypes contained multiple alpha or beta chains, these were recombined into separate TCR sequences with each permutation of alpha and beta chains. In total, 32 unique TCR pairs were retrieved with frequency≥5: Six (6) TCRs overlapped among the three methods, twenty-eight (28) overlapped between the nanovial 10 and CD137 approaches, and one (1) unique TCR was detected with nanovials 10 (FIG. 4C). A larger number of unique TCR sequences were detected with a less stringent cutoff of frequency≥2, where the overlapping number of TCRs between nanovial and CD137 techniques increased from 28 to 43, suggesting additional rarer TCRs were also discovered.

### Barcoded Nanovials Reveal Epitope Information During T Cell Isolation

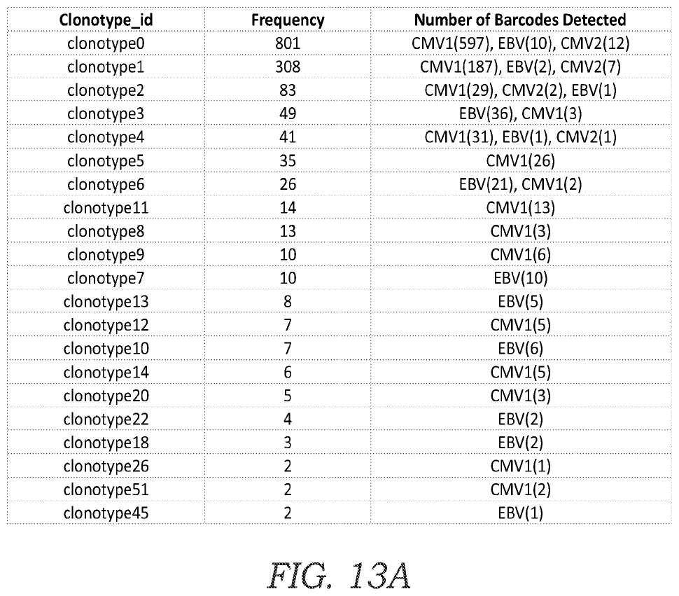

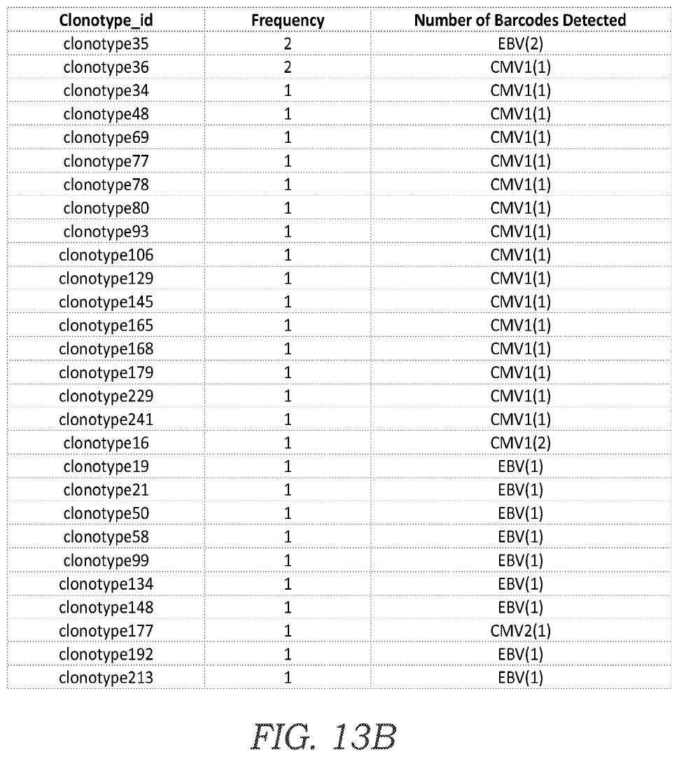

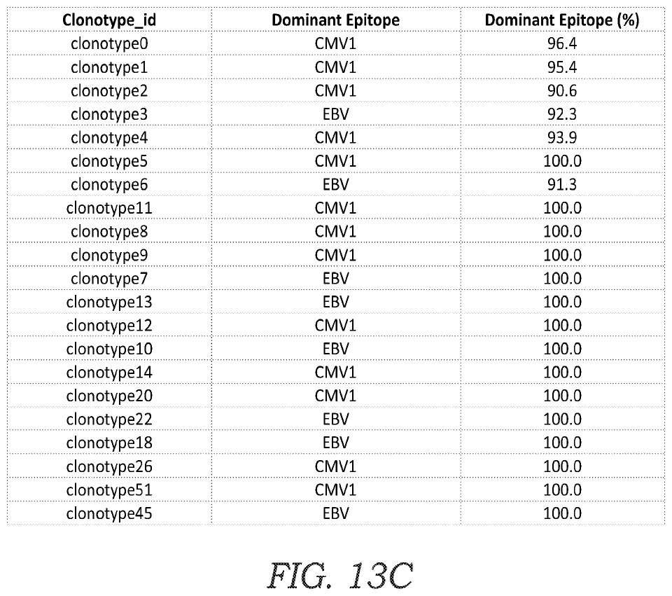

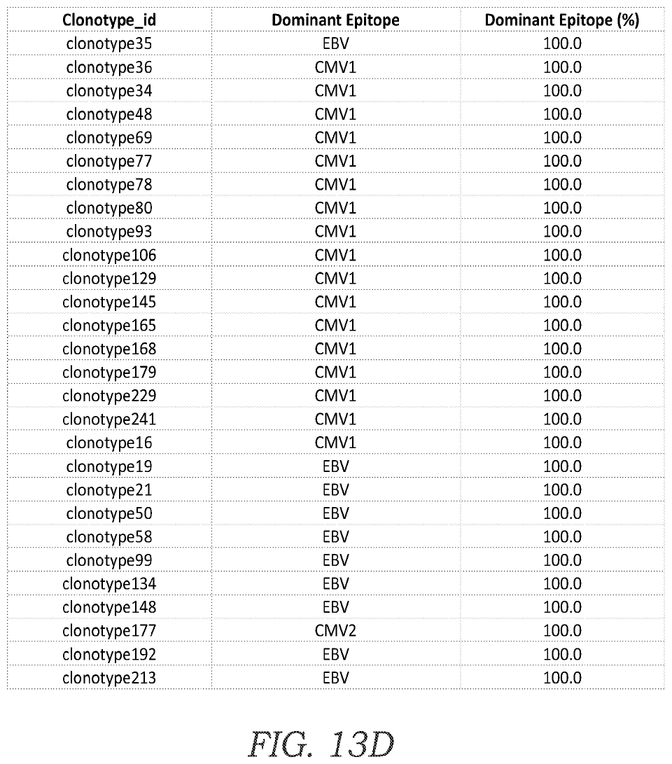

Unlike workflows using CD137, which require laborious deconvolution to uncover the target epitopes from a peptide pool that match specific TCR sequences, pMHC-barcoded and multiplexed nanovials 10 reveal epitope information during cognate T cell isolation. Using the 10× Chromium system. TCR sequence information of each T cell 100 was linked to the nanovial pMHC feature barcode (using the oligonucleotide barcode 20), resulting in the recovery of each TCR with matching target epitope information. A >90% frequency of the pMHC barcode 20 identified the dominant epitope for each TCR (FIGS. 13A-13D). The distribution of clonotype frequency with the corresponding epitope is represented in FIG. 12C for the nanovial workflow. 32 CMV1 (CMV pp65), 1 CMV2 (CMV IE1) and 12 EBV-reactive clonotypes were detected and 96.4% of sequenced cells 100 with productive V(D)J spanning pairs had matching epitope information.

To understand the antigen-specific reactivity of 32 unique TCR sequences from 26 clonotypes (one clonotype may contain multiple alpha and beta chains) retrieved by the three methods (nanovial, tetramer, CD137) with a frequency≥5, candidates were re-expressed via electroporation into Jurkat-NFAT-GFP cells, in which GFP expression can be induced upon TCR recognition. Murine constant regions were used for both TCR alpha and beta chains to prevent mispairing with endogenous TCRs. Engineered Jurkat cells were then co-cultured with K562 cells expressing HLA-A*02:01 (K562-A2) as antigen-presenting cells along with exogenously added peptides. Activation of the Jurkat cells was determined by flow cytometry, gating on % of the CD8⁺/murineTCRβ⁺ population with GFP signal above background. From the smaller pool of CMV1-specific TCRs, the nanovial workflow yielded 6 more reactive TCRs compared to CMV1 pMHC tetramer labeling (FIG. 4D). Out of the 29 possible TCR combinations identified with nanovials 10, seventeen (17) were found to be reactive upon re-expression (11 for CMV1 and 6 for EBV) (FIG. 4D). Notably, some of these TCRs were from cells 100 in which sequencing yielded multiple alpha and/or beta chains (denoted with connecting line and an asterisk. FIG. 4D). When collapsing these related TCR clonotypes to individual cell clonotypes, it was found that 78% of clonotypes recovered using nanovials 10 had at least one TCR permutation with antigen-specific reactivity and the calling of epitope information was accurate for those reactive TCRs. The few non-reactive TCRs were tested with the other peptides and found to be unreactive.

To investigate how functional IFN-γ secretion-based selection on nanovials 10 correlated to secretory function elicited by the recovered TCR sequences in T cells 100, nineteen (19) reactive TCRs identified in the Jurkat-NFAT-GFP assay were transduced into human PBMCs and IFN-γ secretion was measured following exposure to antigen presenting cells (APCs) with exogenously added cognate peptides. It was found that T cells 100 transduced with all nineteen (19) reactive TCRs tested were able to specifically produce secreted IFN-γ (>5000 μg/mL) when stimulated by APCs presenting exogenous peptides (FIG. 4E). Levels of secreted of IFN-γ in PBMCs were not directly correlated to GFP activation signals when tested in Jurkat-NFAT-GFP (R²=0.10). Nanovial 10 and CD137 approaches were both able to recover TCRs with a range of different potencies, but only nanovials 10 provided matched epitope information.

### Direct Enrichment of Antigen-Specific T Cells on Nanovials and Comparison to Tetramers

A pre-activation expansion step of PBMCs was used to enrich reactive T cells 100 in one experiment (FIG. 4F), requiring an additional 7 days of culture. Nanovials 10 were also used to directly enrich and activate T cells 100 from freshly thawed PBMCs. PBMCs were directly loaded onto nanovials 10 or performed tetramer staining, both using pMHC CMV1 (CMV pp65, SEQ ID NO. 2 (NLVPMVATV). 10⁷PBMCs were used in each method without pre-expansion, which reduces a week-long experiment to a single day (FIG. 4F). Antigen-specific T cells 100 that bound to CMV1 on nanovials 10 and secreted IFN-γ, or bound to CMV1 on tetramers were gated and recovered by the two methods (FIG. 4G, 4H). pMHC-labeled nanovials 10 were able to recover ˜13,000 CD3+CD8+ cells with a clear fraction of bound cells (398) secreting IFN-γ (Table 2).). Notably, secretion was observed even though these PBMCs were not pre-activated and no signal 2 receptors were present on the nanovials 10. For the same sample of PBMCs, using dual-color tetramers yielded 163 CD3+CD8+ cells (Table 2).

TABLE 2

| Nanovials | | Tetramers | |
|---|---|---|---|
| Starting Cell Number | 10⁷ | Starting Cell Number | 10⁷ |
| CalceinAM + | 13111 | Tetramer+/CD3+CD8+ | 163 |
| CD3+CD8+ Cells | | Cells | |
| on Nanovials | | | |
| CD3+CD8+IFN-γ+ | 398 | | |
| Cells on Nanovials | | | |

### Detection of Antigen-Specific T Cells Based on Granzyme B Secretion Using Nanovials

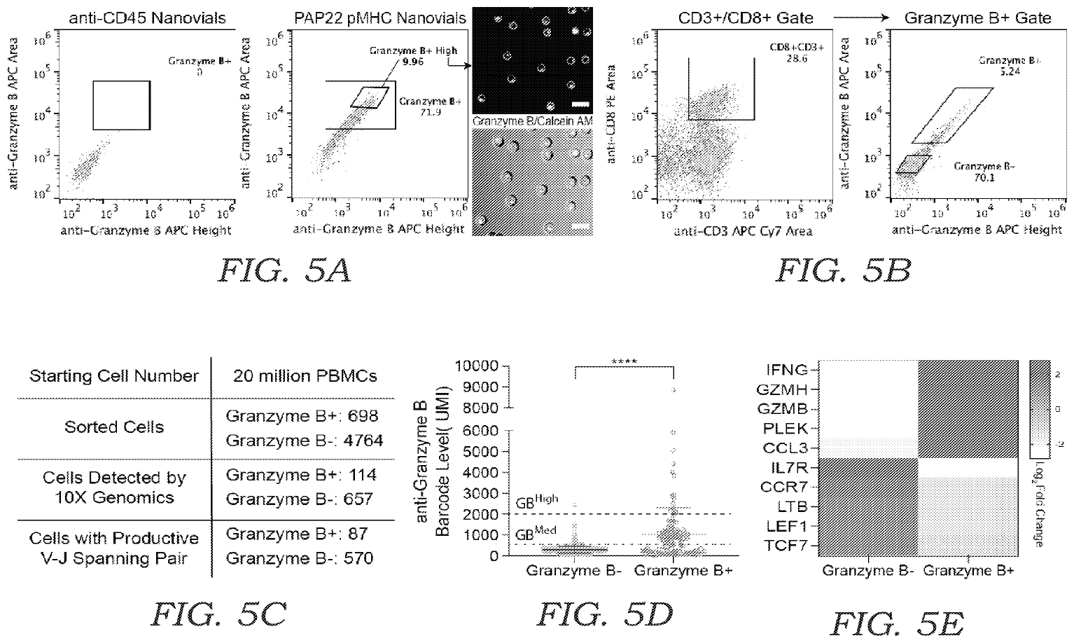

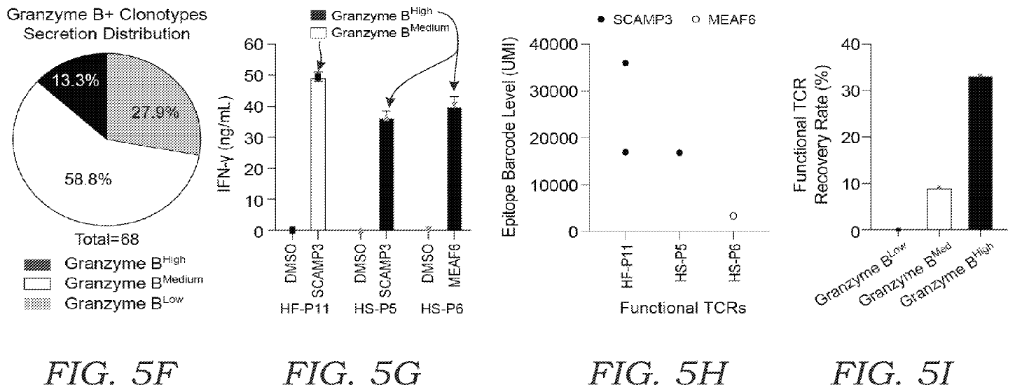

IFN-γ signaling is primarily associated with activated T cells 100 and cell-mediated immune responses. As more direct evidence for cytotoxicity of antigen-specific T cells 100, the nanovial assay was further expanded for the isolation of T cells 100 based on granzyme B as the secretion 22, which remains challenging by currently available techniques. A previously identified TCR (TCR156) targeting a defined epitope (PAP22) of prostatic acid phosphatase (PAP), a prostate tissue antigen, was used to validate this approach. This low-affinity TCR shows antigen-specific recognition but weak tetramer signals in reconstruction experiments. In the context of HLA-A*02:01, TCR156 transduced PBMCs were loaded onto anti-CD45-labeled or PAP22 pMHC-labeled nanovials 10 and granzyme B secretion was analyzed after 3 hours of activation. Strong granzyme B secretion was only observed from the cells 100 that bound to pMHC-labeled nanovials 10, showing antigen-specific activation (FIG. 5A). By sorting the top 10% of granzyme B secreting cells 100, viability and intense secretion signal was confirmed on the nanovial cavity 12 by fluorescence microscopy (FIG. 5A).

### Discovery of Rare Functional TCRs Targeting Prostate Cancer Epitopes

The nanovial platform was then used for the recovery of rare functional TCRs targeting PAP and cancer-enhanced splicing peptides from human donor PBMCs. Previous studies indicate the frequency of finding cognate TCRs against those epitopes is extremely low. In this experiment, the number of nanovial types was expanded to ten (10) different HLA-A*02:01 restricted pMHC-labeled barcoded sets: PAP14 SEQ ID NO 125: (ILLWQPIPV), PAP21 SEQ ID NO 126: (LLLARAASLSL), PAP22 SEQ ID NO 127: (TLMSAMTNL), PAP23 SEQ ID NO 128: (LLFFWLDRSVLA), CTNND1 SEQ ID NO 129: (MQDEGQESL), CLASP1 SEQ ID NO 130: (SLDGTTTKA), MEAF6 SEQ ID NO 131: (SGMFDYDFEYV), PXDN SEQ ID NO 132: (HLFDSVFRFL), SCAMP3 SEQ ID NO 133: (STMYYLWML), and TCF12 SEQ ID NO 134: (SLHSLKNRV), all of which have been previously used for TCR discovery. In order to increase the confidence in re-expressing potential rare TCRs with low frequency of recovery, a new capability was introduced into the nanovial assay where cell secretion of granzyme B is linked to the TCR sequence information by adding a barcoded secondary antibody 26 which was an oligo-nucleotide barcoded antibody that reports out the level of granzyme B secretion. In this case an oligo-anti-APC antibody 26 targeting anti-granzyme B-APC was added. The goal was to be able to rank TCR sequences by the amount of granzyme B associated with T cells 100 expressing that TCR. Starting with 20 million donor PBMCs from one healthy donor, live+CD3+CD8+ cells that bound to nanovials 10 and had granzyme B signal above the gate (granzyme B+, 698 cells) were sorted (FIG. 5B). A subset of live+CD3+CD8+ cells on nanovials 10 below the granzyme B secretion threshold (granzyme B−, 4764 cells) were also sorted as a negative control (FIG. 5B). Using the 10× Genomics platform, libraries were constructed for V(D)J sequences, the 1^{st} feature barcode (i.e., oligonucleotide barcode 20) encoding the specific pMHC molecule (of 10 types) on nanovial 10, the 2^{nd} feature barcode (i.e., barcoded secondary antibody 26) encoding granzyme B secretion level, and gene expression. In total. 87 cells 100 were sequenced and recovered with a productive V-J spanning pair from the Granzyme B+ population and 570 cells 100 from the Granzyme B− population (FIG. 5C).

Using the oligo-barcoded detection antibodies 26 targeting the granzyme B signal, the secretion level for each TCR clonotype was determined (FIG. 5D). As expected, the average secretion barcode level of Granzyme B+ clonotypes (Mean=1022, SD=1286) was significantly higher (p<0.0001) than the Granzyme B− clonotypes (Mean=288, SD=149), representing that the oligo-barcoding process accurately reflects the fluorescence gates (FIG. 5D). Notably, the three most differentially expressed up-regulated genes among the Granzyme B+ population as compared to the Granzyme B− population were IFN-γ, granzyme H, and granzyme B, which supports the idea that granzyme B and IFN-γ act as crucial effectors for inducing cytotoxic activity (FIG. 5E).

Based on the distribution of granzyme B secretion barcode levels, each clonotype was categorized into three different classes: Granzyme B^{High} (barcode level≥2000). Granzyme B^{Medium} (2000>barcode level≥500. Granzyme Blow (barcode level<500) (FIG. 5D). Among the 68 Granzyme B+ clonotypes, 40 clonotypes fell under Granzyme B^{Medium} (58.8%), 9 clonotypes under Granzyme B^{High} (13.3%) and 19 clonotypes under Granzyme B^{Low} (27.9%) (FIG. 5F).

### Functional Validation of TCRs Recovered Based on Secretion Barcode Levels

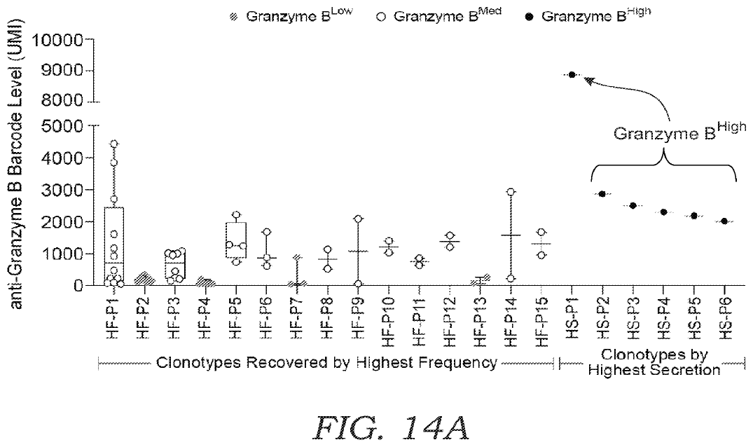

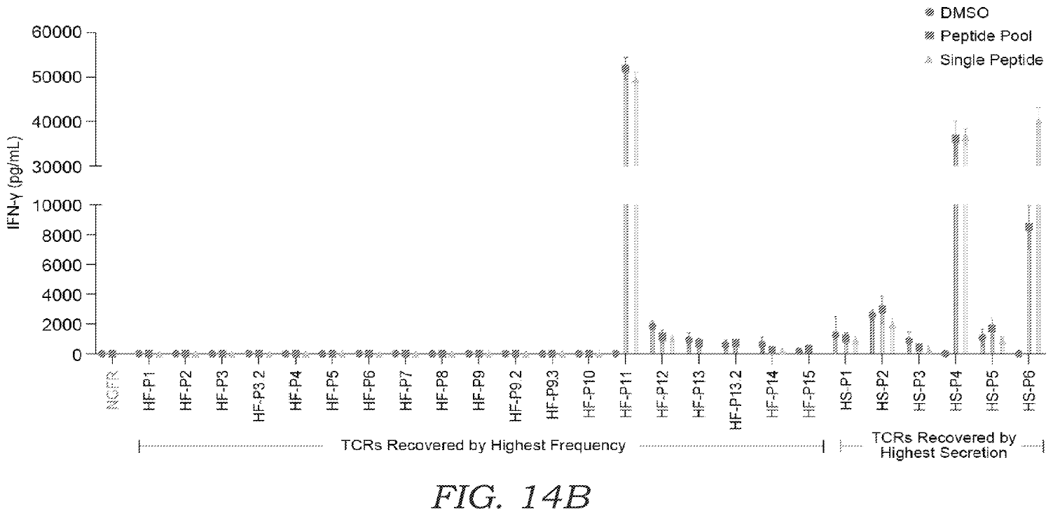

T cells 100 with the highest levels of granzyme B yielded the most potent TCRs with highest reactivity. The top 6 clonotypes from the Granzyme B+ population were selected and ranked, expressing granzyme B secretion barcode levels above 2000 with productive TCR alpha and beta chains (HS-P1 to HS-P6) (FIG. 14A). In comparison, the 15 most frequent clonotypes were tested, following common practice for identifying potent TCRs (HF-P1 to HF-P15). Interestingly, no clonotypes in the high frequency list overlapped with the Granzyme B^{high} clonotypes (FIG. 14A).

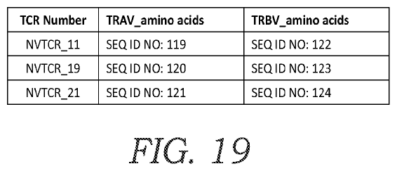

To evaluate the functionality of recovered clonotypes in these separate lists, each candidate was re-expressed in human PBMCs and IFN-γ secretion was measured following exposure to antigen presenting cells (APCs) with exogenously added cognate peptides (peptide pool or a single-peptide noted from the nanovial barcode encoding the specific pMHC molecule). A few clonotypes containing αβ chain permutation were recombined into a separate TCR sequence with each permutation of αβ chains. In total, 25 unique TCRs were validated: 19 TCRs recovered based on high frequency and 6 TCRs recovered based on high secretion (FIG. 14B). From PBMCs of one healthy donor, 2 functional TCRs were discovered from the Granzyme B^{High} and 1 from Granzyme B^{Medium} TCRs that secreted IFN-γ upon exposure to APCs (FIG. 5G; see TCRs NVTCR_11, NVTCR_19, or NVTCR_21 in FIG. 19). For those 3 functional TCRs, nanovials 10 also provided accurate epitope information (FIG. 5H). The recovery rate of functional TCRs from Granzyme B^{high} was the highest (33%) as compared to Granzyme B^{Medium} (6.7%) and Granzyme B^{Low} (0%) TCRs, suggesting that functional (secretion) information captured by nanovials 10 improves the detection rate of rare and potent TCRs (FIG. 5I).

### Multiplexed Secretion-Based Profiling

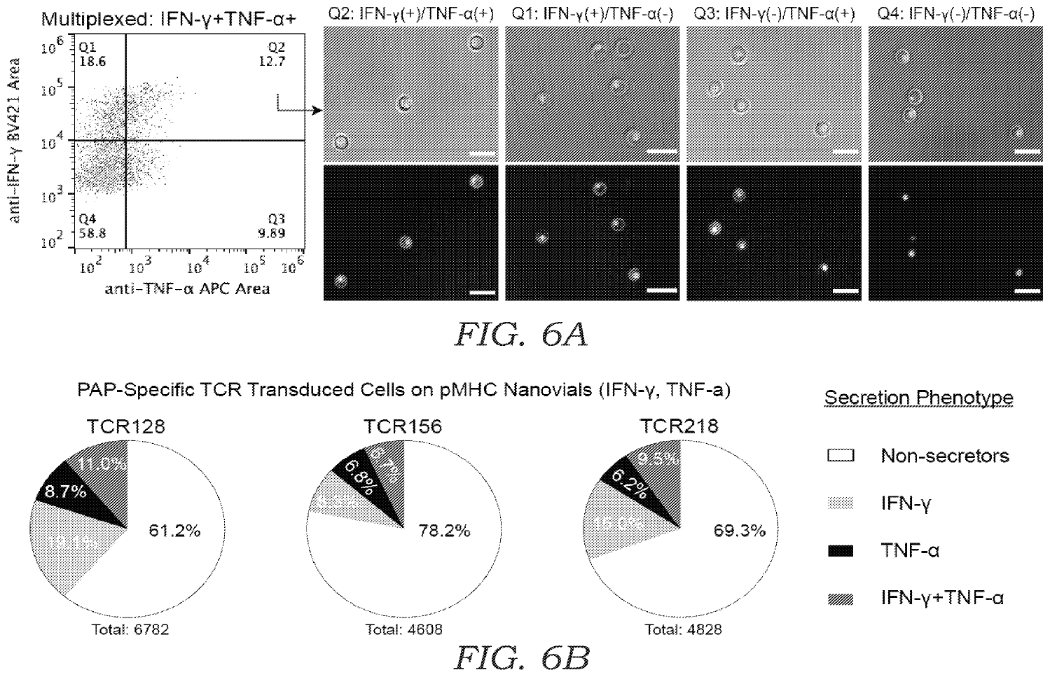

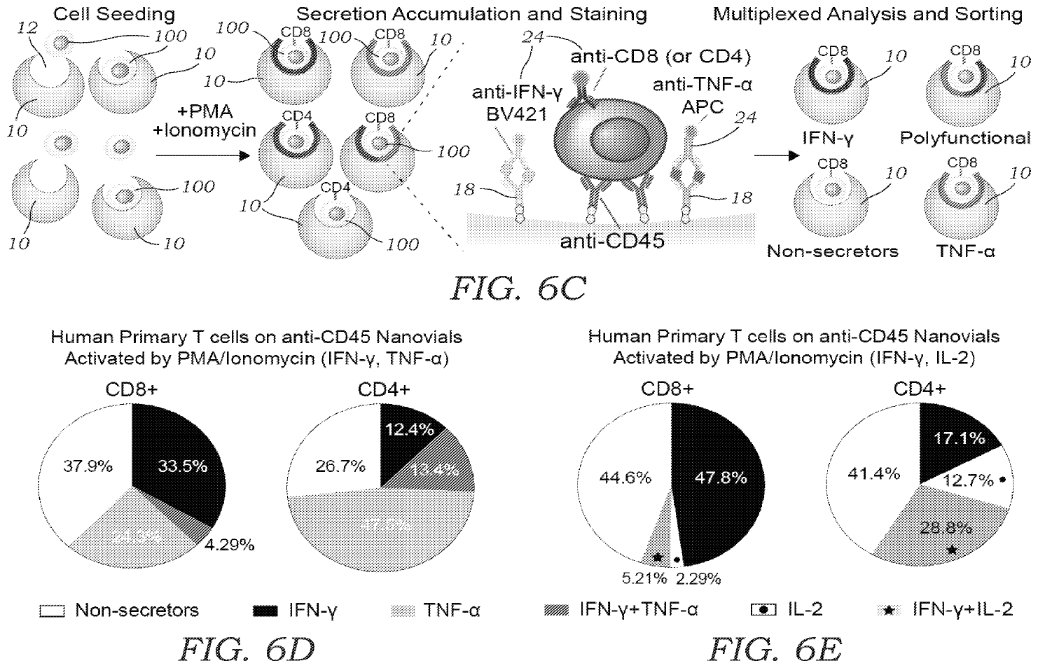

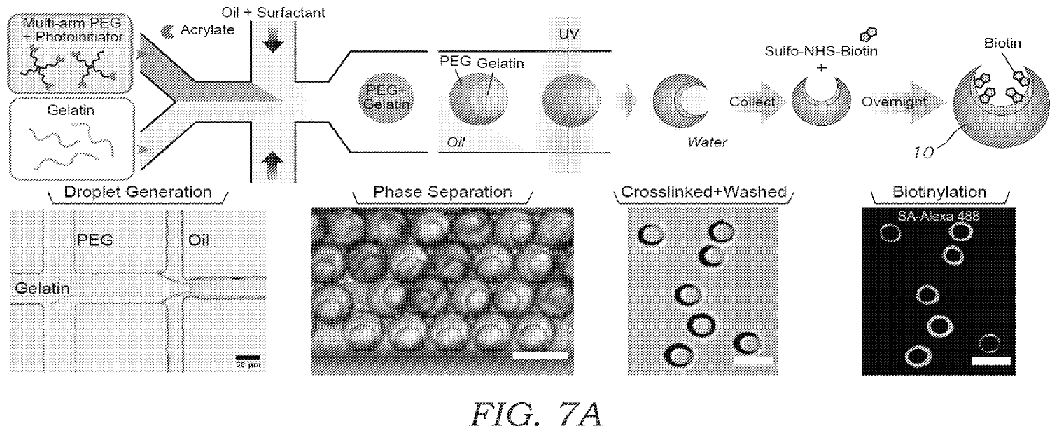

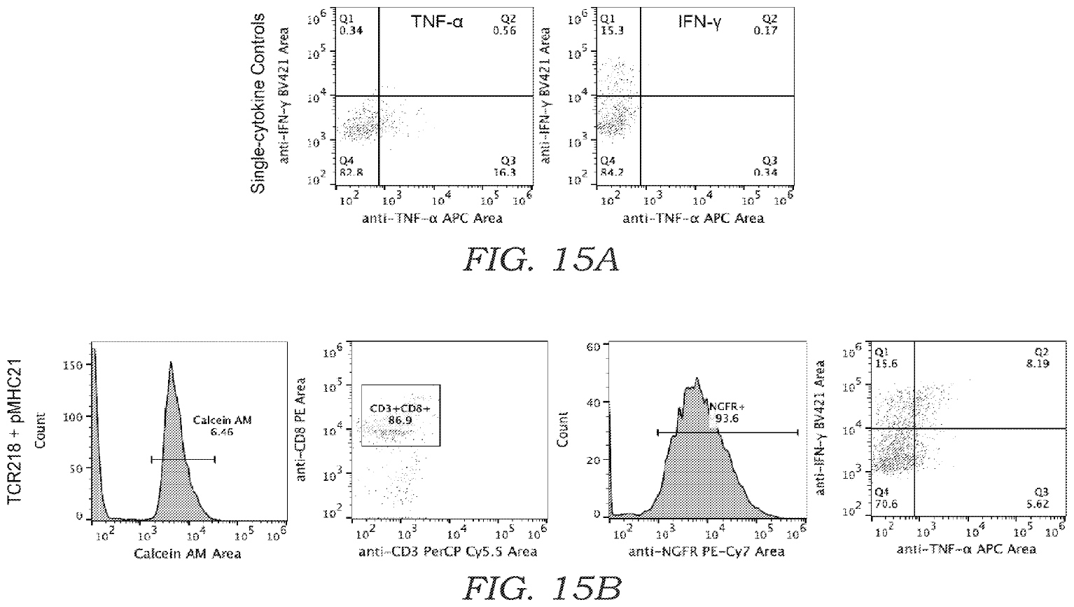

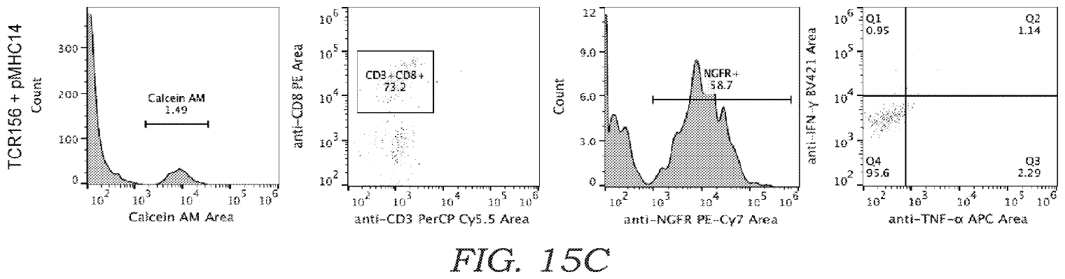

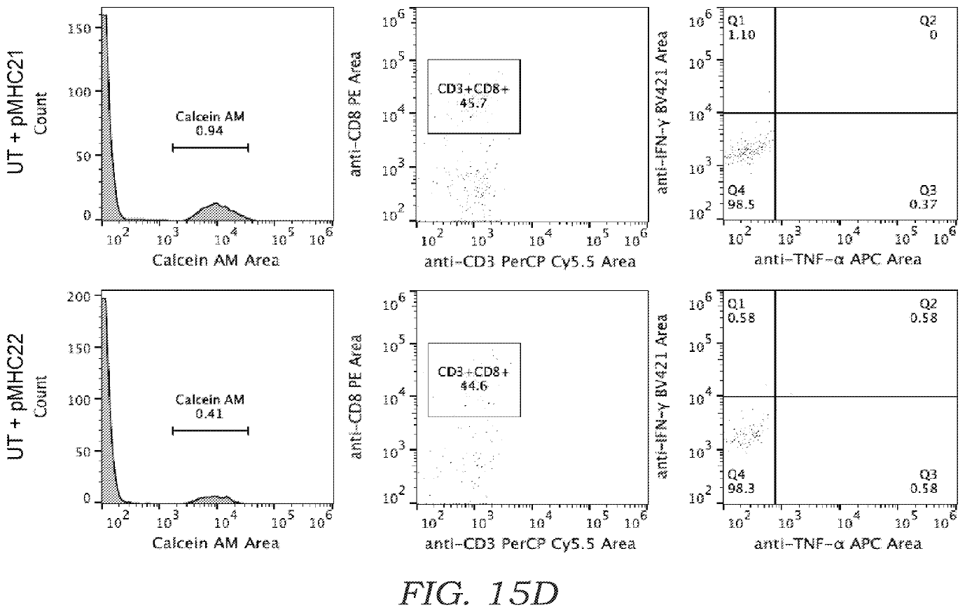

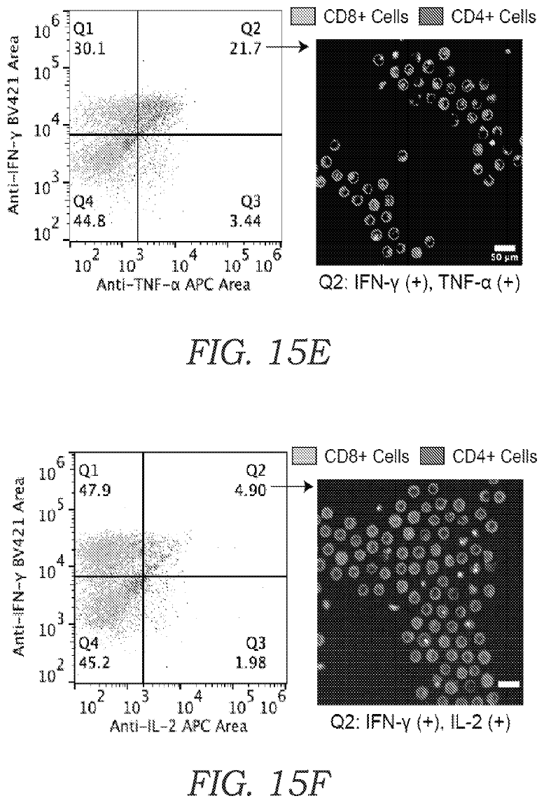

T cells engaged with APCs produce multiple cytokines simultaneously to achieve effector functions. The capability of nanovials 10 was further explored with additional anti-cytokine capture antibodies 18 to profile multiple cytokine secretions 22 and link this secretion phenotype with surface markers. First, it was tested whether the multiplexed secretion assay can be applied to low-potency TCRs targeting PAP-specific antigens. In the context of HLA-A*02:01, TCR128 and 218 transduced PBMCs were loaded onto nanovials 10 conjugated with PAP21 pMHC molecules 16. TCR156 transduced PBMCs were loaded onto PAP22 pMHC labeled nanovials 10, and the non-cognate PAP 14 pMHC-nanovials 10 acted as a negative control. Engineered CD3 CD8 cells 100 were highly enriched (NGFR⁺ %>90%) for all three tested PAP TCRs when loaded onto nanovials 10 with their cognate pMHC 16 (FIG. 6A. FIGS. 15A-15B), but not when using the non-specific PAP14 pMHC or when loading untransduced cells (FIGS. 15C-15D). CD3⁺CD8⁺ cells 100 representing a variety of secretion phenotypes, including an IFN-γ and TNF-α polyfunctional population, were successfully analyzed and sorted (FIG. 6B). The efficiency of nanovials 10 in detecting multiple cytokines 22 is similar for both strong tetramer signal (TCR128 and TCR218) and weak tetramer signal TCRs (TCR156).

Multiplexed-secretion profiling was further tested using untransduced human primary T cells activated with PMA and ionomycin coupled with CD8 and CD4 surface markers (FIG. 6C). Populations of cells 100 were sorted based on fluorescence peak areas exceeding the positive threshold for each individual cytokine 22 as well as combinations of IFN-γ and TNF-α or IL-2 and IFN-γ (FIGS. 15E-15F). When measuring IFN-γ and TNF-α, the dominant secretion phenotype for CD8 cells was IFN-γ (33.5%) and only a small fraction of CD8+ cells secreted TNF-α alone (4.29%) (FIG. 6D). About 24% of CD8+ cells were polyfunctional, secreting both IFN-γ and TNF-α simultaneously. On the other hand. CD4+ cells had a larger polyfunctional population (47.5%) and this pattern was consistent when analyzed for IFN-γ and IL-2 secretion (FIGS. 6D and 6E). The multiplexed secretion profiling capability of nanovials 10 could further improve the true discovery rate of novel TCRs based on unique secretion phenotypes, as well as provide links to gene expression responsible for such polyfunctionality of each secreting cell 100.

### DISCUSSION

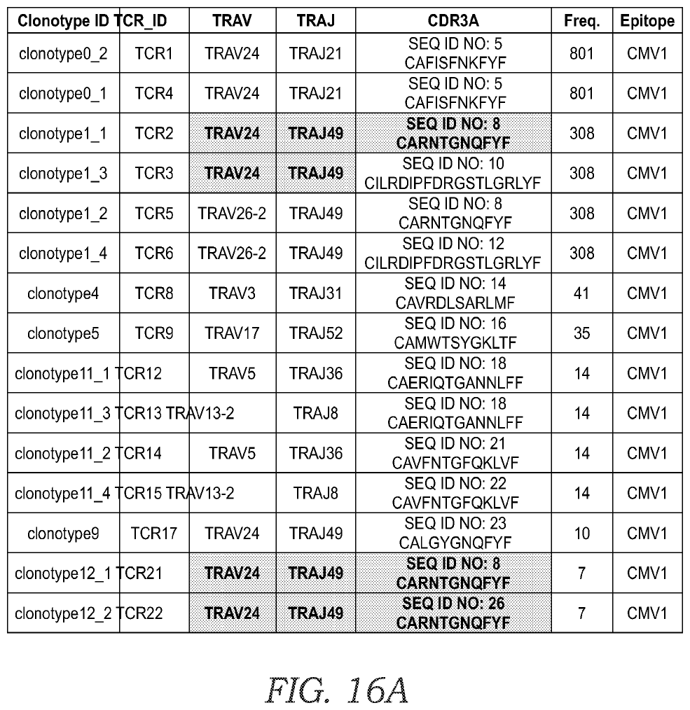

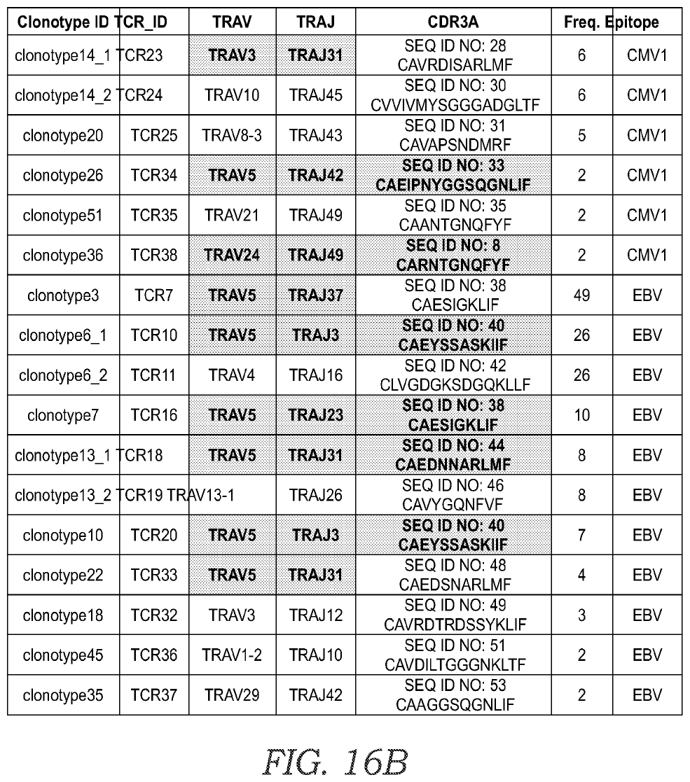

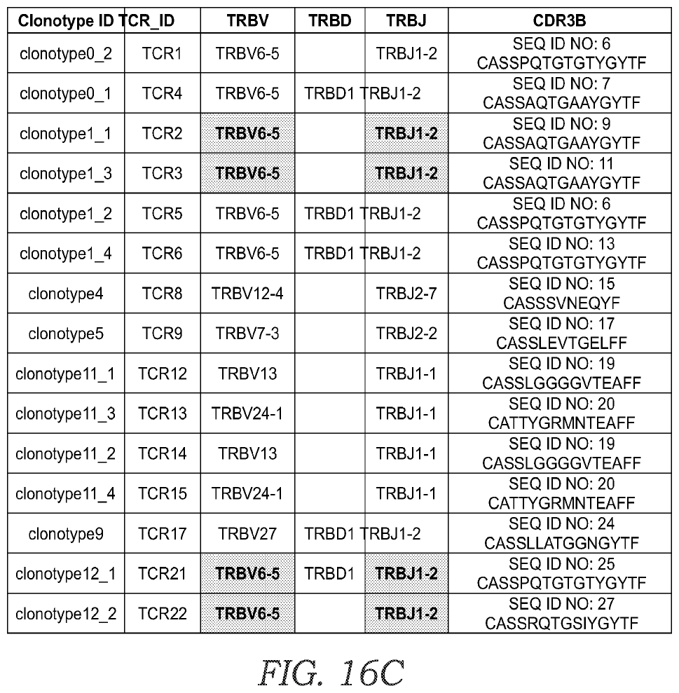

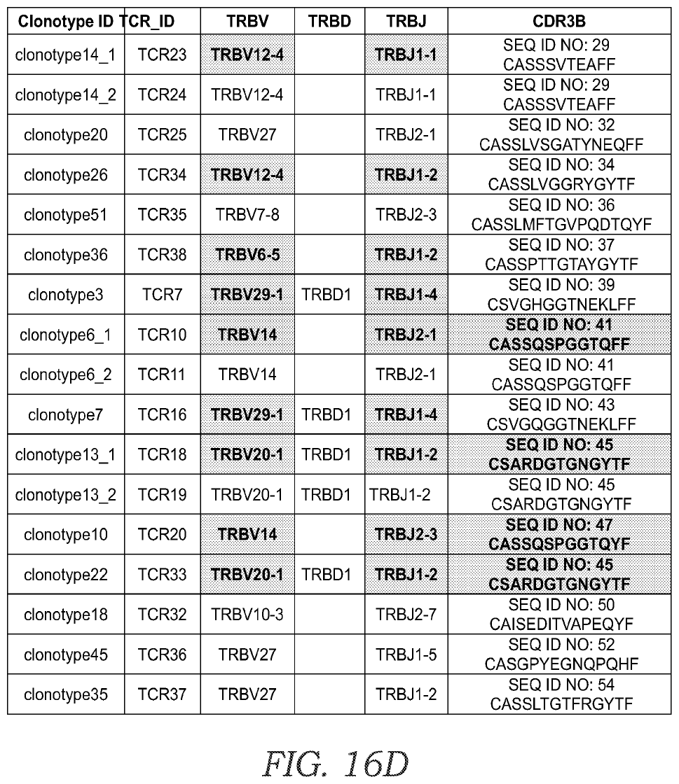

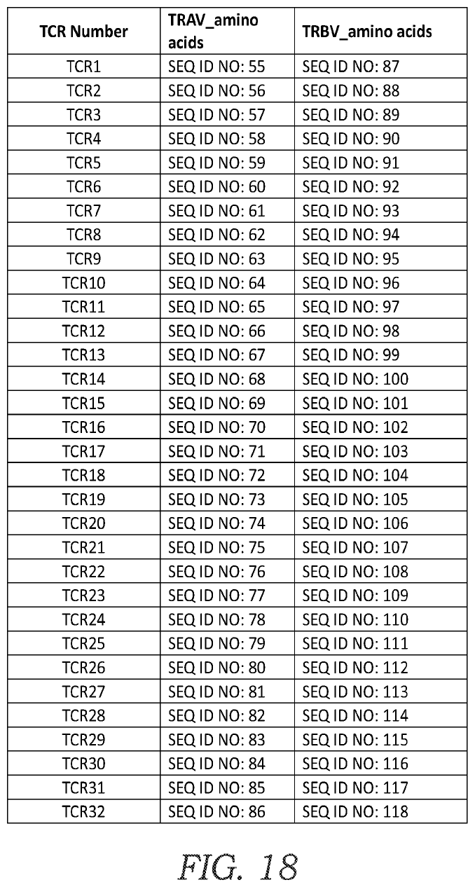

Nanovials 10 provide a tool to sort live antigen-specific T cells 100 based on a combination of TCR binding and functional response (cytokine or granzyme B secretion 22) followed by recovery of reactive TCRs and epitope-specific annotation. This approach brings a number of advantages over conventional single-cell cognate T cell isolation platforms. First, nanovials 10 can present pMHC molecules 16 at high density, providing an initial high avidity enrichment step from a large pool of cells 100 (˜20 million cells in these experiments). Even cells 100 with low affinity TCRs (5G6 and 9D2), which are not easily detectable using tetramer and dextramer staining, were recovered with higher purity. The ability to enrich a larger population of antigen-specific T cells 100 than conventional duo-tetramers without a pre-expansion process not only reduces a week-long workflow into a single day, but potentially enables rarer population of cells 100 to be identified. Nanovials 10 were able to recover some previously reported CMV1- and EBV-specific TCR sequences (bolded in FIGS. 16A-16D and also see FIG. 18) along with a diverse set of new TCR sequences that were validated to be functional (unbolded in FIGS. 16A-16D and see FIG. 18). This broader range does not come with the trade-off of low purity. High purity screening is supported by the 78% functional hit rate of CMV and EBV-specific TCR clonotypes following re-expression where each target epitope was accurately identified. Other large-scaled pooled barcoded multimer approaches demonstrated a functional hit rate of ˜50% or only assessed functionality of a few TCRs recovered instead of the entire set with 80% accuracy for calling matching epitopes.

Using barcoded secondary antibodies 26 to label secreted cytokines 22 allowed encoding of this cellular function into the single-cell sequencing data set and ranking of TCR sequences based on the amount of cytokine 22 secreted. The ability to link TCR sequence information directly to secretion levels of secretions 22 (e.g., cytokines) also appears to improve the ability to detect rare TCRs with higher confidence. Three new functional TCRs (FIG. 19) that are prostate cancer specific, from a pool of 25 that were re-expressed. None of the highest frequency clonotypes were functional upon re-expression. Notably, the TCRs associated with the highest granzyme B secretion barcode signals (2 of 6, 33%) were functional. As a comparison, in a previous study (Y. Pan et al., IRIS: Discovery of cancer immunotherapy targets arising from pre-mRNA alternative splicing. Proc Natl Acad Sci USA. 120 (2023)) 389 TCRs were re-expressed from more than 14 distinct healthy donors to retrieve 8 functional TCRs (2.05%). The results suggest functional-based screening can dramatically improve the functional recovery rate for rare TCRs.

The accessibility and compatibility of nanovials 10 with standard FACS and single-cell sequencing instrumentation can accelerate the development of personalized TCR immunotherapies. Epitopes for each recovered TCR are annotated through barcoding, while still being able to recover TCRs over a range of reactivity. Although only ten (10) different nanovial types were used, the number of pMHCs 16 that can be multiplexed with nanovials 10 is extensible to >40 based on commercial oligonucleotide-barcoding reagents, or ˜1000 using specialized manufacturing approaches. Since the TCR-pMHC interaction is heavily dependent on HLA-subtype restriction, the ability of nanovials 10 to provide TCRs along with matching HLA-restricted epitopes leverages current technology limitations to simultaneously profile a large library of antigen-specific T cells 100, especially in disease models identified with diverse HLA genotypes like type 1 diabetes or COVID-19.

By screening for TCRs based on the ability of T cells 100 to secrete a panel of cytokines 22, the links between TCR structure and cellular function can be further explored and discover therapeutically important TCRs that, for example, are used by different cell subsets, such as regulatory T cells to prevent autoimmune conditions. Recent work has emphasized the importance of functional characterization of TCRs, such as through assaying Ca²⁺ flux upon mechanical engagement of TCRs with pMHC-coated hydrogel beads, a platform that could be synergistic with nanovials 10 to more fully functionally screen TCRs. These types of multiomic studies can ultimately uncover relationships between TCR structure and function for improved efficacy in T cell therapies. Beyond TCRs, the nanovial assay format should be applicable to other screening processes, e.g., for CAR-T cells, CAR-NK cells, TCR-mimics, or bispecific T cell engagers (BiTEs), with minor adjustments, opening up a new frontier in functional screening for cell therapy discovery and development.

### Materials and Methods

### Nanovial Fabrication

Polyethylene glycol biotinylated nanovials 10 with 35 μm diameters were fabricated using a three-inlet flow-focusing microfluidic droplet generator (FIG. 7A), sterilized and stored at 4° C. in Washing Buffer consisting of Dulbecco's Phosphate Buffered Saline (Thermo Fisher) with 0.05% Pluronic F-127 (Sigma), 1% 1× antibiotic-antimycotic (Thermo Fisher), and 0.5% bovine serum albumin (Sigma) as previously reported. Additional details regarding fabrication of the nanovials 10 may be found in de Rutte J. et al., Suspendable Hydrogel Nanovials for Massively Parallel Single-Cell Functional Analysis and Sorting. ACS Nano. 2022 May 24; 16 (5):7242-7257, which is incorporated by reference herein.

### Nanovial Functionalization

Streptavidin conjugation to the biotinylated cavity of nanovials. Sterile nanovials 10 were diluted in Washing Buffer five times the volume of the nanovials (i.e., 100 μL of nanovial volume was resuspended in 400 μL of Washing Buffer). A diluted nanovial suspension was incubated with equal volume of 200 μg/mL of streptavidin (Thermo Fisher) for 30 minutes at room temperature on a tube rotator. Excess streptavidin was washed out three times by pelleting nanovials 10 at 2000×g for 30 seconds on a Galaxy MiniStar centrifuge (VWR), removing supernatant and adding 1 mL of fresh Washing Buffer.

Anti-CD45 and cytokine capture antibody labeled nanovials. Streptavidin-coated nanovials 10 were reconstituted at a five-time dilution in Washing Buffer containing 140 nM (20 μg/mL) of each biotinylated antibody or cocktail of antibodies: anti-CD45 (Biolegend, 368534) and anti-IFN-γ (R&D Systems, BAF285), anti-TNF-α (R&D Systems, BAF210), anti-IL-2 (BD Sciences, 555040). Nanovials 10 were incubated with antibodies for 30 minutes at room temperature on a rotator and washed three times as described above. Nanovials 10 were resuspended at a five times dilution in Washing Buffer or culture medium prior to each experiment.

pMHC labeled nanovials. MHC monomers with peptides of interest (pMHCs 16) were synthesized and prepared according to a published protocol. Streptavidin-coated nanovials 10 were reconstituted at a five times dilution in Washing Buffer containing 20 μg/mL biotinylated pMHC and 140 nM of anti-IFN-γ antibody or 140 nM of anti-granzyme B antibody (R&D systems, BAF2906) unless stated otherwise. For oligonucleotide barcoded nanovials 10, 1 μL of 0.5 mg/mL totalseq-C streptavidin (Biolegend, 405271, 405273, 405275) per 6 μL nanovial volume was additionally added during the streptavidin conjugation step.

### Cell Culture

Human primary T cells. Human primary T cells 100 were cultured as previously reported in Doyeon Koo et al, Sorting single T cells based on secreted cytokines and surface markers using hydrogel nanovials, bioRxiv Apr. 30, 2022, which is incorporated herein by reference.

Human donor PBMCs. To prime naïve T cells with peptides of interest. PBMCs from commercial vendors (AllCells) were cultured and processed as previously described with chemically synthesized peptides (>80% purity, Elim Biopharm). See Z. Mao et al., Physical and in silico immunopeptidomic profiling of a cancer antigen prostatic acid phosphatase reveals targets enabling TCR isolation, Proc Natl Acad Sci USA. 119, e2203410119 (2022), which is incorporated by reference herein.

K562 and Jurkat-NFAT-ZsGreen. K562 (ATCC) and Jurkat-NFAT-ZsGreen (gift from D. Baltimore at Caltech) were cultured in RPMI 1640 (Thermo Fisher) with 10% FBS (Omega Scientific) and Glutamine (Fisher Scientific). 293T (ATCC) was cultured in DMEM (Thermo Fisher) with 10% FBS and Glutamine.

### Nanovial Secretion Assay General Procedure

Cell loading onto nanovials. Each well of a 24-well plate was filled with 1 ml of media and 30 μL of reconstituted functionalized nanovials 10 (6 μL of nanovial volume=187,000 total nanovials 10) was added in each well using a standard micropipette. Cells 100 were seeded in each well and extra culture medium was added to make a total volume of 1.5 mL. Each well was mixed by simply pipetting 5 times with a 1000 μL pipette set to 1000 μL. The well plate was transferred to an incubator to allow cell binding; the volume in each well was pipetted up and down again 5 times with a 200 μL pipette set to 200 μL at 30-minute intervals. After one hour, nanovials 10 were strained using a 20 μm cell strainer to remove any unbound cells and recovered (FIG. 2C). During this step, any unbound cells were washed through the strainer and only the nanovials 10 (with or without cells 100 loaded) were recovered into a 12-well plate with 2 mL of media by inverting the strainer and flushing with media.

Activation, secretion accumulation and secondary antibody staining on nanovials. After cell loading. T cells 100 on nanovials 10 in a 12-well plate were activated via 10 ng/ml PMA (Sigma) and 2.5 μM ionomycin (Sigma) or the pMHCs 16 on the nanovials 10 for three hours in the incubator. Each sample was recovered in a conical tube with 5 mL wash buffer and centrifuged for 5 minutes at 200×g. Supernatant was removed and nanovials 10 were reconstituted at a ten-fold dilution in Washing Buffer containing detection antibodies 24 to label secreted cytokines 22 and/or cell surface markers. Concentrations of fluorescent antibodies per 187,000 nanovials (˜6 μL nanovial volume) are listed in Table 3, unless otherwise stated. A typical experiment used 30 μL of nanovials 10, which were incubated with 5× the volumes of antibodies listed in Table 3 (i.e., 25 μL anti-IFN-γ BV421, 10 μL anti-CD3 PerCP Cy5.5 and 10 μL anti-CD8 PE) at the total reaction volume of 300 μL. Nanovials 10 were incubated with the detection antibody cocktail at 37° C. for 30 minutes, protected from light. After washing nanovials 10 with 5 mL of Washing Buffer, nanovials 10 were resuspended at a 50-fold dilution in Washing Buffer and transferred to a flow tube.

TABLE 3
Secondary antibody concentrations per 6 μL nanovial volume.

| | Anti-IFN-γ | Anti-TNF-α | Anti-IL-2 | Anti-NGFR | Anti-CD8 | Anti-CD3 | Anti-CD3 | Anti-granzyme |
|---|---|---|---|---|---|---|---|---|
| Calcein AM | BV421 | APC | APC | PE-Cy7 | PE | PerCP Cy5.5 | APC Cy7 | B APC |
| Thermo Fisher | Biolegend | Biolegend | BD Sciences | Biolegend | Invitrogen | Biolegend | Biolegend | Biolegend |
| C3099 | 502532 | 502912 | 554567 | 345110 | 1208842 | 300430 | 300426 | 372203 |
| 0.3 μM | 5 μL of | 5 μL of | 5 μL of | 2 μL of | 2 μL of | 2 μL of | 2 μL of | 5 μL of |
| | 100 ug/mL | 100 μg/mL | 200 μg/mL | 100 μg/mL | 125 μg/mL | 100 μg/mL | 200 μg/mL | 100 μg/mL |

Labeling of secretion (granzyme B) using oligonucleotide barcoded secondary antibody. Following addition of fluorescent detection antibody 24 (anti-granzyme B APC), nanovials 10 were washed with 5 mL of Washing Buffer and reconstituted at a ten-fold dilution containing 30 nM of TotalSeq™ C-0987 anti-APC antibody 26 (Biolegend, 408007). Nanovial suspension was incubated at 37° C. for 30 minutes. After washing nanovials 10 with 5 mL of Washing Buffer, nanovials 10 were resuspended at a 50-fold dilution in Washing Buffer and transferred to a flow tube for FACS analysis and sorting (FIG. 2F).

Flow cytometer analysis and sorting. All flow cytometry analysis and sorting were performed using the SONY SH800 cell sorter equipped with a 130-micron sorting chip (SONY Biotechnology). The cytometer was configured with violet (405 nm), blue (488 nm), green (561 nm) and red (640 nm) lasers with 450/50 nm, 525/50 nm, 600/60 nm, 665/30 nm, 720/60 nm and 785/60 nm filters. Standard gain settings for different sensors are indicated in Table 4 below and gains were adjusted depending on the fluorophores used. In each analysis, samples were compensated using negative (blank nanovials 10) and positive controls (1000 ng/ml recombinant cytokine captured nanovials 10 labeled with each fluorescent detection antibody 24 or cells stained with each surface marker). Nanovial samples were diluted to approximately 623 nanovial/μL in Washing Buffer for analysis and sorting. Drop delay was configured using standard calibration workflows and single-cell sorting mode was used for all sorting as was previously determined to achieve the highest purity and recovery. A sample pressure of 4 was targeted. The following order of gating strategy was used to identify T cells 100 on nanovials 10 with strong secretion signal: 1) nanovial population based on high forward scatter height and side scatter area, 2) calcein AM positive population, 3) cell surface marker positive population (CD3, CD8, CD4 or NGFR), 4) cytokine secretion signal positive population based on fluorescence peak area and height.

TABLE 4
Common gain settings used for analysis and sorting.

| Sensor | FSC | BSC | FL1 | FL2 | FL3 | FL4 | FL5 | FL6 |
|---|---|---|---|---|---|---|---|---|
| Gain | 1 | 26% | 28% | 22% | 28% | 30% | 32% | 32% |

Dynamic Range of Cytokine Detection on Nanovials with a Combination of Antibodies

Nanovials 10 were labeled with biotinylated secretion capture antibodies 18 or cell surface marker antibodies (140 nM anti-CD45 and 140 nM anti-IFN-γ or anti-TNF-α) using the modification steps mentioned above. Each sample of cytokine capture antibody-labeled nanovials 10 was incubated with 0, 10, 100, or 1000 ng/ml of recombinant human IFN-γ (R&D Systems, 285IF100) and TNF-α (R&D Systems, 210TA020) for 2 hours at 37° C. Excess proteins were removed by washing nanovials 10 three times with Washing Buffer. Nanovials 10 were pelleted at the last wash step and incubated with anti-IFN-γ BV421 and anti-TNF-α APC as described in secondary antibody staining procedure and Table 3. Following washing three times, nanovials 10 were reconstituted at a 50 times dilution in the Washing Buffer and transferred to a flow tube. Fluorescent signal on nanovials was analyzed using a cell sorter with sensors and gains mentioned in the flow cytometer analysis and sorting section.

Maximum Binding of pMHC on Nanovials

Streptavidin coated nanovials 10 were functionalized with biotinylated HLA-A*02:01 restricted NY-ESO-1 pMHC by incubating at various concentrations (0, 20, 40, 80, 90, 100 μg/mL) and washed three times as described in Nanovial Functionalization section. Nanovials 10 were reconstituted at a ten-fold dilution in Washing Buffer containing 2 μL of 100 μg/mL anti-HLA-A2 FITC antibody (Biolegend. 343304) and incubated for 30 minutes at 37° C. After washing three times with Washing Buffer, mean fluorescence intensity was measured by flow cytometry.

### Single-T Cell Loading and Statistics

Nanovials 10 labeled with anti-CD45 antibodies were prepared using the procedures described above. To test cell concentration dependent loading of nanovials 0.15×10⁶ (0.8 cells per nanovial), 0.3×10⁶ (1.6 cells per nanovial), and 0.47×10⁶ (2.4 cells per nanovial) of cell tracker deep red stained human primary T cells 100 were each seeded onto 187,000 nanovials 10 in a 24-well plate and recovered as described above. Loading efficiency was analyzed using a custom image analysis algorithm in MATLAB. The software measured the total number of nanovials 10 in each image frame, then the number of cells 100 in each nanovial 10 was manually counted to record the total number of nanovials 10 with 0, 1 or 2 or more cells (n>2000). For comparing loading with different cell binding motifs, nanovials 10 were labeled with 140 nM of each biotinylated antibody: anti-CD3 (Biolegend, 317320), anti-CD3 and anti-CD28 (Biolegend. 302904), or anti-CD45. Nanovials 10 were seeded with 0.3 million cells in each well. To determine the effect of increased anti-CD45 concentration on nanovials 10, nanovials 10 were labeled by incubating with 0, 70, 140, or 210 nM of anti-CD45 antibodies and seeded with 0.3 million cells in a 24-well plate. After cell binding and recovery of nanovials 10, the number of cells 100 in each nanovial 10 was analyzed using the same image analysis algorithms mentioned above (n>2000).

### Analysis and Sorting of Human Primary T Cells Based on Secretion Level

Nanovials 10 were sequentially coated with streptavidin as described above and incubated with a solution of biotinylated antibodies (140 nM anti-CD45 and anti-IFN-γ, anti-TNF-α or anti-IL-2). 0.3 million human primary T cells 100 were seeded on nanovials 10 as described above and recovered into a 12-well plate in 2 mL of T cell expansion medium with PMA and ionomycin, followed by 3 hours of activation. Secreted cytokines 22 (IFN-γ, TNF-α, IL-2) were labeled with fluorescent detection antibodies 24 at concentrations described in Table 3 and cells were stained with calcein AM viability dye. After resuspending nanovials at 50-fold dilution in Washing Buffer, a small fraction of sample was transferred to a 96-well plate to be imaged using a fluorescence microscope prior to sorting. Pre-sort images were analyzed by custom image analysis algorithms in MATLAB. Fluorescence intensity profiles were calculated along a line segment manually defined around the cavity 12 of nanovial 10. The intensity peak height and the area under the intensity profile were then evaluated to find the peak area over height aspect ratio. Samples were analyzed using a cell sorter based on a combination of fluorescence area and height signals. To sort live single cells 100 based on secretion signal, nanovials 10 with calcein AM staining were first gated and high, medium, or low secretors were sorted by thresholding the fluorescence area and height signals. Sorted samples were imaged with a fluorescence microscope to validate the enrichment of nanovials 10 based on the amount of secreted cytokine 22 captured on the nanovials 10.

Capture, Activation, and Expansion of Antigen-Specific T Cells on pMHC-Labeled Nanovials

To determine the effect of pMHC concentration on antigen-specific T cell capture efficiency, streptavidin-coated nanovials 10 were functionalized by incubation with different concentrations of biotinylated HLA-A*02:01 NY-ESO-1 pMHCs (10, 20, 40, 80 μg/mL) and seeded with 0.3 million 1G4 TCR transduced PBMCs or untransduced PBMCs. After straining and recovery, samples were stained with calcein AM and anti-NGFR PE Cy7 antibody as described above. The fractions of nanovials 10 with live cells 100 and NGFR positive cells 100 were measured by a cell sorter. To test if activation was specific to the presence of pMHCs on nanovials, 1G4 transduced PBMCs were loaded onto anti-IFN-γ antibody and pMHC or anti-CD45 labeled nanovials 10. Following 3 hours of activation, nanovial samples were stained with anti-IFN-γ BV421 and anti-NGFR PE Cy7 antibodies. The fraction of nanovials 10 with NGFR positive cells and secretion signal was identified using flow cytometry. Secretion signal from 1G4 PBMCs on pMHC labeled nanovials 10 was measured at 0, 3, 6, and 12 hour time points. For detachment and expansion of antigen-specific T cells post-sort, 1G4 PBMCs loaded onto pMHC nanovials 10 were sorted based on calcein AM and NGFR signal and reconstituted with 0.75 mL media and 0.25 mL of 10 mg/mL Collagenase Type II solution (STEMCELL Technologies), followed by a 2-hour incubation at 37° C. Samples were vortexed 3 times at 20 second intervals and strained through a 20 μm strainer to remove empty nanovials 10. Cells cultured for 5 days were stained with 0.3 μM calcein AM and 0.02 mg/mL of propidium iodide or fluorescent anti-NGFR antibody and imaged using fluorescence microscopy or analyzed using flow cytometry for NGFR expression.

### Recovery of NY-ESO-1 TCR-Transduced Cells of Various Affinities

PBMCs were transduced with five different TCRs (1G4, 3A1, 4D2, 5G6, 9D2). 1 million of each TCR transduced or untransduced cells 100 were seeded with HLA-A*02:01 NY-ESO-1 pMHC and anti-IFN-γ labeled nanovials 10 and activated for 3 hours. Following straining of any unbound cells 100, recovered samples were stained with a cocktail of detection antibodies (calcein AM, anti-CD3 PerCP Cy5.5, anti-CD8 PE, anti-NGFR PE Cy7, anti-IFN-γ BV421) at concentrations described in Table 3. In parallel. 0.2 million PBMCs transduced with each TCR were stained with dual-color commercial HLA-A*02:01 NY-ESO-1 tetramers (MBL International, TB-M105-1 and TB-M105-2), anti-CD3 PerCP Cy5.5, and anti-CD8 PE antibodies. Using flow cytometric analysis, the purity of nanovial sample was calculated as the fraction of NGFR+ population from calcein AM+CD3+CD8+ cells on nanovials 10 or the fraction of NGFR+ population from calcein AM+CD3+CD8+ cells with IFN-γ secretion signal. The purity of the tetramer-stained samples was calculated as the fraction of NGFR+ population from CD3+CD8+ cells 100 with dual-color tetramer signal. For example, a detailed calculation for the purity of recovered 4A2 TCR transduced PBMCs is shown in Table 5.

TABLE 5
Calculation for the purity of recovered 4A2 TCR-specific T cells

| Tetramer | | Nanovials | |
|---|---|---|---|
| CD3+CD8+Tetramer+ | 38 | CD3+CD8+ Cells | 2537 |
| Cells | | CD3+CD8+IFN-γ+ Cells | 235 |
| CD3+CD8+Tetramer+NGFR+ | 15 | CD3+CD8+NGFR+ Cells | 1996 |
| Cells | | CD3+CD8+IFN-γ+NGFR+ | 217 |
| | | Cells | |
| Purity of | 40% | Purity of | 79% (due to |
| recovered sample | | recovered sample | binding alone) |
| | | | 92% (with |
| | | | secretion signal) |

### Isolation of Viral Epitope-Specific T Cells Using Nanovials, Tetramers and CD137 Staining

Nanovials 10 were functionalized with HLA-A*02:01 restricted pMHCs 16 targeting cytomegalovirus pp65, cytomegalovirus IE1 or Epstein-Barr virus BMLF1 with corresponding totalseq-C streptavidin barcodes C0971, C0972, C0973 (Biolegend, 405271, 405273, 405275) as described above. All sets of functionalized nanovials 10 were pooled together as one nanovial suspension (a total of 0.75 million nanovials). PBMCs were activated for 7 days with peptides associated with each antigen (CMV1: pp65/SEQ ID NO 2: (NLVPMVATV), CMV2: IE1/SEQ ID NO 3: (VLEETSVML), EBV: BMLF1/SEQ ID NO 4: (GLCTLVAML). 5 million activated PBMCs were loaded onto the pooled nanovial suspension. Following recovery and activation on nanovials ‘0 for 3 hours, samples were stained with viability dye and a cocktail of detection antibodies 24 (calcein AM, anti-CD3 APC Cy7, anti-CD8 PE, anti-IFN-γ). Using a cell sorter, viable CD3 and CD8 cells on nanovials 10 with IFN-γ secretion signal were sorted. In parallel, 5 million activated PBMCs were each stained with a surface activation marker (CD137) or CMV1 pMHC tetramers and sorted. All sorted samples were reconstituted in 18 μL of 1×PBS containing 0.04% BSA.

Direct Enrichment of Antigen-Specific T Cells without Pre-Activation Process

Nanovials 10 were functionalized with HLA-A*02:01 restricted CMV pp65 SEQ ID NO 2: (NLVPMVATV) pMHCs 16 and anti-IFN-γ antibody. 10⁷ freshly thawed PBMCs were directly loaded onto nanovials without 7 days of pre-activation with CMV pp65 peptide. Following recovery and activation on nanovials for 3 hours, samples were stained with detection antibody cocktail containing calcein AM, anti-CD3 APC Cy7, anti-CD8 PE and anti-IFN-γ at concentration described in Table 3. In parallel, 10⁷ of the same PBMCs were stained with anti-CD3 PerCp Cy5.5, anti-CD8 PE and CMV pp65 tetramer. Samples were analyzed using a cell sorter by gating to CD3+CD8+ cells on nanovials with IFN-γ signal or to CD3+CD8+ cells with tetramer signal.

Nanovial-Based Isolation of Prostate Cancer Epitope-Specific T Cells from One Donor

Nanovials 10 were functionalized with anti-granzyme B antibody and HLA-A*02:01 restricted pMHCs 16 each targeting ten (10) different prostate acid phosphatase (PAP) and cancer-enhanced splicing epitopes discovered in previous study (Mao et al., 2022, supra). Oligonucleotide streptavidin barcode 20 was also added to encode each pMHC molecule 16 on nanovials 10. PBMCs from one healthy donor were pre-activated for 7 days with peptides associated with each antigen: PAP14 SEQ ID NO 125: (ILLWQPIPV), PAP21 SEQ ID NO 126: (LLLARAASLSL), PAP22 SEQ ID NO 127: (TLMSAMTNL), PAP23 SEQ ID NO 128: (LLFFWLDRSVLA), CTNND1 SEQ ID NO 129: (MQDEGQESL), CLASP1 SEQ ID NO 130: (SLDGTTTKA), MEAF6 SEQ ID NO 131: (SGMFDYDFEYV), PXDN SEQ ID NO 132: (HLFDSVFRFL), SCAMP3 SEQ ID NO 133: (STMYYLWML), and TCF12 SEQ ID NO 134: (SLHSLKNRV). 20 million activated PBMCs were loaded onto the pooled nanovial suspension. Following recovery and activation on nanovials 10 for 3 hours, samples were stained with viability dye and a cocktail of detection antibodies 24 (calcein AM, anti-CD3 APC Cy7, anti-CD8 PE, anti-granzyme B APC). After washing, samples were also incubated with oligonucleotide anti-APC antibody 26. Using a cell sorter, viable CD3-CD8+ cells on nanovials 10 with granzyme B signal were sorted.

### Recovery of TCRs Using Single-Cell TCR αβ Sequencing

The standard protocol for 10× Chromium single cell 5′ and V(D)J enrichment with feature barcodes was followed unless otherwise noted. Sorted samples reconstituted at 18 μL were loaded into the 10× Chromium Next GEM Chip K for partitioning each nanovial 10 or T cell 100 into droplets 102 (FIG. 2G) containing primers specific for the constant region of the V(D)J locus allowing the PCR amplification and enrichment of matched a and B TCR sequences for individual cell barcoded cDNA. Single-cell TCR V(D)J and feature barcode libraries were constructed using the manufacturer-recommended protocol by the UCLA Technology Center for Genomics & Bioinformatics. Libraries were then sequenced on NextSeq 500 Mid Output with 2×150 bp (Illumina). The Cell Ranger VDJ pipeline was used for sample de-multiplexing and barcode processing.

For recovery of prostate cancer epitope-specific T cells 100 specifically. 10× Chromium single cell 5′ GEX and V(D)J enrichment with feature barcode system was utilized. Single-cell TCR V(D)J, 1^{st} feature barcode 20 for specific pMHC molecule 16 (of 10 types) on nanovial 10, 2^{nd} feature barcode 26 for granzyme B secretion level (oligo-anti-APC expression), and gene expression libraries were constructed using the manufacturer-recommended protocol. Libraries were then sequenced on NextSeq500. The Cell Ranger V(D)J pipeline was used for sample de-multiplexing and barcode processing. Gene expression data set was also analyzed using Cell Ranger Multi v6.1.2 pipeline with Human (GRCh38) 2020-A and Human (GRCh38) v5.0.0 references. FIG. 2H illustrates single cell TCR Recovery.

### Functional Validation of Recovered TCR Sequences

To measure antigen-specific reactivity of recovered CMV-, EBV- or cancer epitope-specific TCR sequences, TCRs were expressed and screened in Jurkat-NFAT-GFP cells as described in P. A. Nesterenko et al., HLA-A*02:01 restricted T cell receptors against the highly conserved SARS-CoV-2 polymerase cross-react with human coronaviruses. Cell Rep. 37 (2021), incorporated by reference. Paired TCR alpha and beta chains of interest were cloned into a retroviral pMSGV construct as previously described in M. T. Bethune et al., Isolation and characterization of NY-ESO-1-specific T cell receptors restricted on various MHC molecules. Proc Natl Acad Sci USA. 115, E10702-E10711 (2018), incorporated by reference herein. PBMCs for retroviral transduction were processed and cultured. To assess function of the transduced TCRs in human PBMCs. TCR expressing cells 100 were mixed with K562-A2 cells at a ratio of 1:2 (Effector:Target) in the RPMI media and supplemented with 1 μg/ml of anti-CD28/CD49d antibodies (BD Biosciences, 347690) and 1 μg/ml of cognate peptides or mixed peptide library. For PBMCs, supernatants were collected after 48 hours and analyzed by ELISA (BD Biosciences) to estimate IFN-γ concentration. PBMCs transduced by the vector without a TCR was used as a negative control.

### Multiplexed Secretion-Based Profiling to Identify Polyfunctional T Cells

Linking cell surface markers to secretion phenotype. Streptavidin-coated nanovials 10 were decorated with biotinylated secretion capture antibodies 18 (140 nM of anti-CD45, anti-IFN-γ and anti-TNF-α or anti-CD45, anti-IFN-γ and 140 nM anti-IL-2). Negative control nanovials 10 were prepared by labeling nanovials 10 only with anti-CD45 antibody without any cytokine capture antibodies 18. 0.5 million human primary T cells 100 were loaded onto nanovials 10 and recovered in T cell expansion medium containing 10 ng/ml PMA and 500 ng/ml ionomycin. Following 3 hours of activation, secreted cytokines 22 were stained with fluorescent detection antibodies 24 (anti-IFN-γ BV421, anti-TNF-α APC, anti-IL-2 APC) and cells were stained with 0.3 μM calcein AM, 5 μL of 25 μg/mL anti-CD4 PE (Biolegend, 344606) and 5 μL of 100 μg/mL anti-CD8 Alexa Fluor 488 (Biolegend. 344716) per 6 μL nanovial volume. Using a cell sorter. CD4 or CD8 cells on nanovials 10 with secretion signal were evaluated by first creating quadrant gates based on the negative control sample (nanovials 10 only labeled with anti-CD45 antibody). Q1 was defined as nanovials 10 with only IFN-γ secreting cells 100. Q2 was nanovials 10 with polyfunctional T cells 100 that secreted both cytokines 22 (IFN-γ and TNF-α or IL-2). Q3 was nanovials 10 with either TNF-α or IL-2 secreting cells 100 while Q4 was nanovials 10 with non-secretors. Nanovials 10 in each quadrant were sorted and imaged with a fluorescence microscope to quantify enrichment of each cell type and their associated secretion characteristics.

Multiplexed secretion-based profiling of cancer-specific cognate T cells. PBMCs were transduced with prostate acid phosphatase specific TCRs (TCR128, 156, 218) as previously described in Mao et al., supra. Streptavidin-coated nanovials 10 were functionalized with biotinylated anti-IFN-γ, anti-TNF-α secretion capture antibodies 18 and pMHC 16 targeting each TCR: PAP21 for both TCR128 and TCR218, and PAP22.

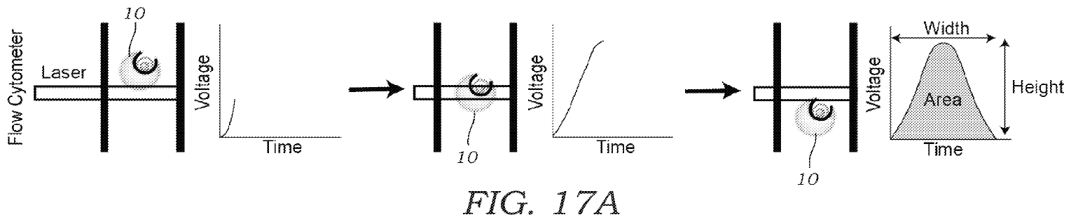

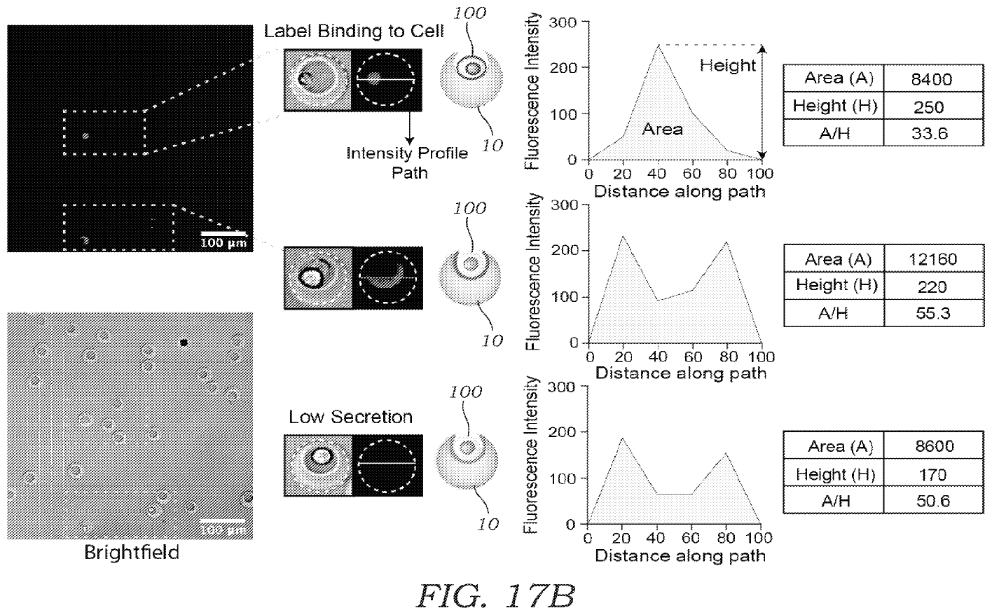

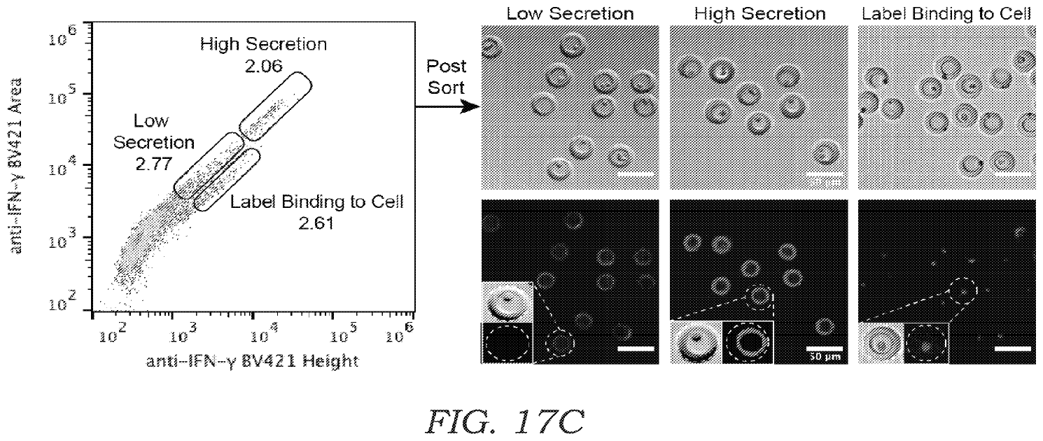

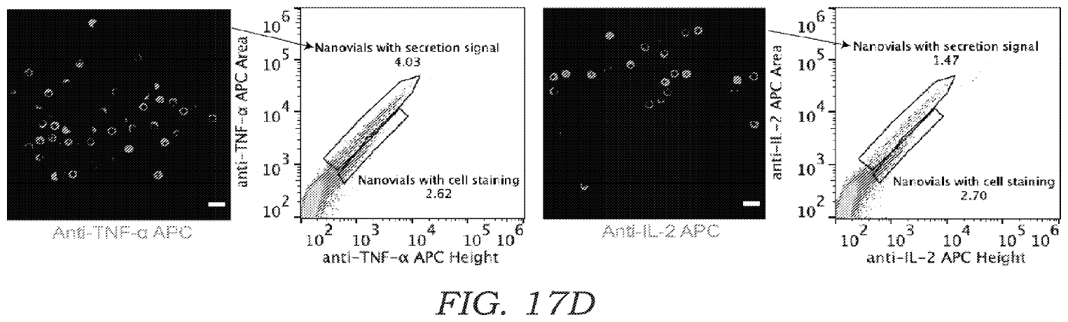

From fluorescence microscopy images, two distinct fluorescence patterns were observed; fluorescence spread across the nanovial cavity 12, presumably from secreted cytokines 22 and fluorescence associated with cells 100 on nanovials 10 (without signal on the nanovial 10). An approach was developed to use the fluorescence peak shape to distinguish between nanovial 10 and non-specific cell staining. From fluorescence images of T cells secreting on nanovials 10, the fluorescence intensity profile was plotted across the cavity 12 diameter using MATLAB and calculated the maximum intensity (height), the area under the intensity curve (area), and the ratio between the area and height (FIG. 17B). Nanovials 10 with both spatially spread secretion signal on their cavities and localized labels bound to the surfaces of adhered cells 100 were found to have a similar range of fluorescence peak height values. However, the area over height measurement was distinctly higher for the nanovials 10 with secretion signal. This information may be used as a distinguishing feature in flow cytometry, as analogous fluorescent pulses are generated when nanovials 10 pass through the excitation laser beam spot (FIG. 17A). The height of the flow cytometry pulse is determined by the maximum fluorescence intensity of the nanovial 10 and the area integrates the intensity emitted over the entire transit event through the laser spot. Accordingly, the nanovials 10 with spatially-distributed secretion signals are expected to produce higher fluorescence area signals for a given fluorescence intensity (height) compared to nanovials 10 with cells bound to labels. The samples were analyzed based on a combination of fluorescence peak area and peak height signals (area vs. height plot) and observed two populations, where one population had higher area signal as compared to the other population with similar height values. When nanovials 10 were sorted with larger ratios of area/height (2.06% high secretion and 2.77% low secretion gates), the recovered nanovials had higher secretion signals, while sorted events in the lower area/height (A/H) region (2.61% label binding to cell gate) corresponded to nanovials 10 with label bound to cells 100 (FIG. 17C). Using this area vs. height metric, one is able to sort populations of cells 100 with secreted cytokine signal only (A/H >3), completely differentiating secretion signal on 10 nanovials from signal solely from cell surface binding or intracellular staining of permeabilized or dead cells (FIG. 17D). The percent of the cell population with label bound was consistent across all three cytokines 22 (˜2.6% of the total analyzed events).

While embodiments of the present invention have been shown and described, various modifications may be made without departing from the scope of the present invention. For example, while cytokines have been a particular focus of the platform herein it should be appreciated that the systems and methods apply to other cell secretions. In addition, it should be understood that the oligonucleotide-labeled detection antibodies 22 may be specific for the actual cell secretions 22 or, alternatively, a fluorescently-labeled detection antibody 24. The invention, therefore, should not be limited, except to the following claims, and their equivalents.

What is claimed is:

1. A composition of matter comprising a polynucleotide encoding a T cell receptor (TCR) alpha chain polypeptide and/or a TCR beta chain polypeptide; wherein the polynucleotide is disposed in a vector, and when the vector is transduced into a CD8+ T cell, the TCR alpha chain polypeptide and/or the TCR beta chain polypeptide encoded by the polynucleotide form a T cell receptor on the CD8+ T cell that recognizes a polypeptide epitope present in a human MYST/Esa1 associated factor 6 (MEAF6) splicing variant polypeptide.

2. The composition of claim 1, wherein:
the T cell receptor recognizes a polypeptide epitope present in a human MYST/Esa1 associated factor 6 (MEAF6) splicing variant polypeptide in combination with a human leukocyte antigen HLA-A;
the T cell receptor recognizes a polypeptide epitope present in: SGMFDYDFEYV (SEQ ID NO: 131); or GMFDYDFEYV (SEQ ID NO: 135);
the polynucleotide encodes a segment of at least 10 amino acids having an at least 98% sequence identity to NVTCR21 (SEQ ID NO: 121 and/or SEQ ID NO: 124);
the polynucleotide encodes amino acids of a TCR variable region and the vector comprises vector polynucleotides encoding a TCR constant region fused in frame with the TCR variable region; and/or
the polynucleotide is disposed in a cell.

3. The composition of claim 2, wherein the cell is a human CD8+ T cell.

4. The composition of claim 3, wherein the cell is a CD8+ T cell obtained from an individual diagnosed with a cancer that expresses a human MEAF6 splicing variant; and the CD8+ T cell is transduced with a vector comprising a polynucleotide encoding a TCR Vα polypeptide in combination with a polynucleotide encoding a TCR Vβ polypeptide such that a heterologous TCR is expressed on a surface of the CD8+ T cell, wherein the heterologous TCR recognizes a MEAF6 splicing variant peptide associated with a human leukocyte antigen expressed on the surface of cells of the cancer.

5. A method of inhibiting growth of a prostate cancer cell or lung cancer cell comprising:
combining the prostate cancer cell or the lung cancer cell with a CD8+ T cell transduced with a polynucleotide encoding a T cell receptor (TCR) alpha chain polypeptide and a TCR beta chain polypeptide; wherein when transduced into and expressed in the CD8+ T cell, the alpha chain polypeptide and the TCR beta chain polypeptide can form a T cell receptor that recognizes a polypeptide epitope present in a human MYST/Esa1 associated factor 6 (MEAF6) splicing variant expressed on the prostate cancer cell or the lung cancer cell, thereby inhibiting growth of the prostate cancer cell or the lung cancer cell.

6. A composition of matter comprising a polynucleotide encoding a T cell receptor (TCR) alpha chain polypeptide and/or a TCR beta chain polypeptide; wherein the polynucleotide is disposed in a vector, and when the vector is transduced into a CD8+ T cell, the TCR alpha chain polypeptide and/or the TCR beta chain polypeptide encoded by the polynucleotide form a T cell receptor on the CD8+ T cell that recognizes a polypeptide epitope present in a human secretory carrier-associated membrane protein 3 (SCAMP3) polypeptide.

7. The composition of claim 6, wherein:
the T cell receptor recognizes a polypeptide epitope present in STMYYLWML (SEQ ID NO 133);
the T cell receptor recognizes a polypeptide epitope of SCAMP3 in combination with a human leukocyte antigen HLA-A*02:01;
the polynucleotide encodes amino acids of a TCR variable region and the vector comprises vector polynucleotides encoding a TCR constant region fused in frame with the TCR variable region;
the polynucleotide encodes a segment of at least 10 amino acids of a TCR variable region having an at least 98% sequence identity to NVTCR11 (SEQ ID NO: 119 and/or SEQ ID NO: 122); or NVTCR19 (SEQ ID NO: 120 and/or SEQ ID NO: 123); and/or
the polynucleotide is disposed in a cell.

8. The composition of claim 7, wherein the cell is a human CD8+ T cell.

9. The composition of claim 8, wherein the cell is a CD8+ T cell obtained from an individual diagnosed with a cancer that expresses a human SCAMP3 polypeptide; and the CD8+ T cell is transduced with a vector comprising a polynucleotide encoding a TCR Vα polypeptide in combination with a polynucleotide encoding a TCR Vβ polypeptide such that a heterologous TCR is expressed on a surface of the CD8+ T cell, wherein the heterologous TCR recognizes a SCAMP3 polypeptide associated with a human leukocyte antigen expressed on the surface of cells of the cancer.

10. A method of inhibiting growth of a breast cancer cell, a glioma cell or a hepatocarcinoma cell comprising:
combining the breast cancer cell, the glioma cell or the hepatocarcinoma cell with a CD8+ T cell transduced with a polynucleotide encoding a T cell receptor (TCR) alpha chain polypeptide and a TCR beta chain polypeptide; wherein when transduced into and expressed in the CD8+ T cell, the alpha chain polypeptide and the TCR beta chain polypeptide can form a T cell receptor that recognizes a polypeptide epitope present in a SCAMP3 polypeptide expressed on the breast cancer cell, the glioma cell or the hepatocarcinoma cell, thereby inhibiting growth of the breast cancer cell, the glioma cell or the hepatocarcinoma cell.

11. A composition of matter comprising a polynucleotide encoding a T cell receptor (TCR) alpha chain polypeptide and/or a TCR beta chain polypeptide; wherein the polynucleotide is disposed in a vector, and when the vector is transduced into a CD8+ T cell, the TCR alpha chain polypeptide and/or the TCR beta chain polypeptide encoded by the polynucleotide form a T cell receptor on the CD8+ T cell that recognizes a polypeptide epitope present in a cytomegalovirus (CMV) polypeptide.

12. The composition of claim 11, wherein:
the T cell receptor recognizes a polypeptide epitope present in NLVPMVATV (SEQ ID NO 2) or VLEETSVML (SEQ ID NO 3);
the T cell receptor recognizes a polypeptide epitope of CMV in combination with a human leukocyte antigen HLA-A*02:01;
the polynucleotide encodes amino acids of a TCR variable region and the vector comprises vector polynucleotides encoding a TCR constant region fused in frame with the TCR variable region; and/or
the polynucleotide is disposed in a cell.

13. The composition of claim 12, wherein the polynucleotide encodes a segment of at least 10 amino acids amino acids of a TCR variable region having an at least 98% sequence identity to amino acids of TCR1 (SEQ ID NO: 55 and/or SEQ ID NO: 87), TCR2 (SEQ ID NO: 56 and/or SEQ ID NO: 88), TCR3 (SEQ ID NO: 57 and/or SEQ ID NO: 89), TCR4 (SEQ ID NO: 58 and/or SEQ ID NO: 90), TCR5 (SEQ ID NO: 59 and/or SEQ ID NO: 91), TCR6 (SEQ ID NO: 60 and/or SEQ ID NO: 92), TCR8 (SEQ ID NO: 62 and/or SEQ ID NO: 94), TCR9 (SEQ ID NO: 63 and/or SEQ ID NO: 95), TCR12 (SEQ ID NO: 66 and/or SEQ ID NO: 98), TCR13 (SEQ ID NO: 67 and/or SEQ ID NO: 99), TCR14 (SEQ ID NO: 68 and/or SEQ ID NO: 100), TCR15 (SEQ ID NO: 69 and/or SEQ ID NO: 101), TCR17 (SEQ ID NO: 71 and/or SEQ ID NO: 103), TCR21 (SEQ ID NO: 75 and/or SEQ ID NO: 107), TCR22 (SEQ ID NO: 76 and/or SEQ ID NO: 108), TCR23 (SEQ ID NO: 77 and/or SEQ ID NO: 109), TCR24 (SEQ ID NO: 78 and/or SEQ ID NO: 110), or TCR25 (SEQ ID NO: 79 and/or SEQ ID NO: 111).

14. The composition of claim 13, wherein the cell is a CD8+ T cell obtained from an individual having undergone a stem cell transplantation and diagnosed with a CMV infection; and the CD8+ T cell is transduced with a vector comprising a polynucleotide encoding a TCR Vα polypeptide in combination with a polynucleotide encoding a TCR Vβ polypeptide such that a heterologous TCR is expressed on a surface of the CD8+ T cell, wherein the heterologous TCR recognizes a CMV polypeptide associated with a human leukocyte antigen expressed on the surface of cells infected with CMV.

15. A method of inhibiting cytomegalovirus (CMV) growth comprising:
combining a human cell infected with CMV with a CD8+ T cell transduced with a polynucleotide encoding a T cell receptor (TCR) alpha chain polypeptide and a TCR beta chain polypeptide; wherein when transduced into and expressed in the CD8+ T cell, the alpha chain polypeptide and the TCR beta chain polypeptide can form a T cell receptor that recognizes a polypeptide epitope present in a cytomegalovirus, thereby inhibiting growth of the cytomegalovirus.

16. A composition of matter comprising a polynucleotide encoding a T cell receptor (TCR) alpha chain polypeptide and/or a TCR beta chain polypeptide; wherein the polynucleotide is disposed in a vector, and when the vector is transduced into a CD8+ T cell, the TCR alpha chain polypeptide and/or the TCR beta chain polypeptide encoded by the polynucleotide form a T cell receptor on the CD8+ T cell that recognizes a polypeptide epitope present in a Epstein Barr virus (EBV) polypeptide.

17. The composition of claim 16, wherein:
the T cell receptor recognizes a polypeptide epitope present in GLCTLVAML (SEQ ID NO 4);
the T cell receptor recognizes a polypeptide epitope of EBV in combination with a human leukocyte antigen HLA-A*02:01;
the polynucleotide encodes amino acids of a TCR variable region and the vector comprises vector polynucleotides encoding a TCR constant region fused in frame with the TCR variable region; and/or
the polynucleotide is disposed in a cell.

18. The composition of claim 17, wherein the polynucleotide encodes a segment of at least 10 amino acids amino acids of a TCR variable region having an at least 98% sequence identity to amino acids of TCR7 (SEQ ID NO: 61 and/or SEQ ID NO: 93), TCR10 (SEQ ID NO: 64 and/or SEQ ID NO: 96), TCR11 (SEQ ID NO: 65 and/or SEQ ID NO: 97), TCR16 (SEQ ID NO: 60 and/or SEQ ID NO: 70), TCR18 (SEQ ID NO: 60 and/or SEQ ID NO: 72), TCR19 (SEQ ID NO: 73 and/or SEQ ID NO: 105), TCR20 (SEQ ID NO: 74 and/or SEQ ID NO: 106), or TCR32 (SEQ ID NO: 86 and/or SEQ ID NO: 118).

19. The composition of claim 18, wherein the cell is a CD8+ T cell obtained from an individual diagnosed with a head carcinoma or a neck carcinoma; and the CD8+ T cell is transduced with a vector comprising a polynucleotide encoding a TCR Vα polypeptide in combination with a polynucleotide encoding a TCR Vβ polypeptide such that a heterologous TCR is expressed on a surface of the CD8+ T cell, wherein the heterologous TCR recognizes a EBV polypeptide associated with a human leukocyte antigen expressed on the surface of cells infected with EBV.

20. A method of inhibiting Epstein Barr virus (EBV) growth comprising:
combining a human cell infected with EBV with a CD8+ T cell transduced with a polynucleotide encoding a T cell receptor (TCR) alpha chain polypeptide and a TCR beta chain polypeptide; wherein when transduced into and expressed in the CD8+ T cell, the alpha chain polypeptide and the TCR beta chain polypeptide can form a T cell receptor that recognizes a polypeptide epitope present in a Epstein Barr virus, thereby inhibiting growth of the Epstein Barr virus.
